(19) United States
(12) Patent Application Publication
(10) Pub. No.: US 2003/0119800 A1
(43) Pub. Date: Jun. 26, 2003

(54) Bone anabolic compounds and methods of use

(76) Inventors: Stavros C. Manolagas, Little Rock, AR (US); John A. Katzenellenbogen, Urbana, IL (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/165,380

(22) Filed: Jun. 7, 2002

Related U.S. Application Data
(60) Provisional application No. 60/299,009, filed on Jun. 18, 2001.

(51) Int. Cl. A61K 31/56

(52) U.S. Cl. 514/178; 514/182

(57) ABSTRACT

A variety of bone anabolic compounds are useful for maintaining and/or increasing bone mass, density, and/or strength in mammals. Preferred compounds enhance bone anabolic activity while minimizing or eliminating undesirable feminizing or masculinizing effects.

### RELATED APPLICATION INFORMATION

This application claims priority to U.S. Provisional Application No. 60/299,009, filed Jun. 18, 2001, which is hereby incorporated by reference in its entirety.

### FEDERAL FUNDING

This invention was funded in part through a grant from the National Institutes of Health. Therefore, the federal government has certain rights in this invention.

### BACKGROUND OF THE INVENTION

1. Field of the Invention

This invention relates to compounds useful for treating mammals, and particularly to compounds useful for maintaining or increasing bone mass and/or density and/or strength in humans, while minimizing or eliminating the undesirable effects of currently available treatments.

2. Description of the Related Art

In addition to their sine qua non role in the biology of reproduction, estrogens and androgens exert important regulatory influences on several non-reproductive tissues, including bone. Indeed, estrogen deficiency at menopause is responsible for one of the most common metabolic bone diseases of the modem era—postmenopausal osteoporosis. Prevention of this disease is the best justified rationale (and the only approved FDA indication) for prolonging estrogen replacement therapy for several decades after menopause. Based on the efficacy of estrogen replacement therapy in the prevention of osteoporosis and the assumption that the effects of estrogens on reproductive and non-reproductive tissues result from similar mechanisms of receptor action, replacement therapy with estrogens has been given during the last 60 years to millions of post-menopausal women in order to prevent the adverse effects of estrogen deficiency in reproductive and non-reproductive tissues alike. Deficiency of androgens (and probably estrogens) in males due to castration or a decline of production with old age, is also a major factor for the development of osteoporosis in men.

Osteoporosis is manifested as a decrease in bone mass and quality that leads to bone fragility and fractures. Bone is a dynamic tissue consisting of living cells and a matrix of proteins and minerals. It undergoes continual regeneration through a remodeling process that is accomplished by two types of highly specialized cells: osteoclasts, which remove old bone, and osteoblasts, which form new bone. Remodeling takes place mainly on the internal surfaces of bone and is carried out by temporary anatomical structures termed basic multicellular units (BMU's). These BMU's comprise teams of osteoclasts in the front and osteoblasts in the rear. As the BMU's travel over the bone surface, osteoclasts form excavation pits which are subsequently filled with new bone made by the osteoblasts that follow. Osteoclasts die by apoptosis (programmed cell death) and are quickly removed by phagocytes. During the longer lifespan of the osteoblasts (about 3 months, as compared to about three weeks for osteoclasts), some osteoblasts convert to lining cells that cover quiescent bone surfaces and some are entombed within the mineralized bone matrix as osteocytes. However, most of the osteoblasts die by apoptosis.

Most metabolic disorders of the adult skeleton, including osteoporosis, are believed to result from an imbalance between the resorption of old bone by osteoclasts and its subsequent replacement by osteoblasts. Sex steroids (estrogens or androgens) decrease the number of remodeling cycles by attenuating the birth rate of osteoclasts and osteoblasts. Consequently, a decline of sex steroids leads to an increased rate of bone remodeling. Sex steroids also modulate the lifespan of osteoclasts and osteoblasts, but in opposite directions, by regulating the process of apoptosis. Estrogen deficiency hastens the apoptosis of osteoblastic-osteocytic cells and delays the apoptosis of osteoclasts. Shortening the lifespan of the bone-forming osteoblasts and prolonging the lifespan of bone-resorbing osteoclasts tilts the balance between bone formation and resorption in favor of resorption. Hence, sex steroid deficiency leads to loss of bone and the development of osteoporosis.

Loss of ovarian function at menopause is a major risk factor for the development of osteoporosis as well as loss of libido, vasomotor disturbances known as hot flushes, unfavorable changes in lipoproteins, declining cognitive functions, and perhaps coronary artery disease, stroke and neurodegenerative diseases, like Alzheimer's. Estrogens are widely used for the treatment of menopausal symptoms and disorders in females, such as for maintaining bone mineral density, moderating hot flashes, enhancing cognition and the feeling of wellbeing, improving cardiovascular health, lowering blood lipids, etc. However, the estrogens typically used in these treatments, such as estradiol (Estrace®) or conjugated equine estrogens (Premarin®), have undesired effects. For example, they tend to stimulate the uterus and the breast, and thereby place these two tissues at increased risk for the development of cancer, as well as stimulating the growth of any existing estrogen-responsive cancer cells.

All currently approved drugs for the prevention and/or treatment of osteoporosis in the United States—estrogens, raloxifene, bisphosphonates, and nasal calcitonin spray—are antiresorptive agents, which decrease the development of osteoclast progenitors and/or recruitment and function of osteoclasts and slow the rate of bone remodeling. There are presently no approved therapies which can replace lost bone, e.g., bone lost as a result of osteoporosis, by raising bone mass from the high fracture risk range into the normal range. Daily injections of parathyroid hormone (PTH) for a period of 1.5 to 2 years can replace lost bone, and represent the first candidate for an anabolic bone therapy, i.e. a therapy that can truly increase bone mass. This treatment is currently pending FDA approval. So-called anabolic steroids have been considered in the past for the treatment of osteoporosis, but because of side effects including masculinizing changes in females, this form of treatment has fallen out of favor. Hence, with the exception of PTH, treatments approved or under consideration for osteoporosis are not bone anabolic agents that are able to replace lost bone, e.g., bone lost as a result of osteoporosis. Nor are these compounds, including estrogens and raloxifene, approved for the treatment of other disorders believed to be related to the estrogen deficiency of the postmenopausal state, such as coronary artery disease, stroke and neurodegenerative diseases.

### SUMMARY OF THE INVENTION

The inventors have discovered compounds that are Activators of Non-Genotropic Estrogen-like Signaling (“ANGELS”). ANGELS compounds are small molecules that mimic the non-genotropic effects of estrogen and androgen but substantially lack their genotropic effects. For example, the inventors have discovered that ANGELS compounds stimulate the formation of bone but have little or no feminizing or masculinizing effects.

Preferred embodiments provide methods comprising administering an ANGELS compound to a subject by a dosage regimen that is effective to increase or maintain a bone property selected from the group consisting of bone mass, bone density and bone strength. Preferably, the ANGELS compound is non-phenolic. In preferred embodiments, the ANGELS compound is selected from the group consisting of estrenediol, androstenediol, estranediol, androstanediol, nor-estrenediol, homo-estrenediol, seco-estrenediol, nor-androstenediol, homo-androstenediol, seco-androstenediol, nor-estranediol, homo-estranediol, seco-estranediol, nor-androstanediol, homo-androstanediol, seco-androstanediol, and estratrienol.

In preferred embodiments, the ANGELS compound is an estrenediol or an androstenediol. Preferably, the estrenediol is a 5(10)-estrenediol. Preferably, the 5(10)-estrenediol is selected from the group consisting of 5(10)-estrene-3α,17α-diol, 5(10)-estrene-3α,17β-diol, 5(10)-estrene-3β,17α-diol, and 5(10)-estrene-3β,17β-diol. Preferably, the ANGELS compound is a 5(6)-estrenediol or a 5(6)-androstenediol. Preferably, the ANGELS compound is selected from the group consisting of 5(6)-estrene-3α,17α-diol, 5(6)-estrene-3α,17β-diol, 5(6)-estrene-3β,17α-diol, 5(6)-estrene-3β,17β-diol, 5(6)-androstene-3α,17α-diol, 5(6)-androstene-3α,17β-diol, 5(6)-androstene-3β,17α-diol, and 5(6)-androstene-3β,17β-diol. Preferably, the ANGELS compound is a 4-estrenediol or a 4-androstenediol. Preferably, the ANGELS compound is selected from the group consisting of 4-estrene-3α,17α-diol, 4-estrene-3α,17β-diol, 4-estrene-3β,17α-diol, 4-estrene-3β,17β-diol, 4-androstene-3α,17α-diol, 4-androstene-3α,17β-diol, 4-androstene-3β,17α-diol, and 4-androstene-3α,17β-diol.

In preferred embodiments, the ANGELS compound is an estranediol or an androstanediol. Preferably, the ANGELS compound is selected from the group consisting of estrane-3α,17α-diol, estrane-3α,17β-diol, estrane-3β,17α-diol, estrane-3β,17β-diol, androstane-3α,17α-diol, androstane-3α,17β-diol, androstane-3β,17α-diol, and androstane-3β,17β-diol. Preferably, the ANGELS compound is a 5α-estranediol or a 5α-androstanediol. Preferably, the ANGELS compound is selected from the group consisting of 5α-estrane-3α,17α-diol, 5α-estrane-3α,17β-diol, 5α-estrane-3β,17α-diol, 5α-estrane-3β,17β-diol, 5α-androstane-3α,17α-diol, 5α-androstane-3α,17β-diol, 5α-androstane-3β,17α-diol, and 5α-androstane-3β,17β-diol. Preferably, the ANGELS compound is a 5β-estranediol or a 5β-androstanediol. Preferably, the ANGELS compound is selected from the group consisting of 5β-estrane-3α,17α-diol, 5β-estrane-3α,17β-diol, 5β-estrane-3β,17α-diol, 5β-estrane-3β,17β-diol, 5β-androstane-3α,17α-diol, 5β-androstane-3α,17β-diol, 5β-androstane-3β,17α-diol, and 5β-androstane-3β,17β-diol.

In preferred embodiments, the ANGELS compound is selected from the group consisting of nor-estrenediol, homo-estrenediol, seco-estrenediol, nor-androstenediol, homo-androstenediol, seco-androstenediol, nor-estranediol, homo-estranediol, seco-estranediol, nor-androstanediol, homo-androstanediol, and seco-androstanediol. Preferably, the ANGELS compound is selected from the group consisting of nor-estrenediol, homo-estrenediol, and seco-estrenediol. Preferably, the ANGELS compound is selected from the group consisting of nor-estranediol, homo-estranediol, and seco-estranediol. Preferably, the ANGELS compound is selected from the group consisting of nor-androstenediol, homo-androstenediol, and seco-androstenediol. Preferably, the ANGELS compound is selected from the group consisting of nor-androstanediol, homo-androstanediol, and seco-androstanediol.

In preferred embodiments, the ANGELS compound is an estratrienol. Preferably, the estratrienol is selected from the group consisting of estratrien-2-ol, estratrien-3-ol, estratrien-4-ol, and estratrien-5-ol. Preferably, the estratrienol is selected from the group consisting of seco-estratrienol, nor-estratrienol, and homo-estratrienol. Preferably, the estratrienol is selected from the group consisting of

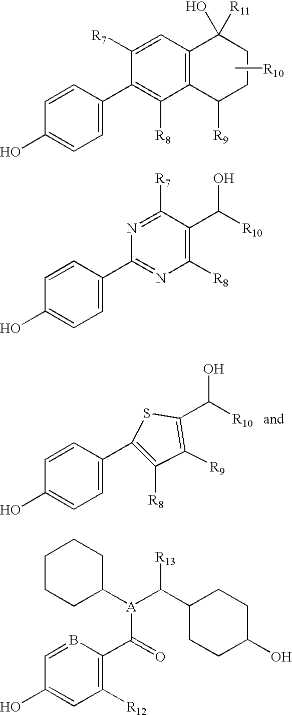

wherein R₇, R₈, R₉, R₁₀, R₁₁, and R₁₃ are each individually selected from the group consisting of hydrogen, C₁-C₅ alkyl and trifluoromethyl; A and B are each independently CH or N; and R₁₂ is selected from the group consisting of hydrogen, hydroxy, and C₁-C₅ alkyl. Preferably, R₇, R₈, R₉, R₁₀, R₁₁, and R₁₃ are each individually selected from the group consisting of hydrogen, methyl, ethyl, and trifluoromethyl.

In preferred embodiments, the ANGELS compound is selected from the group consisting of

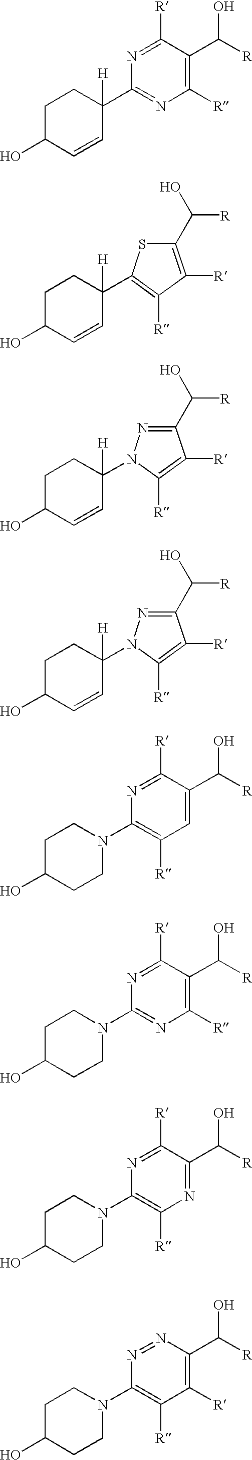

wherein R is hydrogen or C₁-C₅ alkyl; and wherein R′ and R″ are each individually selected from the group consisting of hydrogen, C₁-C₅ alkyl, trifluoromethyl, phenyl, and C₁-C₅ alkyl-substituted phenyl. Preferably, R is selected from the group consisting of hydrogen, methyl, and ethyl, and R′ and R″ are each individually selected from the group consisting of hydrogen, methyl, ethyl, propyl, trifluoromethyl, phenyl, 2-toluyl, 3-toluyl, and 4-toluyl.

In preferred embodiments, the ANGELS compound is selected from the group consisting of

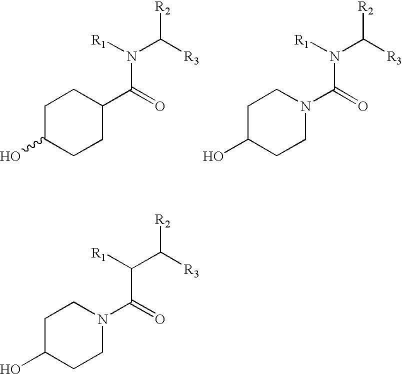

wherein R₁ is selected from the group consisting of hydrogen, C₁-C₅ alkyl, cycloalkyl, phenyl, and C₁-C₅ alkylphenyl; R₂ is selected from the group consisting of hydrogen, C₁-C₅ alkyl, and trifluoromethyl; and R₃ is selected from the group consisting of hydrogen, C₁-C₅ alkyl, cycloalkyl, hydroxycycloalkyl, phenyl, and C₁-C₅ alkylphenyl. Preferably, R₁ is selected from the group consisting of hydrogen, methyl, ethyl, isopropyl, cyclohcxyl, and phenyl; R₂ is selected from the group consisting of hydrogen, methyl, ethyl, isopropyl, and trifluoromethyl; and R₃ is selected from the group consisting of hydrogen, methyl, ethyl, isopropyl, phenyl, cyclohexyl, cyclopentyl, and 4-hydroxycyclohexyl.

In preferred embodiments, the subject to which the ANGELS compound is administered suffers from a bone disorder. Preferably, the bone disorder is selected from the group consisting of osteoporosis, Paget's disease, osteogenesis imperfecta, chronic hyperparathyroidism, hyperthyroidism, rheumatoid arthritis, Gorham-Stout disease, McCune-Albright syndrome, osteometastases of cancer, osteometastases of multiple myeloma and alveolar ridge bone loss. Preferably, the bone disorder is osteoporosis. Preferably, the osteoporosis is selected from the group consisting of postmenopausal, male, senile, glucocorticoid-induced, alcohol-induced, anorexia/amenorhea-related, immobilization-induced, weightlessness-induced, post-transplantation, migratory, idiopathic, and juvenile.

In preferred embodiments, the bone property increased or maintained by the administration of the ANGELS compound is bone mass, and/or bone density, and/or bone strength.

Additional preferred embodiments further provide methods comprising administering an ANGELS compound to a subject by a dosage regimen that is effective to provide a treatment selected from the group consisting of increase libido, control vasomotor disturbance, promote vasodilation, reduce bone loss, reduce mood swings, lower cholesterol, decrease low density lipoproteins (LDL), increase high density lipoproteins (HDL), slow atherosclerosis, slow progression of cancer, slow progression of cardiovascular disease, slow age-related neurodegeneration, slow progression of neurodegenerative disease, reduce risk of cancer, reduce risk of cardiovascular disease, reduce risk of stroke, and reduce risk of neurodegenerative disease. Preferably, the dosage regimen is effective to control a vasomotor disturbance or promote vasodilation. Preferably, the dosage regimen is effective to slow progression of cardiovascular disease, slow atherosclerosis, reduce risk of cardiovascular disease, or reduce risk of stroke. Preferably, the dosage regimen is effective to lower cholesterol, decrease LDL, or increase HDL. Preferably, the dosage regimen is effective to slow age-related neurodegeneration, slow progression of neurodegenerative disease, or reduce risk of neurodegenerative disease. Preferably, the dosage regimen is effective to increase libido. Preferably, the dosage regimen is effective to reduce bone loss. Preferably, the dosage regimen is effective to reduce mood swings. Preferably, the dosage regimen is effective to reduce risk of cancer or slow progression of cancer.

Additional preferred embodiments further provide ANGELS compounds, as well as pharmaceutical compositions comprising one or more of those compounds. A preferred embodiment provides a pharmaceutical composition comprising a compound represented by a formula selected from the group consisting of

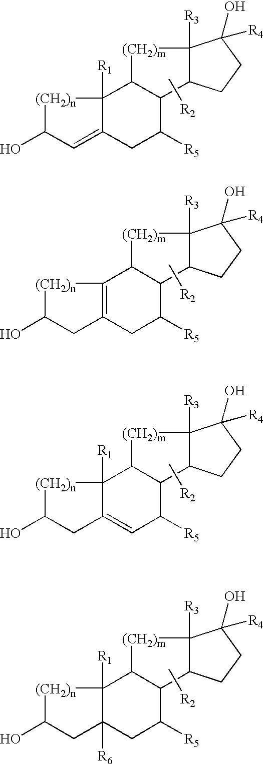

wherein R₁, R₃ and R₆ are each individually hydrogen or methyl; wherein m and n are each individually integers in the range of 1 to 3; and wherein R₂ and R₅ are each individually selected from the group consisting of hydrogen, halogen, mercapto, hydroxyl, cyano, amino, ethenyl, ethynyl, aryl, C₁-C₅ heteroaryl, C₁-C₅ alkyl, C₁-C₅ cycloalkyl, C₁-C₅ haloalkyl, C₁-C₅ alkylthio, C₁-C₅ ester, C₁-C₅ alkoxy, C₁-C₅ acyl, C₁-C₅ alkylamine, and C₁-C₅ acyloxy; and wherein R₄ is selected from the group consisting of hydrogen, ethenyl, ethynyl, aryl, C₁-C₅ heteroaryl, C₁-C₅ alkyl, C₁-C₅ cycloalkyl, C₁-C₅ haloalkyl, C₁-C₅ ester, and C₁-C₅ acyl.

In preferred embodiments, these compounds are represented by the following formula, in which the identities of m, n, and the various R groups are the same as given for the corresponding structure above:

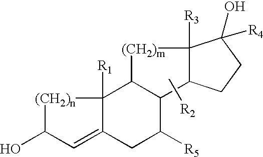

In preferred embodiments, n is 1 or 3 in the chemical structure shown immediately above. In preferred embodiments, m is 1 or 3 in the chemical structure shown immediately above. Preferably, these compounds are represented by the following formula, in which the identities of the various R groups are the same as in the corresponding generic structure provided above:

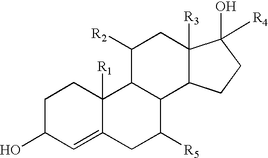

Preferably, in the structure shown immediately above, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

In other preferred embodiments, these compounds are represented by the following formula, in which the identities of m, n, and the various R groups are the same as given for the corresponding structure above:

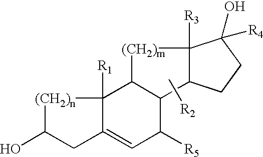

In preferred embodiments, n is 1 or 3 in the chemical structure shown immediately above. In preferred embodiments, m is 1 or 3 in the chemical structure shown immediately above. Preferably, these compounds are represented by the following formula, in which the identities of the various R groups are the same as in the corresponding generic structure provided above:

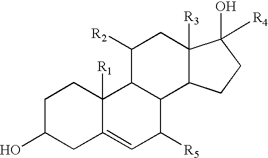

Preferably, in the structure shown immediately above, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

In other preferred embodiments, these compounds are represented by the following formula, in which the identities of m, n, and the various R groups are the same as given for the corresponding structure above:

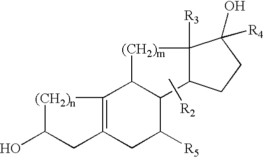

In preferred embodiments, n is 1 or 3 in the chemical structure shown immediately above. In preferred embodiments, m is 1 or 3 in the chemical structure shown immediately above. Preferably, these compounds are represented by the following formula, in which the identities of the various R groups are the same as in the corresponding generic structure provided above:

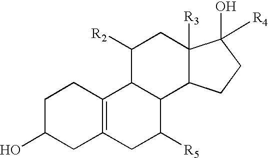

Preferably, in the structure shown immediately above, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

In other preferred embodiments, these compounds are represented by the following formula, in which the identities of m, n, and the various R groups are the same as given for the corresponding structure above:

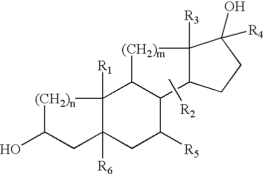

In preferred embodiments, n is 1 or 3 in the chemical structure shown immediately above. In preferred embodiments, m is 1 or 3 in the chemical structure shown immediately above. Preferably, these compounds are represented by the following formula, in which the identities of the various R groups are the same as in the corresponding generic structure provided above:

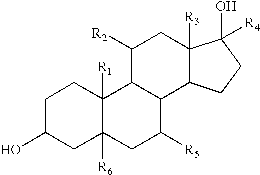

Preferably, in the structure shown immediately above, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

Additional preferred embodiments further provide ANGELS compounds, as well as pharmaceutical compositions comprising one or more of those compounds. A preferred embodiment provides a pharmaceutical composition comprising a compound represented by a formula selected from the group consisting of

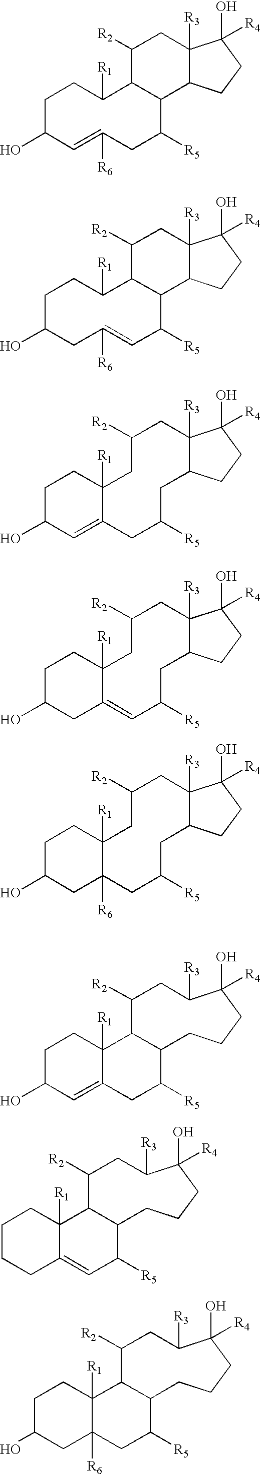

wherein R₁,R₃ and R₆ are each individually hydrogen or methyl; wherein R₂ and R₅ are each individually selected from the group consisting of hydrogen, halogen, mercapto, hydroxyl, cyano, amino, ehtenyl, aryl, C₁—C₅ heteroaryl, C₁-C₅ alkyl, C₁-C₅ cycloalkyl, C₁-C₅ haloalkyl, C₁-C₅ alkylthio, C₁-C₅ ester, C₁-C₅ alkoxy, C₁-C₅ acyl, C₁-C₅ alkylamine, and C₁-C₅ acyloxy; and wherein R₄ is selected from the group consisting of hydrogen, ethenyl, ethynyl, aryl, C₁-C₅ heteroaryl, C₁-C₅ alkyl, C₁-C₅ cycloalkyl, C₁-C₅ haloalkyl, C₁-C₅ ester, and C₁-C₅ acyl. In preferred embodiments, these compounds are represented by the following formulas, in which the identities of the various R groups are the same as given for the corresponding structures above:

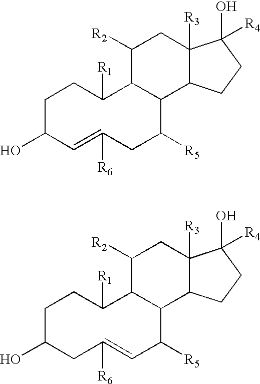

Preferably, in the structures shown immediately above, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

In other preferred embodiments, these compounds are represented by the following formulas, in which the identities of the various R groups are the same as given for the corresponding structures above:

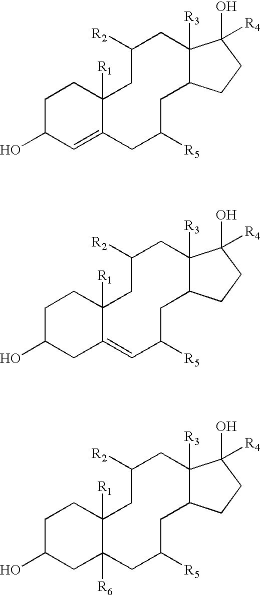

Preferably, in the structures shown immediately above, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

In other preferred embodiments, these compounds are represented by the following formulas, in which the identities of the various R groups are the same as given for the corresponding structures above:

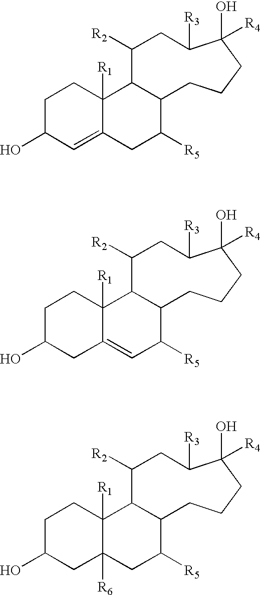

Preferably, in the structures shown immediately above, R₂ is selected from the group consisting of hydrogen, C₁-C₅ alkyl, phenyl, and C₁-C₅ alkyl-substituted phenyl; R₄ is selected from the group consisting of hydrogen, C₁-C₅ alkyl and ethynyl; and R₅ is selected from the group consisting of hydrogen and C₁-C₅ alkyl.

Additional preferred embodiments further provide ANGELS compounds, as well as pharmaceutical compositions comprising one or more of those compounds. A preferred embodiment provides a pharmaceutical composition comprising a compound represented by a formula selected from the group consisting of

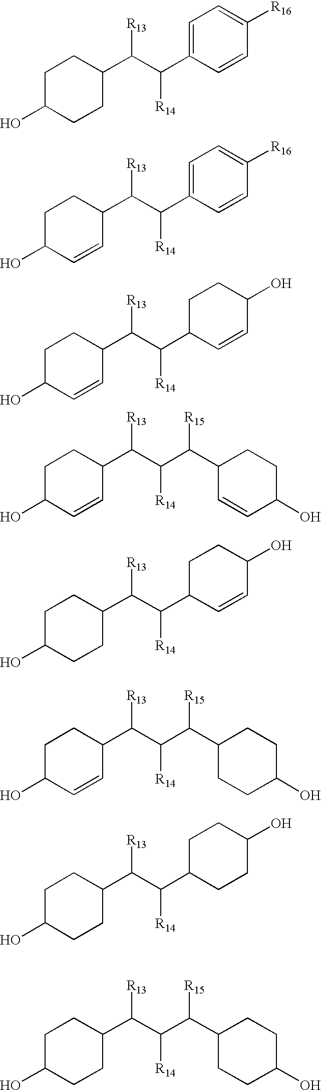

wherein R₁₃, R₁₄, and R₁₅ are each individually selected from the group consisting of hydrogen, ethenyl, ethynyl, C₁-C₅ alkyl, cycloalkyl and phenyl; and wherein R₁₆ is selected from the group consisting of hydrogen, hydroxyl, and C₁-C₅ hydroxyalkyl.

In other preferred embodiments, these compounds are represented by the following formulas, in which the identities of the various R groups are the same as given for the corresponding structures above:

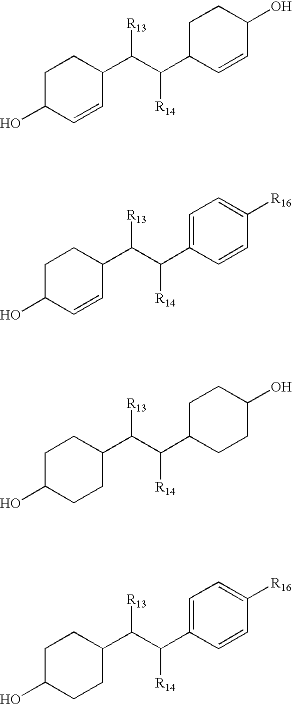

Preferably, in the structures shown immediately above, R₁₃ and R₁₄ are each individually selected from the group consisting of hydrogen, C₁-C₅ alkyl, cycloalkyl and phenyl; and R₁₆ is hydroxyl.

In other preferred embodiments, these compounds are represented by the following formulas, in which the identities of the various R groups are the same as given for the corresponding structures above:

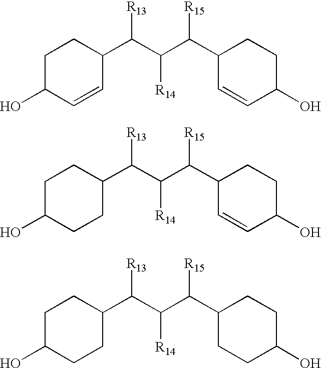

Preferably, in the structures shown immediately above, R₁₃, R₁₄ and R₁₅ are each individually selected from the group consisting of hydrogen, C₁-C₅ alkyl, cycloalkyl and phenyl.

In other preferred embodiments, these compounds are represented by the following formulas, in which the identities of the various R groups are the same as given for the corresponding structures above:

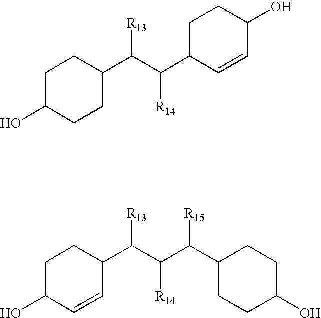

Preferably, in the structures shown immediately above, R₁₃, R₁₄ and R₁₅ are each individually selected from the group consisting of hydrogen, C₁-C₅ alkyl, cycloalkyl and phenyl.

Additional preferred embodiments further provide ANGELS compounds, as well as pharmaceutical compositions comprising one or more of those compounds. A preferred embodiment provides a pharmaceutical composition comprising a compound represented by a formula selected from the group consisting of

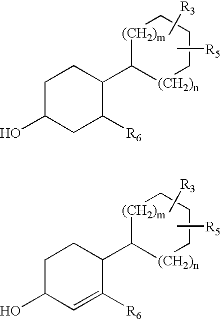

in which m and n are each individually integers in the range of 1 to 4; R₃ and R₅ are each individually selected from the group consisting of hydroxy, hydrogen, C₁ to C₅ alkyl, C₁ to C₅ hydroxyalkyl, C₁ to C₅ alkoxy, C₁ to C₅ thioalkoxy, phenyl, and C₁ to C₅ alkyl-substituted phenyl; and in which R₆ is selected from the group consisting of hydrogen and C₁-C₅ alkyl.

In other preferred embodiments, these compounds are represented by the following formulas, in which the identities of the various R groups are the same as given for the corresponding structures above:

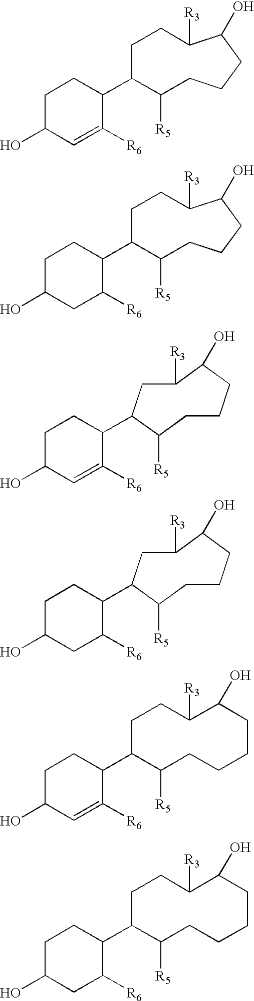

Preferably, in the structures shown immediately above, R₃ is selected from the group consisting of hydrogen, methyl and ethyl; and R₅ and R₆ are each individually selected from the group consisting of hydrogen and C₁-C₅ alkyl.

### BRIEF DESCRIPTION OF THE DRAWINGS

These and other aspects of the invention will be readily apparent from the following description and from the appended drawings, which are meant to illustrate and not to limit the invention, and wherein:

Scheme 1A illustrates the chemical structures of various preferred estrenes, estranes, androstenes and androstanes.

Scheme 1B illustrates the general structure of preferred estrenes, estranes, androstenes and androstanes.

Scheme 1C illustrates preferred syntheses of various estrenes, estranes, androstenes and androstanes.

Scheme 2A illustrates a general structure for estrene, estrene analogs, and derivatives with potency-modifying substituents.

Scheme 2B illustrates preferred syntheses of estrene analogs with potency-modifying substituents.

Scheme 3A illustrates the chemical structures of preferred homo-, nor-, seco- and cyclo-analogs of estrenes.

Scheme 3B illustrates the general structure of preferred homo-, nor-, seco- and cyclo-analogs of estrenes.

Scheme 3C illustrates preferred syntheses of various homo-, nor-, seco- and cyclo-analogs of estrenes.

Scheme 4A illustrates various preferred heterocyclic and heteroacyclic analogs of estrenes.

Scheme 4B illustrates the general structure of preferred heterocyclic and heteroacyclic analogs of estrenes.

Scheme 4C illustrates preferred syntheses of various heterocyclic analogs of estrenes.

Scheme 4D illustrates preferred syntheses of various heteroacyclic analogs of estrenes.

Scheme 5A illustrates the chemical structures of various preferred estratriene analogs.

Scheme 5B illustrates the general structure of preferred estratrienol analogs.

Scheme 5C illustrates preferred syntheses of various carbocyclic estratrienol analogs.

Scheme 5D illustrates preferred syntheses of various heterocyclic-core and heteroacyclic-core estratrienol analogs

FIGS. 1A-F illustrates that nongenotropic activation of cytoplasmic kinases and downstream transcription-dependent and -independent events are required for the anti-apoptotic effects of sex steroids.

FIG. 2 illustrates that the transcriptional regulation of SRE-SEAP by estrogens requires the Src/Shc/ERK signaling pathway.

FIG. 3 illustrates that the transcriptional regulation of AP-1-SEAP by estrogens requires the JNK signaling pathway.

FIG. 4 illustrates that SRE- and AP-1-dependent transcription is exerted via a sex-nonspecific, nongenotropic mechanisms.

FIGS. 5A-B illustrates that estradiol-induced phosphorylation of Elk-1 is required for ERa-mediated activation of SRE-SEAP.

FIG. 6 illustrates that transcriptional effects involving regulation of Elk-1, C/EBPβ, CREB and JNK1/AP-1 are required for the anti-apoptotic effect of sex steroids via either the ER or the AR.

FIGS. 7A-D illustrates equivalence of the skeletal, but not the reproductive, actions of estrogens and androgens in female and male mice.

FIG. 8 illustrates that the pro-apoptotic effect of sex steroids on osteoclasts requires Src/ERK signaling.

FIGS. 9A-D illustrates the equivalence of the skeletal actions of estrogens and androgens in female and male mice.

FIGS. 10A-C illustrates the relative binding affinity of 4-estren-3α,17β-diol (ABX102) to full length, human ERα and ERβ.

FIGS. 11A-C illustrates increased bone density in gonadectomized mice receiving 4-estren-3α,17β-diol (4-Ed).

FIGS. 12A-C illustrates increased vertebral compression strength, preservation of marrow cavity and prevention of osteoblast apoptosis in mice receiving 4-estren-3α,17β-diol (4-Ed).

FIGS. 13A-G illustrates increased trabecular and cortical width, osteoblast number and serum osteocalcin in ovariectomized mice receiving 4-estren-3α,17β-diol (4-Ed).

FIGS. 14A-D illustrates a lack of an effect of 4-estren-3α,17β-diol (4-Ed) on female and male reproductive tissues or breast cancer cells.

FIGS. 15A-B illustrates the results of a screen for genotropic vs. nongenotropic activity of compounds related to 4-estren-3α,17β-diol (4-ED) (from scheme 1A).

### DETAILED DESCRIPTION OF THE PREFERRED EMBODIMENTS

In preferred embodiments, this invention involves ANGELS compounds and methods of using these compounds to enhance health and well being. ANGELS compounds are small (molecular weight of about 1,000 or less) compounds that mimic the non-genotropic effects of estrogen and androgen but substantially lack their genotropic effects. Preferred ANGELS compounds are non-phenolic, and thus are not estrogens. In humans, the non-genotropic effects of estrogen and androgen include a number of bone anabolic, atheroprotective and neuroprotective functions. Examples of such non-genotropic effects may include promotion of vasodilation, suppression of hot flushes, reduction of bone loss, increase of bone density, increase of bone mass, increase of bone strength, reduction of mood swings, lowering of cholesterol, slowing of atherosclerosis, slowing the progression of cancer, slowing the progression of cardiovascular disease, slowing the progression of neurodegenerative disease, reducing the risk of cancer, reducing the risk of cardiovascular disease, reducing the risk of stroke, and/or reducing the risk of neurodegenerative disease.

However, the beneficial effects of maintaining or supplementing estrogen or androgen levels in humans are limited by their genotropic side effects. These genotropic effects are typically manifested as uterine, breast and/or ovarian cancers, and/or clinically significant feminizing or masculinizing effects when given to the opposite sex. For example, administration of estrogen to men by dosage regimens that are effective to produce beneficial non-genotropic effects also tends to produce undesirable feminizing effects such as breast growth, (gynecomastia), breast pain (mastodynia), and decreased hair growth, as well as decreased ejaculate volume and decreased sperm count. Likewise, administration of androgens to females by dosage regimens effective to produce beneficial non-genotropic effects also tends to produce undesirable masculinizing effects such as growth of facial hair, (hirsutism), acne, laryngeal enlargement, deepening voice, muscular hypertrophy, enlargement of clitoris (clitoromegaly), and amenorrhea.

Preferred ANGELS compounds at least partially restore osteoporotic bone to normal mass, density and/or and strength, which is not possible with currently approved therapies, and preferably also provide other beneficial effects of estrogens and/or androgens, with clinically insignificant cancer risk for reproductive organs, and without clinically significant masculinizing or feminizing side effects. ANGELS compounds are not SERMs, as that term is currently understood. SERMs are estrogen agonists in bone and the cardiovascular system, but antagonists in the uterus or the breast. For example, raloxifene is a weak estrogen agonist in bone, and only in the absence of estrogens. Both raloxifene and tamoxifen, another SERM, are antagonists on bone in the presence of estrogens, e.g., in pre-menopausal women. In other words, SERMs cause loss of bone in the estrogen sufficient state. SERMs can, at best, be as good as estrogens in bone, but estrogens are no longer considered to be the standard of care for treatment of osteoporosis. In addition, recent evidence indicates that SERMs are ineffective for men. Finally, raloxifene is an antagonist of estrogen on the vasomotor system, and exacerbates hot flushes.

As shown below, it is believed that ANGELS compounds work by an entirely different mechanism than either estrogens or SERMS. This invention is not limited by any theory of operation, but the data shows that sex steroids protect the adult skeleton through a fundamentally distinct mechanism of receptor action than that utilized to preserve the mass and function of reproductive organs or to stimulate the proliferation of breast cancer cells. Specifically, whereas the classical genotropic action of sex steroids receptors is essential for their effects on reproductive tissues, this action is dispensable for their bone protective effects. For example, it is believed that estrogens or androgens exert anti-apoptotic effects on osteoblasts and pro-apoptotic effects on osteoclasts through a non-genotropic regulation of MAP kinases and downstream transcription-dependent and -independent events. ANGELS compounds substantially reproduce these non-genotropic effects without affecting classical transcription. For example, it has now been discovered that whereas sex steroids prevent bone loss, preferred ANGELS compounds increase bone mass and/or density and/or strength in either sex without affecting reproductive organs.

Preferred ANGELS compounds are superior to estrogens on bone, while displaying little or no uterine or breast activity. In addition, preferred ANGELS compounds are effective in males because the feminizing effects are clinically insignificant. Also, preferred ANGELS compounds work like estrogens on the vasomotor system by decreasing hot flushes. Preferred ANGELS compounds are classified into four categories as described below. These categorizations are for the sake of convenience and are not to be regarded as limiting the scope of the invention. It is understood that the recitation of particular compounds and/or classes of compound herein includes stereoisomers, salts, derivatives and metabolites thereof. Thus, those skilled in the art will appreciate that the various structural formulas described herein represent all stereoisomers.

Category I: Estrenes, Estranes, Androstenes, and Androstanes

Examples of ANGELS compounds included in Category I are shown in Scheme 1A, and a general structure encompassing other analogs and derivatives is shown in Scheme 1B. A number of the simple members of Category I are known compounds, some of which are commercially available (for example, from Steraloids Inc., Newport, R.I.). Analogs in which the stereochemistry of various ring junction and fusion positions is inverted from that which is typical in the natural steroids (i.e., 5α, 8β, 9α, 10β, 13β, 14α, 17β) are included in Category I, preferably those with 5β and/or 17α configurations. All of these analogs may be prepared using well-established approaches to the total synthesis of steroids or by the conversion of steroids that are known and/or commercially available (Steraloids Inc., Newport, R.I.) into the novel analogs and derivatives. Standard methods for steroid synthesis and steroid conversion reactions may be found in the following references: Fieser and Fieser, 1967; Fried and Edwards, 1972; Kirk and Hartshorn, 1968; Shoppee, 1964; and Djerassi, 1963. Examples of syntheses of some members of Category I are shown in Scheme 1C.

The potency and efficacy of members of Category I can be enhanced by substitution at various positions, preferably the 7α, 11β, and 17α positions in the manner shown in Scheme 2A, providing increased potency for selective bone anabolic activity. Preferred substituents at all three positions include halogen, heteroatom, and substituted heteroatom groups, alkyl, alkenyl, alkynyl, aryl and heteroaryl groups, alkyl, alkenyl, alkynyl, aryl, heteroaryl, halogen and heteroatom-substituted analogs of the preceding substituents, and cyclic analogs of the alkyl and alkenyl substituents. More preferred substituents at all three positions include small halogen or substituted (C₁-C₄) heteroatoms, small alkyl or cycloalkyl groups (C₁-C₅), small alkenyl or alkynyl groups (C₂-C₆), small aryl and heteroaryl groups, and alkyl, alkenyl, alkynyl, aryl, heteroaryl, halogen and heteroatom-substituted analogs of the preceding substituents bearing small substituents (C₁-C₄). Highly preferred substituents include, at the 7α position, small halogen (F, Cl, or Br) or heteroatoms with small (C₁-C₂) alkyl substituents. At the 11β position, highly preferred substituents include small alkyl groups (C₁-C₃) with or without small halogens (F, Cl, Br), or with heteroatoms bearing small (C₁-C₂) alkyl substituents, alkenyl, alkynyl, aryl or heteroaryl groups without or with small alkyl (C₁-C₃) with or without small halogen (F, Cl, Br) or heteroatom having H or small (C₁-C₂) alkyl substituents. At the 17α position, highly preferred substituents include small alkyl (C₁-C₃) with or without small halogen (F, Cl, Br), alkenyl, alkynyl, aryl or heteroaryl groups without or with small alkyl (C₁-C₃), with or without small halogen (F, Cl, Br), or heteroatom having H or small (C₁-C₂) alkyl substituents.

The illustrations in Scheme 2A are based on a simple estrene or estrane system, but all members of Category I, preferably those shown in Schemes 1A and 1B, may be substituted similarly. It is understood that analogs in which the stereochemistry of various ring junction and fusion positions are inverted from that which is typical in the natural steroids (i.e., 5α, 8β, 9α, 10β, 13β, 14α, 17β) can have similar or enhanced bone anabolic activity. This invention is not bound by any theory, but it is believed that selective substitution may modulate the binding affinity and binding kinetics of the compounds to the estrogen receptor, lower non-specific binding, and/or reduce metabolism.

Preferred Category I ANGELs compounds are estrenediols (e.g., 5(10)-estrenediols, 5(6)-estrenediols and 4-estrenediols), androstenediols (e.g., 5(6)-androstenediols and 4-androstenediols), estranediols (e.g., 5α-estranediols and 5β-estranediols), and androstanediols (e.g., 5α-androstanediols and 5β-androstanediols). Examples of preferred ANGELS compounds include 5(10)-estrene-3α,17α-diol, 5(10)-estrene-3α,17β-diol, 5(10)-estrene-3β,17α-diol, 5(10)-estrene-3β,17β-diol, 5(6)-estrene-3α,17α-diol, 5(6)-estrene-3α,17β-diol, 5(6)-estrene-3β,17α-diol, 5(6)-estrene-3β,17β-diol, 5(6)-androstene-3α,17α-diol, 5(6)-androstene-3α,17β-diol, 5(6)-androstene-3β,17α-diol, 5(6)-androstene-3β,17β-diol, 4-estrene-3α,17α-diol, 4-estrene-3α,17α-diol, 4-estrene-3β,17α-diol, 4-estrene-3β,17β-diol, 4-androstene-3α,17α-diol, 4-androstene-3α,17β-diol, 4-androstene-3β,17α-diol, 4-androstene-3β,17β-diol, estrane-3α,17α-diol, estrane-3α,17β-diol, estrane-3β,17α-diol, estrane-3β,17β-diol, androstane-3α,17α-diol, androstane-3α,17β-diol, androstane-3β,17α-diol, androstane-3β,17β-diol, 5α-estrane-3α,17α-diol, 5α-estrane-3α,17β-diol, 5α-estrane-3β,17α-diol, 5α-estrane-3β,17β-diol, 5α-androstane-3α,17α-diol, 5α-androstane-3α,17β-diol, 5α-androstane-3β,17α-diol, 5α-androstane-3β,17β-diol, 5β-estrane-3α,17α-diol, 5β-estrane-3α,17β-diol, 5β-estrane-3β,17α-diol, 5β-estrane-3β,17β-diol, 5β-androstane-3α,17α-diol, 5β-androstane-3α,17β-diol, 5β-androstane-3β,17α-diol, and 5β-androstane-3β,17β-diol.

Many methods for the synthesis of such substituted compounds are known to those skilled in the art. Preferred examples can be found in the general references on steroid synthesis, noted above, particularly for substitution at the 17α position. For substitution at the 7α and 11β positions, specific reference is made to the following publications: 7α: (French et al., 1993b; Tedesco et al., 1997a) and references cited therein; 11β: (French et al., 1993a; Pomper et al., 1990; Tedesco et al., 1997b) and references cited therein. Examples of syntheses of some members of Category I are shown in Scheme 2C.

Examples of some preferred ANGELS compounds are represented by the following formulas (I) to (IV):

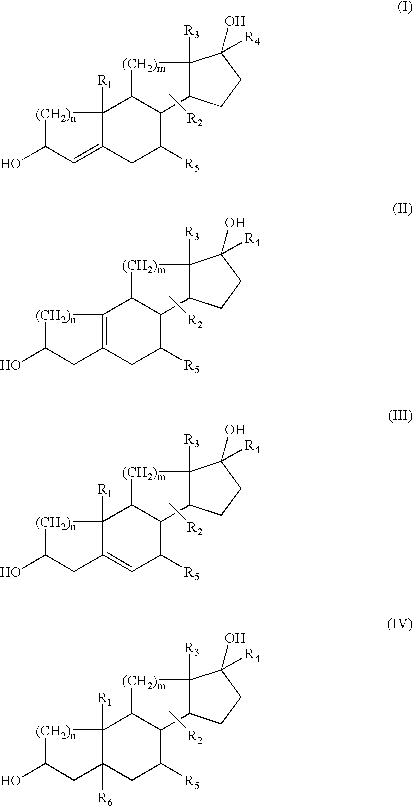

in which R₁, R₃ and R₆ are each individually hydrogen, methyl or ethyl, more preferably methyl; m and n are each individually integers in the range of 1 to 3, R₂ and R₅ are each individually selected from the group consisting of hydrogen, halogen, mercapto, hydroxyl, cyano, amino, ethenyl, ethynyl, aryl, C₁-C₅ heteroaryl, C₁-C₅ alkyl, C₁-C₅ cycloalkyl, C₁-C₅ haloalkyl, C₁-C₅ alkylthio, C₁-C₅ ester, C₁-C₅ alkoxy, C₁-C₅ acyl, C₁-C₅ alkylamine, and C₁-C₅ acyloxy; and R₄ is selected from the group consisting of hydrogen, ethenyl, ethynyl, aryl, C₁-C₅ heteroaryl, C₁-C₅ alkyl, C₁-C₅ cycloalkyl, C₁-C₅ haloalkyl, C₁-C₅ ester, and C₁-C₅ acyl. The chemical structures represented by formulas (I) to (IV) encompass all stereoisomers, and thus the stereochemical configurations of R₁, R₂, R₃, R₄, R₅ and R₆ can each individually be alpha or beta. The R₂ substituent may be attached to any of the (CH₂)ₘ carbon atoms, and/or the other carbons in that ring.

In a preferred embodiment, formula (I) represents 4-estrenediols and 4-androstendediols in which m=n=2 as shown in formula (V) below. Preferably, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl. The structures of preferred 4-estrenediols and 4-androstenediols are described in Table 1 by reference to formula (V).

TABLE 1
4-Estrenediols and 4-Androstenediols

| | |
|---|---|
| | (V) |
| 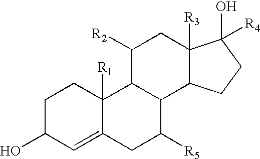 | |

| No. | R₁ | R₂ | R₃ | R₄ | R₅ |
|---|---|---|---|---|---|
| 1 | H | H | Me | H | H |
| 2 | H | H | Me | ethynyl | H |
| 3 | H | H | Me | H | Me |
| 4 | H | H | Me | ethynyl | Me |
| 5 | H | Et | Me | H | H |
| 6 | H | Et | Me | ethynyl | H |
| 7 | H | Et | Me | H | Me |
| 8 | H | Et | Me | ethynyl | Me |
| 9 | Me | H | Me | H | H |
| 10 | Me | H | Me | ethynyl | H |
| 11 | Me | H | Me | H | Me |
| 12 | Me | H | Me | ethynyl | Me |
| 13 | Me | Et | Me | H | H |
| 14 | Me | Et | Me | ethynyl | H |
| 15 | Me | Et | Me | H | Me |
| 16 | Me | Et | Me | ethynyl | Me |

In a preferred embodiment, formula (II) represents 5(10)estrenediols in which m=n=2 as shown in formula (VI) below. Preferably, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl. The structures of preferred 5(10)estrenediols are described in Table 2 by reference to formula (VI).

TABLE 2
5(10) Estrenediols

| | |
|---|---|
| | (VI) |
| 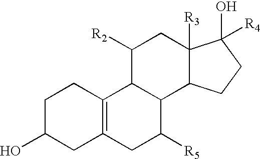 | |

| No. | R₂ | R₃ | R₄ | R₅ |
|---|---|---|---|---|
| 1 | H | Me | H | H |
| 2 | H | Me | ethynyl | H |
| 3 | H | Me | H | Me |
| 4 | H | Me | ethynyl | Me |
| 5 | Et | Me | H | H |
| 6 | Et | Me | ethynyl | H |
| 7 | Et | Me | H | Me |
| 8 | Et | Me | ethynyl | Me |

In a preferred embodiment, formula (III) represents 5(6)estrenediols and 5(6)androstenediols in which m=n=2 as shown in formula (VII) below. Preferably, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl. The structures of preferred 5(6)estrenediols and 5(6)androstenediols are described in Table 3 by reference to formula (VII).

TABLE 3
5(6)Estrenediols and 5(6)Androstenediols

| | |
|---|---|
| | (VII) |
| 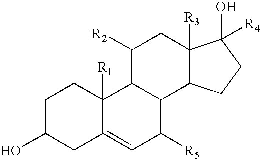 | |

| No. | R₁ | R₂ | R₃ | R₄ | R₅ |
|---|---|---|---|---|---|
| 1 | H | H | Me | H | H |
| 2 | H | H | Me | ethynyl | H |
| 3 | H | H | Me | H | Me |
| 4 | H | H | Me | ethynyl | Me |
| 5 | H | Et | Me | H | H |
| 6 | H | Et | Me | ethynyl | H |
| 7 | H | Et | Me | H | Me |
| 8 | H | Et | Me | ethynyl | Me |
| 9 | Me | H | Me | H | H |
| 10 | Me | H | Me | ethynyl | H |
| 11 | Me | H | Me | H | Me |
| 12 | Me | H | Me | ethynyl | Me |
| 13 | Me | Et | Me | H | H |
| 14 | Me | Et | Me | ethynyl | H |
| 15 | Me | Et | Me | H | Me |
| 16 | Me | Et | Me | ethynyl | Me |

In a preferred embodiment, formula (IV) represents estranediols and androstanediols in which m=n=2 as shown in formula (VIII) below. The structures of preferred estranediols and androstanediols in which R₆ is hydrogen are described in Table 4 by reference to formula (VIII). Preferably, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl. Those skilled in the art will appreciate that formula (VIII) represents all stereoisomers, including the 5α and 5β stereoisomers.

TABLE 4
Estranediols and Androstanediols

| | |
|---|---|
| | (VIII) |
| 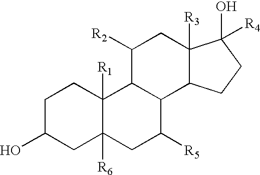 | |

| No. | R₁ | R₂ | R₃ | R₄ | R₅ |
|---|---|---|---|---|---|
| 1 | H | H | Me | H | H |
| 2 | H | H | Me | ethynyl | H |
| 3 | H | H | Me | H | Me |
| 4 | H | H | Me | ethynyl | Me |
| 5 | H | Et | Me | H | H |
| 6 | H | Et | Me | ethynyl | H |
| 7 | H | Et | Me | H | Me |
| 8 | H | Et | Me | ethynyl | Me |
| 9 | Me | H | Me | H | H |
| 10 | Me | H | Me | ethynyl | H |
| 11 | Me | H | Me | H | Me |
| 12 | Me | H | Me | ethynyl | Me |
| 13 | Me | Et | Me | H | H |
| 14 | Me | Et | Me | ethynyl | H |
| 15 | Me | Et | Me | H | Me |
| 16 | Me | Et | Me | ethynyl | Me |

Category II: Ring-Modified Analogs of Estrenes and Estranes

Many estrenediol, androstenediol, estranediol and androstanediol analogs are known in which the sizes of the rings are enlarged (termed A, B, C or D ring “homoestrenediols, homoandrostenediols, homoestranediols and homoandrostanediols”), contracted (termed A, B, C or D ring “norestrenediols, norandrostenediols, norestranediols and norandrostanediols”), or broken (termed A, B, C, or D ring “secoestrenediols, secoandrostenediols, secoestranediols and secoandrostanediols” or A/B, B/C, or C/D “cycloestrenediols, cycloandrostenediols, cycloestranediols and cycloandrostanediols”). Examples of compounds in Category II are shown in Scheme 3A, and a general structure that shows preferred ring sizes, substituents, and substitution patterns is shown in Scheme 3B.

The examples illustrated in Schemes 3A and 3B are based on one typical estrene, but Category II also includes the analogous homo-, nor-, seco-, and cyclo- analogs of any of the estrene, estrane, androstene, or androstane analogs indicated in Schemes 1A and 1B. Category II also includes analogs in which the stereochemistry of various ring junction and fusion positions are inverted from that which is typical in the natural steroids (i.e., 5α, 8β, 9α, 10β, 13β, 14α, 17β).

Many methods are available for the synthesis of homo-, nor-, seco-, and cyclo-analogs. Many examples can be found in the general references on steroid synthesis, noted above. Specific reference is made to the following additional publications on steroid synthesis and modification reactions: Fieser and Fieser, 1967; Fried and Edwards, 1972; Kirk and Hartshorn, 1968; Shoppee, 1964; Djerassi, 1963; and references cited therein. In addition, there are many general known methods for the enlargement of carbocyclic rings, as needed to prepare the homo-steroids, for the contraction of carbocyclic rings as needed to prepare the nor-steroids, as well as for the cleavage of carbocyclic rings and carbon chains, as needed to obtain the various seco- and cyclo-analogs (Paquette, 1995; Trost, 1991). These methods are well described in general books on synthetic methodology, such as the books by Smith and March (Smith and March, 2001) and Larock (Larock, 1989), as well as in review articles on these specific topics. Examples of syntheses of some members of Category II are shown in Scheme 3C.

In a preferred embodiment, formula (I) represents nor-estrenediols and nor-androstenediols in which m and/or n are 1 or 2, homo-estrenediols and homo-androstenediols in which m and/or n are 2 or 3, or estrenediols and androstenediols containing both nor- and homo-rings in which one of m or n is 1 and the other is 3. The structures of various preferred ANGELS compounds in which R₂ and R₅ are hydrogen and R₃ in Table 5 by reference to formula (I). is methyl are described in Table 5 by reference to formula (I).

TABLE 5
Nor/homo-estrenediols and nor/homo-androstenediols

| No. | n | m | R₁ | R₄ |
|---|---|---|---|---|
| 1 | 1 | 2 | H | H |
| 2 | 1 | 2 | H | ethynyl |
| 3 | 1 | 2 | Me | H |
| 4 | 1 | 2 | Me | ethynyl |
| 5 | 2 | 1 | H | H |
| 6 | 2 | 1 | H | ethynyl |
| 7 | 2 | 1 | Me | H |
| 8 | 2 | 1 | Me | ethynyl |
| 9 | 3 | 2 | H | H |
| 10 | 3 | 2 | H | ethynyl |
| 11 | 3 | 2 | Me | H |
| 12 | 3 | 2 | Me | ethynyl |
| 13 | 2 | 3 | H | H |
| 14 | 2 | 3 | H | ethynyl |
| 15 | 2 | 3 | Me | H |
| 16 | 2 | 3 | Me | ethynyl |

In a preferred embodiment, formula (II) represents nor-5(10)-estrenediols in which m and/or n are 1 or 2, homo-5(10)-estrenediols in which m and/or n are 2 or 3, and 5(10)-estrenediols containing both nor- and homo-rings in which one of m or n is 1 and the other is 3. Preferably, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen, C₁-C₅ alkyl. The structures of various preferred ANGELS compounds in which R₃ is methyl and R₅ is hydrogen are described in Table 6 by reference to formula (II).

TABLE 6
Nor/homo-estrenediols

| No. | n | m | R₂ | R₄ |
|---|---|---|---|---|
| 1 | 1 | 2 | H | H |
| 2 | 1 | 2 | H | ethynyl |
| 3 | 1 | 2 | Me | H |
| 4 | 1 | 2 | Me | ethynyl |
| 5 | 2 | 1 | H | H |
| 6 | 2 | 1 | H | ethynyl |
| 7 | 2 | 1 | Me | H |
| 8 | 2 | 1 | Me | ethynyl |
| 9 | 3 | 2 | H | H |
| 10 | 3 | 2 | H | ethynyl |
| 11 | 3 | 2 | Me | H |
| 12 | 3 | 2 | Me | ethynyl |
| 13 | 2 | 3 | H | H |
| 14 | 2 | 3 | H | ethynyl |
| 15 | 2 | 3 | Me | H |
| 16 | 2 | 3 | Me | ethynyl |

In a preferred embodiment, formula (III) represents nor-5(6)-estrenediols and nor-5(6)-androstenediols in which m and/or n are 1 or 2, homo-5(6)-estrenediols and homo-5(6)-androstenediols in which m and/or n are 2 or 3, and 5(6)-estrenediols and 5(6)-androstenediols containing both nor- and homo-rings in which one of m or n is 1 and the other is 3. Preferably, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl. The structures of preferred ANGELS compounds in which R₃ is methyl and R₅ is hydrogen are described in Table 7 by reference to formula (III).

TABLE 7
Nor/homo-estrenediols and nor/homo-androstenediols

| No. | n | m | R₁ | R₂ | R₄ |
|---|---|---|---|---|---|
| 1 | 1 | 2 | H | H | H |
| 2 | 1 | 2 | H | H | ethynyl |
| 3 | 1 | 2 | Me | H | H |
| 4 | 1 | 2 | Me | H | ethynyl |
| 5 | 2 | 1 | H | H | H |
| 6 | 2 | 1 | H | H | ethynyl |
| 7 | 2 | 1 | Me | H | H |
| 8 | 2 | 1 | Me | H | ethynyl |
| 9 | 3 | 2 | H | Me | H |
| 10 | 3 | 2 | H | Me | ethynyl |
| 11 | 3 | 2 | Me | Me | H |
| 12 | 3 | 2 | Me | Me | ethynyl |
| 13 | 2 | 3 | H | Me | H |
| 14 | 2 | 3 | H | Me | ethynyl |
| 15 | 2 | 3 | Me | Me | H |
| 16 | 2 | 3 | Me | Me | ethynyl |

In a preferred embodiment, formula (IV) represents nor-estranediols and nor-androstanediols in which m and/or n are 1 or 2, homo-estranediols and homo-androstanediols in which m and/or n are 2 or 3, and estranediols and androstanediols containing both nor- and homo-rings in which one of m or n is 1 and the other is 3. Preferably, R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl. The structures of preferred ANGELS compounds in which R₂ and R₅ are hydrogen and R₃ is methyl are described in Table 8 by reference to formula (IV).

TABLE 8
Nor/homo-estranediols and nor/homo-androstanediols

| No. | n | m | R₁ | R₄ |
|---|---|---|---|---|
| 1 | 1 | 2 | H | H |
| 2 | 1 | 2 | H | ethynyl |
| 3 | 1 | 2 | Me | H |
| 4 | 1 | 2 | Me | ethynyl |
| 5 | 2 | 1 | H | H |
| 6 | 2 | 1 | H | ethynyl |
| 7 | 2 | 1 | Me | H |
| 8 | 2 | 1 | Me | ethynyl |
| 9 | 3 | 2 | H | H |
| 10 | 3 | 2 | H | ethynyl |
| 11 | 3 | 2 | Me | H |
| 12 | 3 | 2 | Me | ethynyl |
| 13 | 2 | 3 | H | H |
| 14 | 2 | 3 | H | ethynyl |
| 15 | 2 | 3 | Me | H |
| 16 | 2 | 3 | Me | ethynyl |

In preferred embodiments, ANGELS compounds of Category (III) are represented by the following structures wherein R₁₃, R₁₄, and R₁₅ are each individually selected from the group consisting of hydrogen, ethenyl, ethynyl, C₁-C₅ alkyl, cycloalkyl and phenyl; and wherein R₁₆ is selected from the group consisting of hydrogen, hydroxyl, and C₁-C₅ hydroxyalkyl.

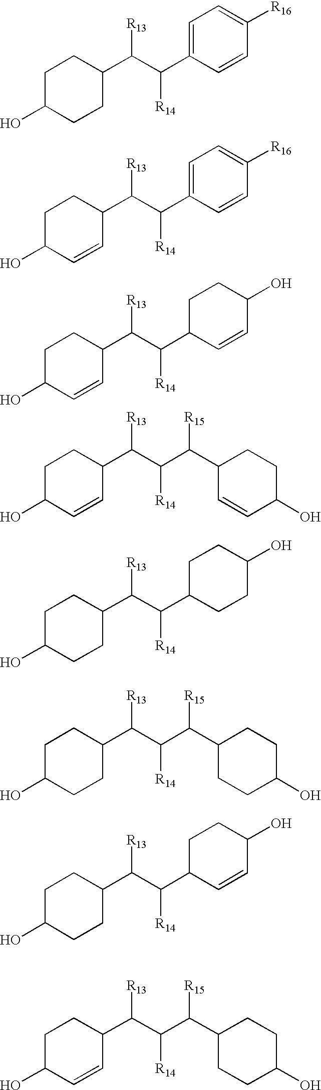

More preferably, in each of the structures shown immediately above, R₁₃, R₁₄ and R₁₅ are each individually selected from the group consisting of hydrogen, C₁-C₅ alkyl, cycloalkyl and phenyl; and R₁₆ is preferably hydroxyl. For this embodiment, the more preferred structures are represented by a formula selected from the group consisting of

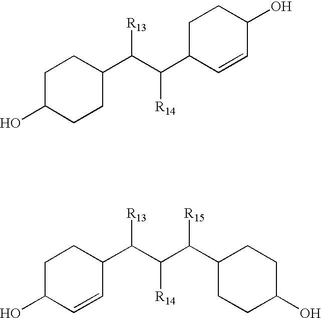

Most preferably, in each of the structures shown immediately above, R₁₃, R₁₄ and R₁₅ are each individually selected from the group consisting of hydrogen, C₁-C₅ alkyl, cycloalkyl and phenyl.

In preferred embodiments, ANGELS compounds of Category (II) are represented by the following structures, in which m and n are each individually integers in the range of 1 to 4; R₃ and R₅ are each individually selected from the group consisting of hydroxy, hydrogen, C₁ to C₅ alkyl, C₁ to C₅ hydroxyalkyl, C₁ to C₅ alkoxy, C₁ to C₅ thioalkoxy, phenyl, and C₁ to C₅ alkyl-substituted phenyl; and in which R₆ is selected from the group consisting of hydrogen and C₁-C₅ alkyl:

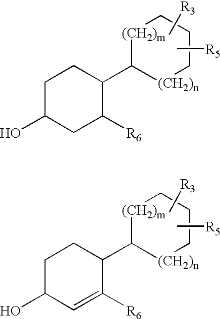

More preferred ANGELS compounds in this preferred embodiment have a structure selected from the following group, in which R₃, R₅ and R₆ each have the same meaning as described above:

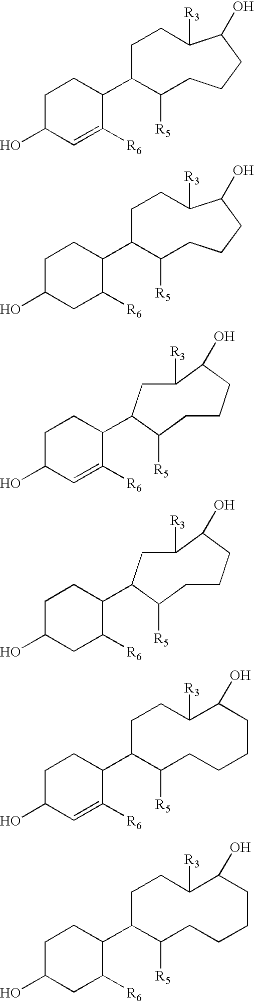

Most preferably, in each of the structures shown immediately above, R₃ is selected from the group consisting of hydrogen, methyl and ethyl; and R₅ and R₆ are each individually selected from the group consisting of hydrogen and C₁-C₅ alkyl.

Category III: Heterocyclic and Heteroacyclic Analogs of Estrene and Estrane

Preferred members of Category III are shown Scheme 4A; general structures are shown in Scheme 4B. The illustrated structures are based on a simple estrene or estrane system, but heterocyclic and heteroacyclic analogs of other estrenes, estranes, androstenes and androstanes such as shown in Scheme 1 are included in Category III.

The heteroatoms in the Category III compounds may facilitate rapid synthesis by allowing the use of combinatorial synthetic methods that are easily adapted to solid phase or solution phase automated synthesis methods, see, e.g., Stauffer and Katzenellenbogen, 2000b and references cited therein.

The synthesis of compounds in Category III can be accomplished by methods that are described in the above-cited references, as well as basic heterocyclic synthesis methods, as described in various books on this topic (Eieher and Hauptmann, 1995; Gilchrist, 1992; Gupta et al., 1999; Joule et al., 1995), and references cited therein, as well as by using basic heteroatom-based synthesis methods that are well known to those skilled in the art of organic synthesis. Examples of syntheses of some members of this class are illustrated in Schemes 4C and 4D.

ANGELS compounds may also be heterocyclic estrene analogs. Various preferred heterocyclic estrene analogs may be represented by the following formulas, in which R is hydrogen or C₁-C₅ alkyl; and in which R′ and R″ are each individually selected from the group consisting of hydrogen, C₁-C₅ alkyl, trifluoromethyl, and C₁-C₅ alkyl-substituted phenyl. Examples of preferred ANGELS compounds are described in Table 9 below.

TABLE 9
Heterocyclic Estrene Analogs

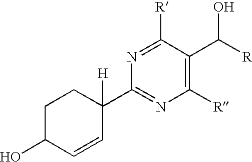

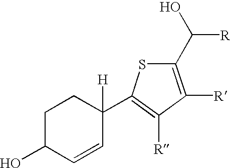

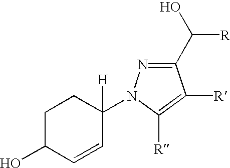

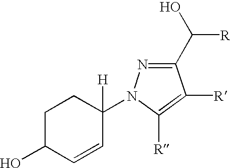

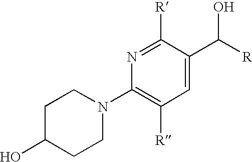

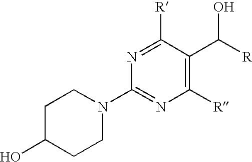

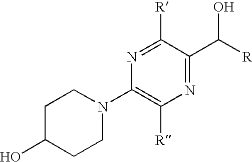

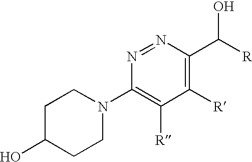

| No. | R | R′ | R″ |
|---|---|---|---|
| 1 | H | H | H |
| 2 | H | H | H, Me, Et, Pr, i-Pr |
| 3 | H | H | CF₃ |
| 4 | H | H | Ph |
| 5 | H | H | o-Tol, m-Tol, p-Tol |
| 6 | H | Me | H, Me, Et, Pr, i-Pr |
| 7 | H | Me | CF₃ |
| 8 | H | Me | Ph |
| 9 | H | Me | o-Tol, m-Tol, p-Tol |
| 10 | Me | Me | H, Me, Et, Pr, i-Pr |
| 11 | Me | Me | CF₃ |
| 12 | Me | Me | Ph |
| 14 | Me | Me | o-Tol, m-Tol, p-Tol |
| 15 | Me | H, Me, Et, Pr, i-Pr | H, Me, Et, Pr, i-Pr |
| 16 | Me | CF₃ | CF₃ |
| 17 | Me | Ph | Ph |
| 18 | Me | o-Tol, m-Tol, p-Tol | o-Tol, m-Tol, p-Tol |
| 19 | H, Me, Et, Pr, i-Pr | Me | Me |
| 20 | H, Me, Et, Pr, i-Pr | Et | Et |
| 21 | H, Me, Et, Pr, i-Pr | Pr | Pr |
| 22 | H, Me, Et, Pr, i-Pr | Ph | Ph |
| 23 | H, Me, Et, Pr, i-Pr | CF₃ | CF₃ |
| 24 | H, Me, Et, Pr, i-Pr | o-Tol, m-Tol, p-Tol | o-Tol, m-Tol, p-Tol |

ANGELS compounds may also be heteroacyclic estrene analogs. Various preferred heteroacyclic estrene analogs may be represented by the following formulas, in which R₁ is selected from the group consisting of hydrogen, C₁-C₅ alkyl, cycloalkyl, phenyl, and C₁-C₅ alkyl phenyl; R₂ is selected from the group consisting of hydrogen, C₁-C₅ alkyl, and trifluoromethyl; and R₃ is selected from the group consisting of hydrogen, C₁-C₅ alkyl, cycloalkyl, hydroxycycloalkyl, phenyl, and C₁-C₅ alkyl phenyl. Examples of preferred ANGELS compounds are described in Table 10 below.

TABLE 10
Heteroacyclic Estrene Analogs

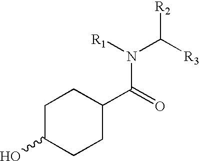

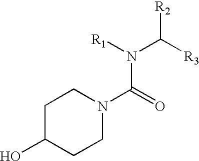

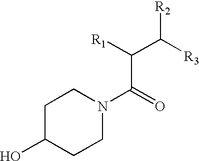

| No. | R₁ | R₂ | R |
|---|---|---|---|
| 1 | Cyclohexyl | CF₃ | 4-hydroxycyclohexyl |
| 2 | Cyclohexyl | CF₃ | i-Pr |
| 3 | Cyclohexyl | CF₃ | cyclohexyl |
| 4 | Cyclohexyl | CF₃ | Ph |
| 5 | phenyl | CF₃ | 4-hydroxycyclohexyl |
| 6 | phenyl | CF₃ | i-Pr |
| 7 | phenyl | CF₃ | cyclohexyl |
| 8 | phenyl | CF₃ | Ph |
| 9 | Cyclohexyl | Me, Et, Pr, i-Pr | 4-hydroxycyclohexyl |
| 10 | Cyclohexyl | Me, Et, Pr, i-Pr | i-Pr |
| 11 | Cyclohexyl | Me, Et, Pr, i-Pr | cyclohexyl |
| 12 | Cyclohexyl | Me, Et, Pr, i-Pr | Ph |
| 13 | phenyl | Me, Et, Pr, i-Pr | 4-hydroxycyclohexyl |
| 14 | phenyl | Me, Et, Pr, i-Pr | i-Pr |
| 15 | phenyl | Me, Et, Pr, i-Pr | cyclohexyl |
| 16 | phenyl | Me, Et, Pr, i-Pr | Ph |

Category IV: Estren-3-ol Analogs

Category IV includes analogs of estren-3-ol, e.g. estratrienol analogs. Various preferred examples of compounds in Category IV are illustrated in Scheme 5A, and a general structure describing Category IV compounds is illustrated in Scheme 5B. The basic design of these compounds preferably involves an estrogen-like A-ring, that is a phenol, having the hydroxyl group at either the C-1, 2, 3, or 4 position, or various combinations thereof, with the remainder of the structure being selected to achieve maximum potency and efficacy.

Like the estrene analogs described in Schemes 1-4, the estratrienols can embody various analogous structures in the B, C, and D rings, including substituents that enhance efficacy and/or selectivity (as specified in Scheme 2A), nor-, homo-, seco-, and cyclo-steroid analogs (as specified in Schemes 3A and B), and heterocyclic and heteroacyclic analogs (as specified in Schemes 4A and B). Category IV includes these analogs.

For synthesis purposes, estratrienols tend to be more like estrogens than are estrenes, and their syntheses can utilize the general and specific synthetic methodologies noted above for the estrenes, with suitable modifications to accommodate the estratrienol functionality in the A-ring. Such modifications are known to those skilled in the art of steroid synthesis. Examples of syntheses of some Category IV compounds are illustrated in Schemes 5C and 5D.

Pharmaceutical Compositions Comprising ANGELS Compounds

A preferred embodiment provides pharmaceutical compositions comprising one or more ANGELS compounds, preferably one or more compounds of Category I, II, III, and/or IV. Thus, an ANGELS compound or mixture thereof can be administered in an amount effective to increase bone mass and/or density and/or strength as described herein, optionally in admixture with a pharmaceutically acceptable carrier or diluent as described below. It is understood that the description herein of various ways of administering the ANGELS compounds disclosed herein applies to pharmaceutical compositions comprised of those compounds.

ANGELS compounds can be administered by any appropriate route for systemic, local or topical delivery, for example, orally, parenterally, intravenously, intradermally, subcutaneously, buccal, intranasal, inhalation, vaginal, rectal or topically, in liquid or solid form. Methods of administering the compounds described herein may be by specific dose or by controlled release vehicles.

A preferred mode of administration of the ANGELS compounds is oral. Oral compositions preferably include an inert diluent or an edible carrier. The active compound can be enclosed in gelatin capsules or compressed into tablets. For the purpose of oral therapeutic administration, the compound can be incorporated with excipients and used in the form of tablets, troches, or capsules. Pharmaceutically compatible binding agents, and/or adjuvant materials can be included as part of the composition.

The tablets, pills, capsules, troches and the like can contain any of the following pharmaceutically acceptable carriers, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; and/or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The ANGELS compound can be administered as a component of an elixir, suspension, syrup, wafer, chewing gum or the like. A syrup may contain, in addition to the active compounds, sucrose as a sweetening agent and certain preservatives, dyes and colorings and flavors.

The ANGELS compound can also be mixed with other active materials that do not impair the desired action, or with materials that supplement the desired action, such as one or more other ANGELS compounds; classical estrogens like 17β-estradiol or ethinyl estradiol; bisphosphonates like alendronate, etidronate, pamidronate, risedronate, tiludronate, zoledronate, cimadronate, clodronate, ibandronate, olpadronate, neridronate, EB-1053; calcitonin of salmon, eel or human origin; and anti-oxidants like glutathione, ascorbic acid or sodium bisulfite. Pharmaceutically acceptable carriers can be solutions or suspensions used for parenteral, intradermal, subcutaneous, or topical application, and thus may comprise one or more of the following components: a sterile diluent such as water for injection, saline solution, fixed oils, polyethylene glycols, glycerine, propylene glycol or other synthetic solvents; antibacterial agents such as benzyl alcohol or methyl parabens; chelating agents such as ethylenediaminetetraacetic acid (EDTA); buffers such as acetates, citrates or phosphates and agents for the adjustment of tonicity such as sodium chloride or dextrose. The parental preparation can be enclosed in ampoules, disposable syringes or multiple dose vials made of glass or plastic. If administered intravenously, preferred carriers are physiological saline or phosphate buffered saline (PBS).

In a preferred embodiment, the ANGELS compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations are known to those skilled in the art.

Liposomal suspensions (including liposomes targeted with monoclonal antibodies to surface antigens of specific cells) are also pharmaceutically acceptable carriers. These may be prepared according to methods known to those skilled in the art, for example, as described in U.S. Pat. No. 4,522,811. For example, liposome formulations may be prepared by dissolving appropriate lipid(s) (such as stearoyl phosphatidyl ethanolamine, stearoyl phosphatidyl choline, arachadoyl phosphatidyl choline, and/or cholesterol) in an inorganic solvent that is then evaporated, leaving behind a thin film of dried lipid on the surface of the container. An aqueous solution of the ANGELS compound or its monophosphate, diphosphate, and/or triphosphate derivative(s) is then introduced into the container. The container is then swirled by hand to free lipid material from the sides of the container and to disperse lipid aggregates, thereby forming the liposomal suspension.

For parenteral administration, the ANGELS compound is preferably formulated in a unit dosage injectable form (solution, suspension, emulsion) in association with a pharmaceutically acceptable carrier that is a parenteral vehicle. Such vehicles are preferably non-toxic and non-therapeutic. Examples of such vehicles are water, saline, Ringer's solution, dextrose solution, and 5% human serum albumin. Nonaqueous vehicles such as fixed oils and ethyl oleate may also be used. Liposomes may be used as carriers. The vehicle may contain minor amounts of additives such as substances that enhance isotonicity and chemical stability, e.g., buffers and preservatives. ANGELS compounds are preferably formulated in such vehicles at concentrations of about 10 nanograms/ml to about 100 milligrams/ml, more preferably 10 micrograms/ml to about 10 milligrams/ml.

The concentration of the ANGELS compound in the pharmaceutical composition is preferably adjusted by taking into account the absorption, inactivation, and excretion rates of the compound as well as other factors known to those of skill in the art.

Methods of Treatment Using ANGELS Compounds and Pharmaceutical Compositions Thereof

The ANGELS compounds disclosed herein (including pharmaceutical compositions comprising these compounds) are preferably used to treat mammals, more preferably humans. A preferred method of treatment involves identifying a mammal in need of treatment and administering a therapeutically effective amount of one or more ANGELS compounds, more preferably one or more compounds in Categories I, II, III, and/or IV to the mammal.

The ANGELS compounds described herein are useful for maintaining and/or increasing bone mass and/or density and/or strength. Preferably, the ANGELS compounds described herein are used to treat individuals identified as having low bone mass and/or density and/or strength, and/or individuals at risk of developing low bone mass and/or density and/or strength. Methods for identifying mammals having low bone mass and/or density and/or strength are known to those skilled in the art and include dual energy absorptiometry, clinical bone sonometry, X-rays, CAT scans, and histomorphometric examination of bone biopsies. Symptoms of bone loss can include back pain, loss of height over time, with accompanying stooped posture, and increasing frequency of bone fractures. Methods for identifying mammals at risk of developing low bone mass and/or density and/or strength are also known to those skilled in the art and include assessment of various risk factors such as gender, age, race, family history, tobacco use, estrogen or androgen deficiency, exposure to corticosteroids, and chronic alcoholism.

The ANGELS compounds described herein are useful for other indications, such as to increase libido, control vasomotor disturbance, promote vasodilation, reduce bone loss, reduce mood swings, lower cholesterol, decrease low density lipoproteins (LDL), increase high density lipoproteins (HDL), slow atherosclerosis, slow progression of cancer, slow progression of cardiovascular disease, slow age-related neurodegeneration, slow progression of neurodegenerative disease, reduce risk of cancer, reduce risk of cardiovascular disease, reduce risk of stroke, and/or reduce risk of neurodegencrative disease.

The ANGELS compounds disclosed herein (including pharmaceutical compositions comprising these compounds) are preferably administered to mammals by dosage regimens that provide the compounds to the mammals in therapeutically effective amounts. A therapeutically effective amount can be an amount that is effective to slow the rate of loss of bone mass and/or density and/or strength, but is preferably an amount that is effective to maintain and/or increase mass and/or density and/or strength.

Preferred therapeutically effective amounts can vary over a broad range. The dose and dosage regimen is preferably selected by considering the nature of the patient's need for treatment, e.g., need for an increase in bone density and/or strength, the characteristics of the particular active ANGELS compound, e.g., its therapeutic index, the patient, the patient's history and other factors known to those skilled in the art. Preferred daily dosages of ANGELS compound are typically in the range of about 1 microgram/kg to about 100 milligrams/kg of patient weight, although higher or lower doses may be used in appropriate circumstances. More preferably, daily dosages of ANGELS compound are typically in the range of about 10 micrograms/kg to about 10 milligrams/kg of patient weight, or an equivalent sustained release dosage. A preferred dosage regimen includes administering the ANGELS compound to the subject over an extended period of time, preferably for at least about 1 month, more preferably at least about 3 months.

Therapeutically effective amounts can be determined by those skilled in the art by such methods as clinical trials. Dosage may be adjusted in individual cases as required to achieve the desired maintenance and/or increase in bone mass and/or density and/or strength. Sustained release dosages and infusions are specifically contemplated. Administration may be oral, by inhalation, by injection, by infusion, by implantation, or by any other suitable route.

The ANGELS compound may be administered at once, or may be divided into a number of smaller doses to be administered at varying intervals of time. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the claimed invention.

This invention is not bound by any theory of operation. Accordingly, the following discussion of the mechanism by which the ANGELS compounds are believed to act is provided for the benefit of those skilled in the art, but does not limit the scope of the invention.

It is believed that estrogens and androgens exert their regulatory influences on many tissues and organs by signaling through highly specialized proteins that belong to the superfamily of nuclear receptors: the estrogen receptors (ERs) α and β and the androgen receptor (AR), respectively (King and Greene, 1984; Quigley et al, 1995; Mangelsdorf et al, 1995; Kuiper et al, 1996; McKenna and O'Malley, 2002; Katzenellenbogen et al, 1996; Moggs and Orphamides, 2001; Hall et al, 2001). Nonetheless, numerous effects of these hormones cannot be explained by the established models of transcriptional regulation resulting from cis- or trans-interactions of the receptor with DNA. Such effects have been collectively attributed to “nongenomic” or “non-genotropic” actions (Pietras and Szego, 1977; Valverde et al, 1999; McEwen and Alves, 1999; Toran-Allerand et al, 1999; Chen et al, 1999; Simoncini et al, 2000; Falkenstein et al, 2000; Wyckoff et al, 2001; Levin, 2001). The relationship of “non-genotropic” actions to the better known effects of sex steroids on transcription remains largely unknown. Moreover, heretofore, there has been no evidence that non-genotropic actions of sex steroids are of biological relevance in vivo.

It has been recently demonstrated that estrogens and androgens attenuate the apoptosis of osteoblasts/osteocytes by rapidly activating a Src/Shc/ERK signaling pathway (Kousteni et al, 2001). This effect requires only the ligand binding domain of their receptors and unlike the classical genotropic action of the receptor proteins is eliminated by nuclear targeting. Unexpectedly, ERα, ERβ or AR mediate this effect with similar efficiency irrespective of whether the ligand is an estrogen or an androgen. Moreover, this non-genotropic effect can be dissociated from the genotropic actions of the receptors with synthetic ligands.

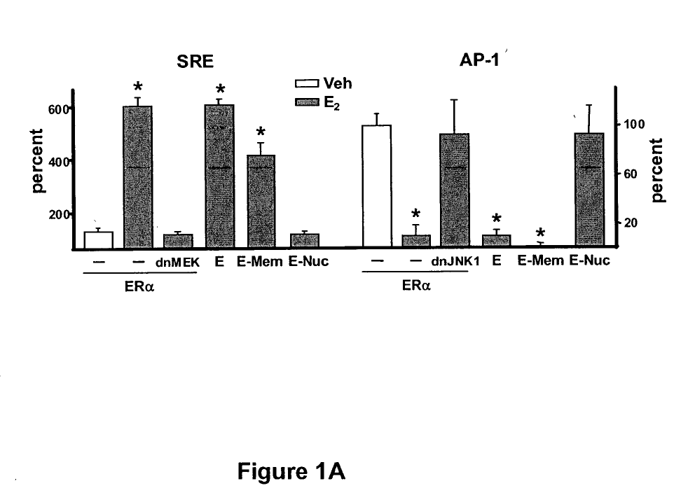

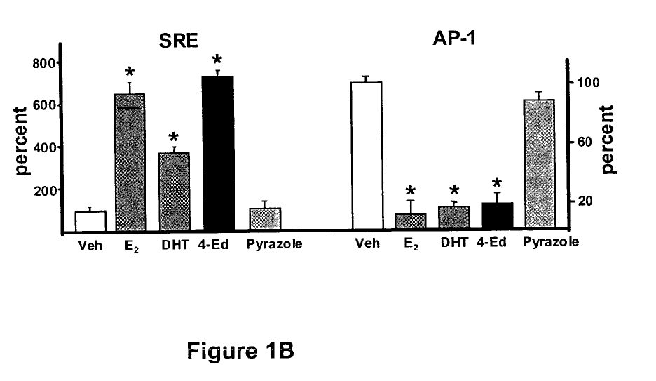

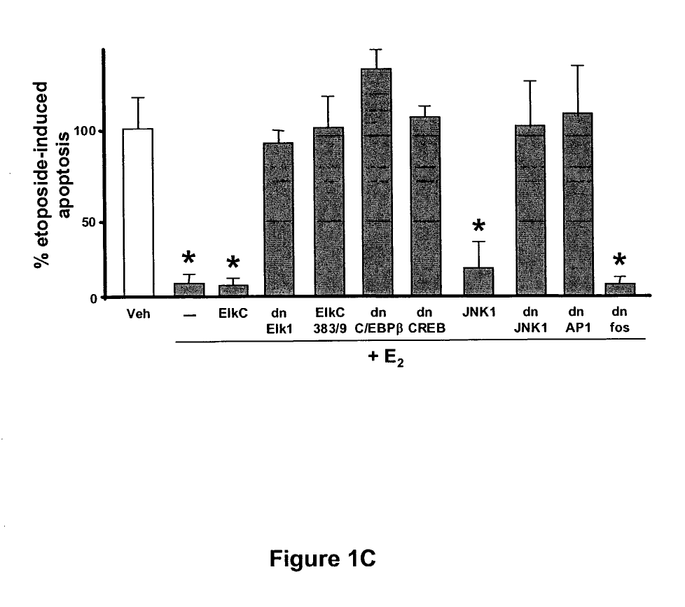

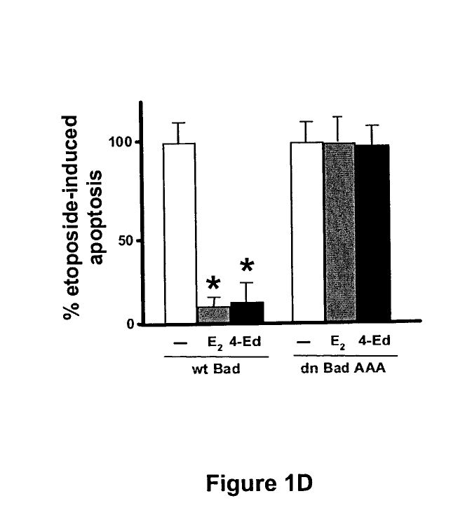

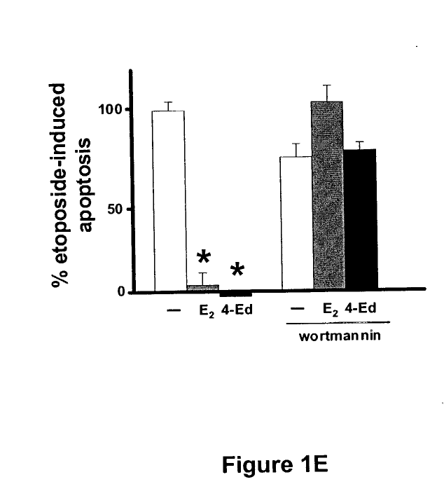

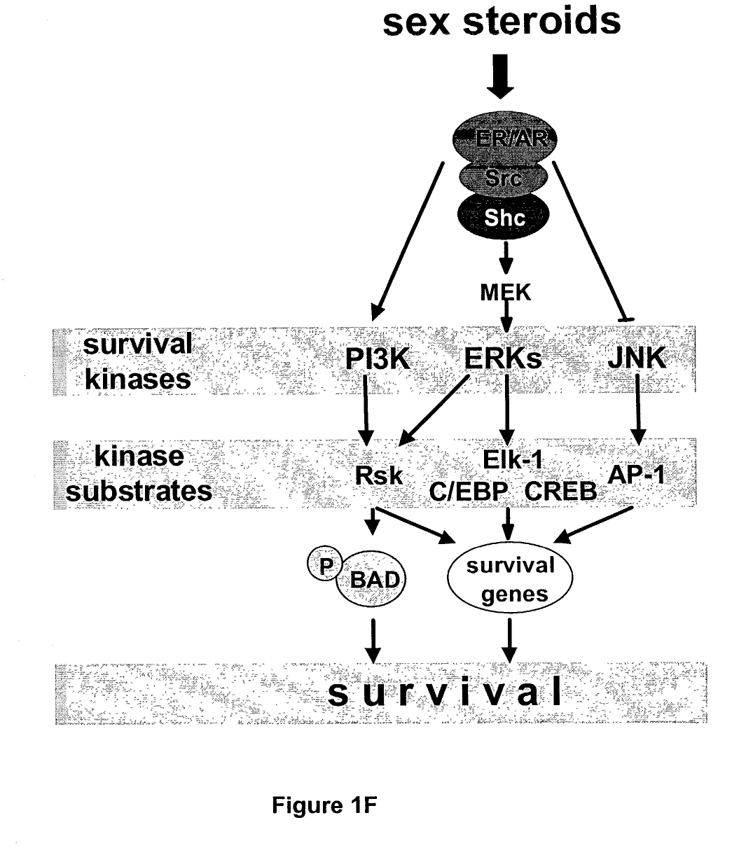

It has now been discovered that sex steroids protect the adult skeleton through a fundamentally distinct mechanism of receptor action than that utilized to preserve the mass and function of reproductive organs. Specifically, the results described herein demonstrate that estrogens or androgens exert anti-apoptotic effects on osteoblasts and pro-apoptotic effects on osteoclasts through a non-genotropic regulation of MAP kinases and downstream transcription-dependent and -independent events. These actions display relaxed ligand/receptor specificity consistent with the demonstration of an equivalence of the bone, but not reproductive, effects of sex steroids in female and male mice. A preferred compound of the invention, 4-estren-3α,17β-diol, faithfully reproduces these non-genotropic effects without affecting classical transcription, increases bone mass in ovariectomized females above the level of the estrogen replete state, and is at least as effective as DHT in orchidectomized males, without affecting reproductive organs in either sex, thus avoiding or minimizing the side effects and risks associated with use of estrogens or androgens. These findings indicate that ANGELS compounds represent a new class of pharmacotherapeutics with the potential for a bone anabolic, sex neutral, hormone replacement therapy. These and additional data are depicted in FIGS. 1-15.

FIG. 1 demonstrates that non-genotropic activation of cytoplasmic kinases and downstream transcription-dependent and -independent events are required for the anti-apoptotic effects of sex steroids. HeLa cells were co-transfected with reporter constructs in which SRE or AP-1 drive the expression of secreted alkaline phosphatase (SEAP), along with the wild type ERα (A and B); or its ligand binding domain (E), or E fused to a membrane (E-Mem) or nuclear (E-Nuc) localization sequence (A). A dominant negaitve (dn) MEK or dn Jnk were also introduced into a subset of the ERα transfected cells. Cells were exposed to vehicle or the indicated steroids (10⁻⁸M) for 15 minutes. The steroid containing media were then removed, the cells were washed twice, and the cultures were continued in fresh medium without steroids. Supernatants were collected six hours later and SEAP activity was assayed. In FIGS. 1A and 1B, 100% indicates activity in vehicle treated cells. (C) HeLa cells were co-transfected with the ERα and nEGFP and wild type or dn mutants of the indicated transcription factors. Transfected cells were treated for 1 h with 10⁻⁸ M E₂ followed by 6 h treatment with etoposide (100 μM) and apoptosis was assayed by nuclear morphology of fluorescent cells. (D) HeLa cells (upper panel) were transiently co-transfected with the ERα, a nonphosphorylatable dn mutant of Bad. Calvaria-derived murine osteoblastic cells (lower panel) were pre-treated with the P13K inhibitor wortmannin. Cells were then treated as in (C). Bars indicate means±SD of triplicate determinations, *p<0.05 vs. vehicle, by ANOVA. (E) Proposed model for a kinase-mediated non-genotropic regulation of gene transcription and apoptosis by sex steroids.

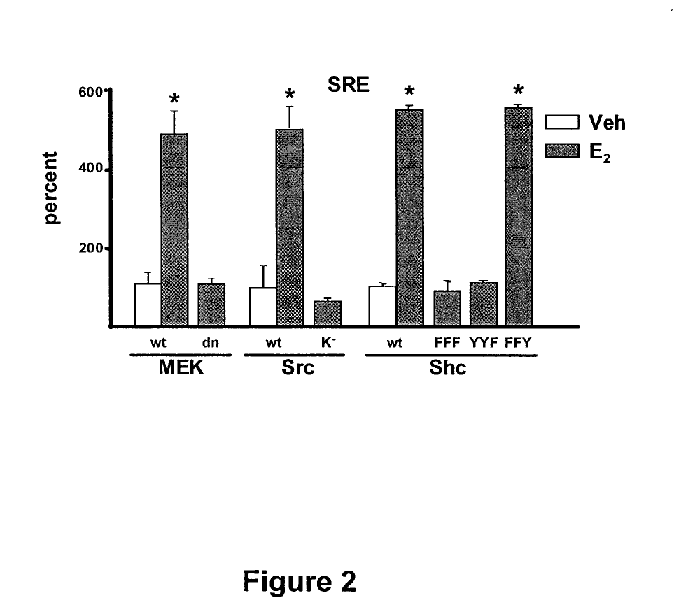

FIG. 2 illustrates that the transcriptional regulation of SRE-SEAP by estrogens requires the Src/Shc/ERK signaling pathway. HeLa cells were transfected with expression constructs encoding the full length ERα together with wt MEK or dn MEK, wt Src or a Src mutant lacking kinase activity (Src K⁻), and wt Shc or dn Shc mutants in which the primary sites of phosphorylation have been substituted by phenylalanine (Y239F/Y240F/Y317F (Shc FFF), Y317F (Shc YYF) or Y239F/Y240F (Shc FFY)). Src kinase activity and phosphorylation of Shc at tyrosine 317, the primary site of Shc phosphorylation by Src kinases, are required for stimulation of SRE activity by E₂. 100% indicates the activity in vehicle-treated cells. Bars indicate means±SD of triplicate determinations, *p<0.05 vs. vehicle by ANOVA.

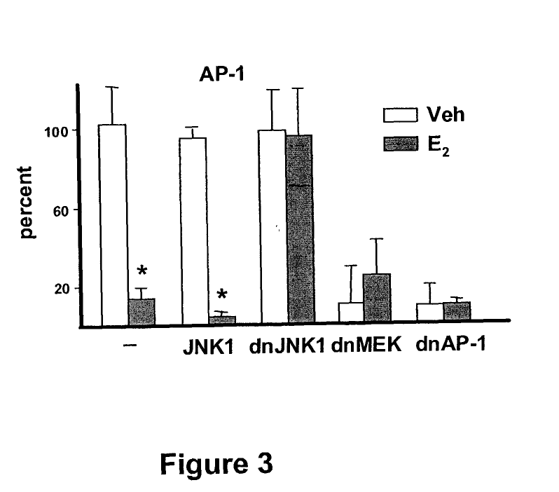

FIG. 3 illustrates that the regulation of AP-1-SEAP by estrogens requires the JNK signaling pathway. HeLa cells were transfected with expression constructs encoding the full length ERα together with wt JNK1, dn JNK1, dn MEK, or dn AP-1. 100% indicates the activity in vehicle-treated cells. Bars indicate means±SD of triplicate determinations, *p<0.05 vs. cells cultured without E₂ by ANOVA.

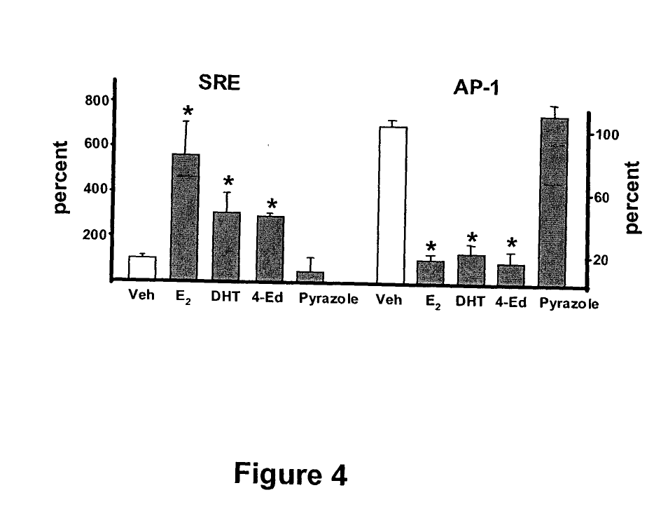

FIG. 4 illustrates that the regulation of SRE- and AP-1-mediated transcription via a sex-nonspecific, non-genotropic mechanism. HeLa cells were transiently transfected with the AR together with the SRE-SEAP or the AP-1-SEAP reporter constructs. Cells were exposed to vehicle or the indicated steroids (10⁻⁸ M) for 15 minutes. The steroid containing media were then removed, the cells were washed twice, and the cultures were continued in fresh medium without steroids. Supernatants were collected six hours later and SEAP activity was assayed. 100% indicates the activity in vehicle-treated cells. Bars indicate means±SD of triplicate determinations. *p<0.05 vs. vehicle by ANOVA.

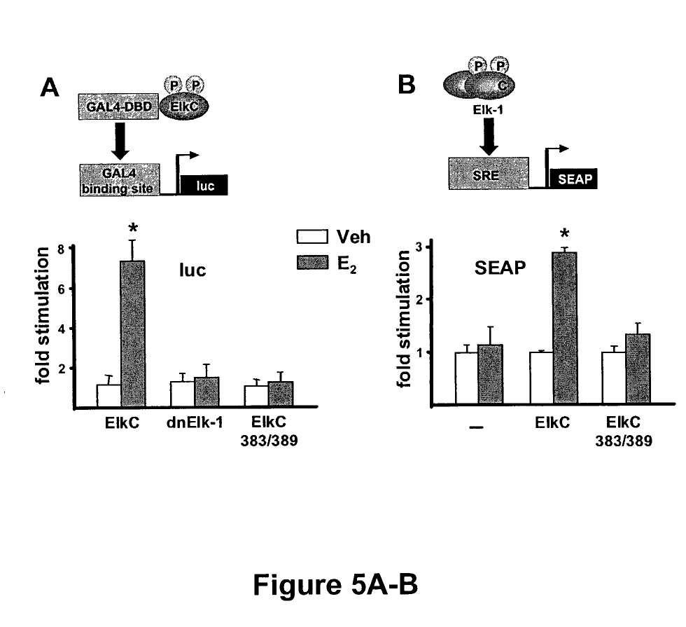

FIG. 5 illustrates that E₂-induced phosphorylation of Elk-1 is required for activation of SRE. A. HeLa cells were co-transfected with ERα and either wt or dn Elk-1 constructs. The wt control (ElkC) is a fusion protein of the C-terminal domain of Elk-1 (amino acids 307-428) containing multiple ERK phosphorylation sites (Marais et al, 1993), and the DNA binding domain of GAL4 (GAL4-DBD). ElkC activity was measured by co-transfection of a reporter plasmid in which luciferase transcription is under the control of the GALA binding site (GAL4-luc). The dn Elk-1 lacks the DNA binding domain of Elk-1. In ElkC383/389, serines 138 and 139, the targets of phosphorylation by ERKs, are substituted with alanines. E₂ induced Elk-1 activity in the presence of the ElkC construct but not in the presence of dn Elk-1 or the phosphorylation inactive ElkC383/389 mutant. B. HeLa cells were transfected with ERα, the SRE-SEAP, together with ElkC or ElkC383/389 constructs. E₂ induced potent activation of SRE-SEAP in the presence of ElkC, but not in the presence of ElkC383/389. Bars indicate means±SD of triplicate determinations, *p<0.05 vs. vehicle by ANOVA.

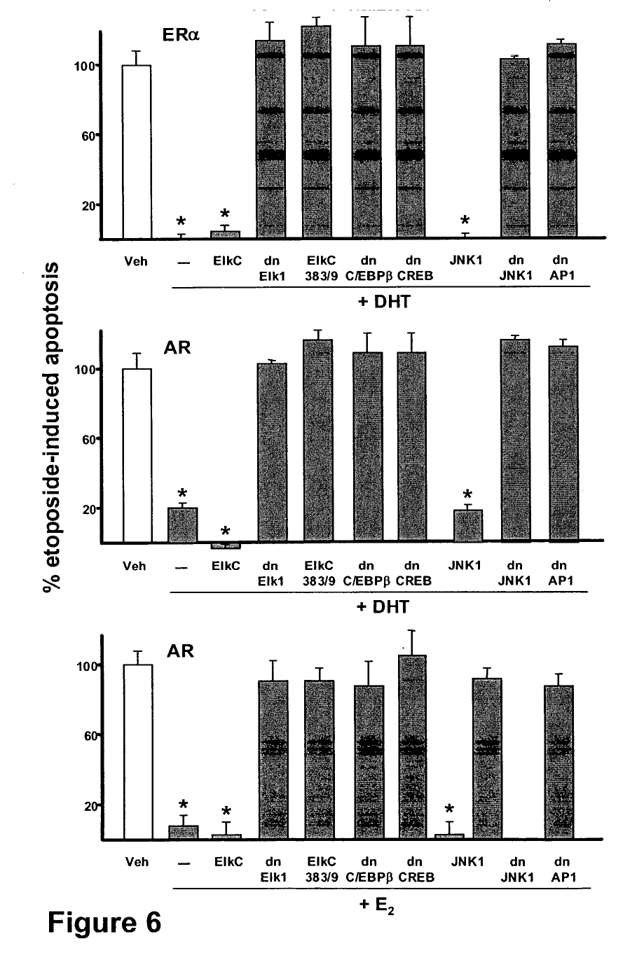

FIG. 6 illustrates that Elk-1, C/EBPβ, CREB, and JNK1/AP-1-transcriptional activity required for the anti-apoptotic effect of sex steroids is mediated by either ER or AR. HeLa cells were co-transfected with ERα (A), or AR (B and C), together with nEGFP, and wild type or dn mutants of the indicated transcription factors. Cells were then treated for 1 h with 10⁻⁸ M E₂ followed by 6 h treatment with etoposide (100 μM). Apoptosis was quantified by determining the percentage of transfected (fluorescent) cells with pyknotic nuclei. Bars indicate means±SD of triplicate determinations, *p<0.05 vs. vehicle by ANOVA. HeLa cells were co-transfected with ERα (A), or AR (B and C), together with nEGFP, and wild type or dn mutants of the indicated transcription factors. Cells were then treated for 1 h with 10⁻⁸ M E₂ followed by 6 h treatment with etoposide (100 μM). Apoptosis was quantified by determining the percentage of transfected (fluorescent) cells with pyknotic nuclei. Bars indicate means±SD of triplicate determinations, *p<0.05 vs. vehicle by ANOVA.

The development, growth, maintenance and function of reproductive tissues depend, by and large, on estrogens in females and androgens in males. However, the sex specificity of the effects of sex steroids on non-reproductive tissues is greatly relaxed. For example, estrogens are as effective in protecting against bone loss, lowering cholesterol, or slowing atherosclerosis in females as they are in males (Manolagas and Kousteni, 2001; Khosla et al, 1998; Bilezikian et al, 1998; Hodgin et al, 2001; Croniger et al, 2001; Hodgin et al, 2002; Lewis et al, 2001; Manolagas et al, 2002). Conversely, non-aromatizable androgens promote relaxation of the thoracic aorta (Komesaroff et al, 2001); and, as shown in the data provided herein, 4-estren-3α,17β-diol prevents bone loss in ovariectomized adult females.

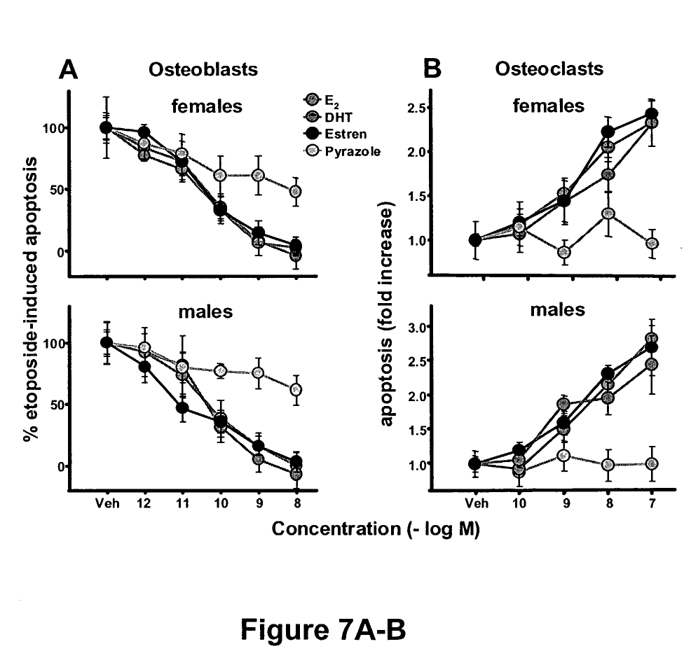

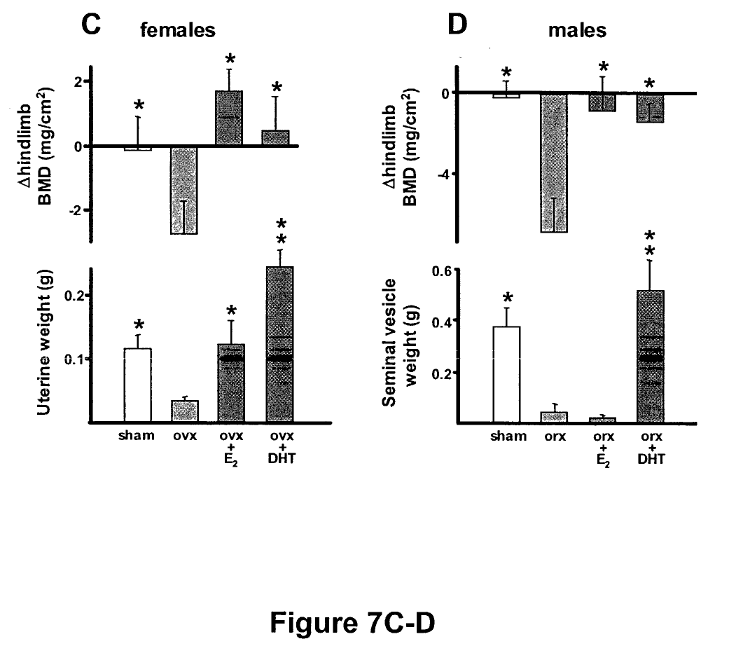

FIG. 7 demonstrates that there is an equivalence of the skeletal, but not the reproductive, actions of estrogens and androgens in female and male mice. Osteoblastic cells (A) were isolated from calvaria of neonatal female or male mice, the sex of which was determined by Southern blot analysis of liver DNA with a Y chromosome specific cDNA probe; and cultured as previously described. The ability of the indicated steroids to protect against etoposide induced apoptosis was determined as in FIG. 1D, lower panel. (B) Osteoclasts were generated in bone marrow cultures from adult female or male mice, and then treated with vehicle or the indicated concentrations of steroids for 24 hours at which point the number of cells undergoing apoptosis was determined. Representative results from 1 of 4 females and 1 of 4 males examined are shown. Each point represents the mean of triplicate determinations±SD. *p<0.05 vs. vehicle, by ANOVA. (C & D) Eight-month old Swiss Webster mice (n=8-10 per group) were sham-operated, ovariectomized (OVX) or orchidectomized (ORX). The OVX and ORX animals were then left untreated or implanted immediately with 60-day slow release pellets containing E₂ (0.025 mg) or DHT (10 mg). BMD and wet uterine or seminal vesicle weight was determined six weeks later. Bars indicate means±SD. *p<0.05 vs OVX or ORX.

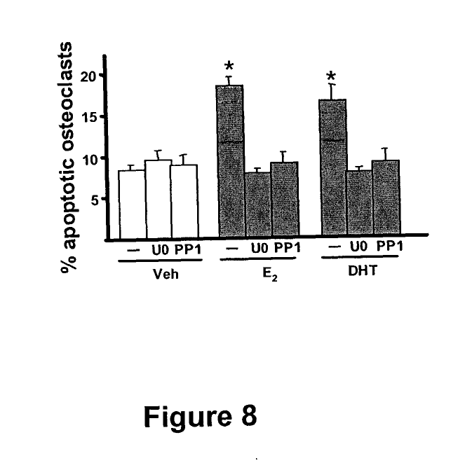

FIG. 8 illustrates the pro-apoptotic effect of sex steroids on osteoclasts requires Src/ERK signaling. Osteoclasts were pre-treated for 1 hour with U012345 or PP1, followed by addition of E₂ or DHT. After 24 hours, the percentage of apoptotic osteoclasts was determined as in FIG. 7. Bars indicate means±SD of triplicate determinations, *p<0.05 vs. vehicle by ANOVA.

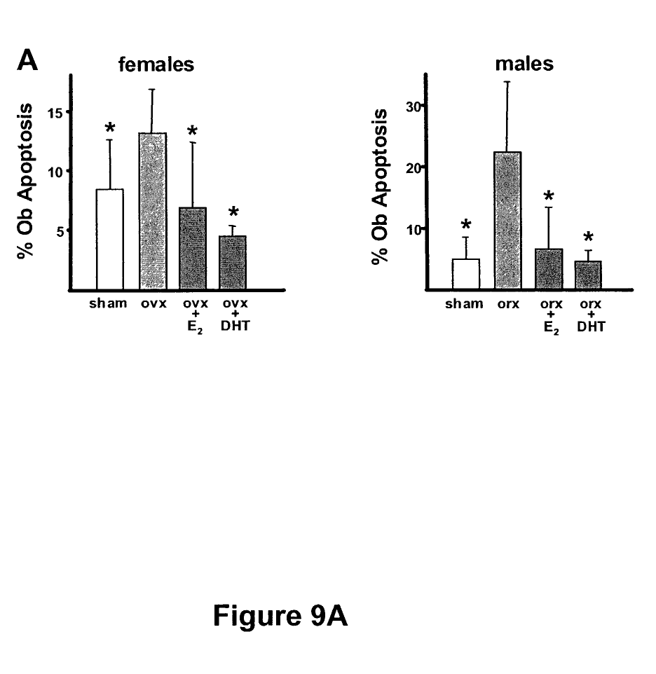

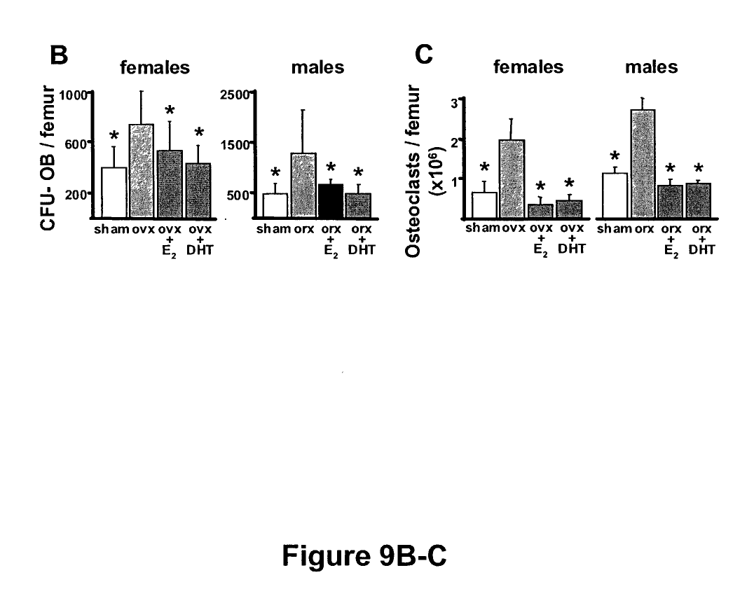

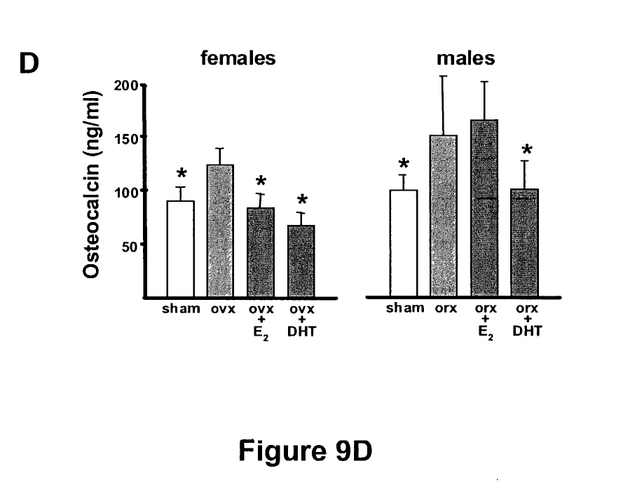

FIG. 9 illustrates the equivalence of the skeletal actions of estrogens and androgens in female and male mice. Six-month old Swiss Webster mice (n=8-10 per group) were sham-operated, ovariectomized (ovx), orchidectomized (orx), or gonadectomized and implanted immediately with 60-day slow release pellets containing E₂ (0.025 mg) or DHT (10 mg). Six weeks later, osteoblast apoptosis in histologic sections of the vertebrae (A), osteoblastogenesis and osteoclastogenesis in ex vivo bone marrow cultures (B and C), and serum osteocalcin concentration (D) were determined. Bars indicate means±SD, *p<0.05 vs OVX or ORX.

The results described above offer possible mechanistic explanations for the relaxed specificity of the effects of sex steroids on nonreproductive tissues like bone. Specifically, ER α or β or the AR can transmit signals through the Src/Shc/ERK signaling pathway with similar efficiency irrespective of whether the ligand is an estrogen or an androgen, by demonstrating the same interchangeable profile of ligand/receptor specificity in the regulation of the activity of ubiquitous transcription factors, like SRE and AP-1, and the function of proteins, e.g. Bad, downstream from kinases. Consistent with this, the data show that there is an equivalence of the effects of estrogens and androgens on the survival of bone cells in vitro and in vivo and in the protection against bone loss in males and females. In support of the contention that an interchangeable ligand/receptor interaction in bone cells from either sex is responsible for the equivalence of their effects in males and females, it has been shown that that E₂ stimulates ERK phosphorylation, prevents osteoblast apoptosis, and stimulates osteoclast apoptosis in cells from mice lacking both ERα and ERβ, or any variant of these proteins (DERKO) (Dupont et al, 2000). As in the wild type control, the effects of E₂ in the DERKO cells could be prevented by either ICI 182,780 or flutamide, (Chen et al, 2002). Furthermore, consistent with an AR-mediated effect of estrogens, DERKO mice loose bone following OVX and this can be prevented by administration of E₂ (Gentile et al, 2001).

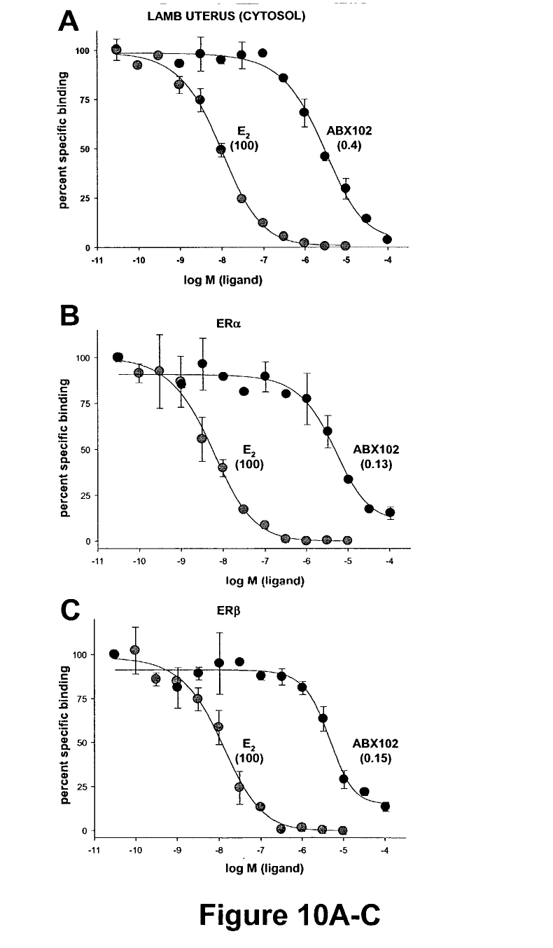

FIG. 10 illustrates the relative binding affinity (RBA) of 4-estren-3α,17β-diol and E₂ for lamb uterus cytosol, human ERα, and human ERβ. Vehicle or 11 dilutions of the indicated compounds were incubated with 10 nM ³H E₂ and 0.3-0.4 nM of the indicated protein, for 18-24 hrs at 0° C., in a buffer consisting of 50 mM Tris, pH 8.0, 10% glycerol, 0.01M mercaptoethanol and 0.3 mg/ml ovalbumin. The proteins were absorbed to hydroxylapatite (HAP) and the free ligand removed by washing (Carlson et al, 1997). Results are expressed as percent specific binding. The RBA values for 4-estren-3α,17β-diol are shown in parenthesis and are relative to E₂ in the respective protein preparation (defined as 100%). Symbols indicate means±SD of two separate experiments in which each point was determined in duplicate.

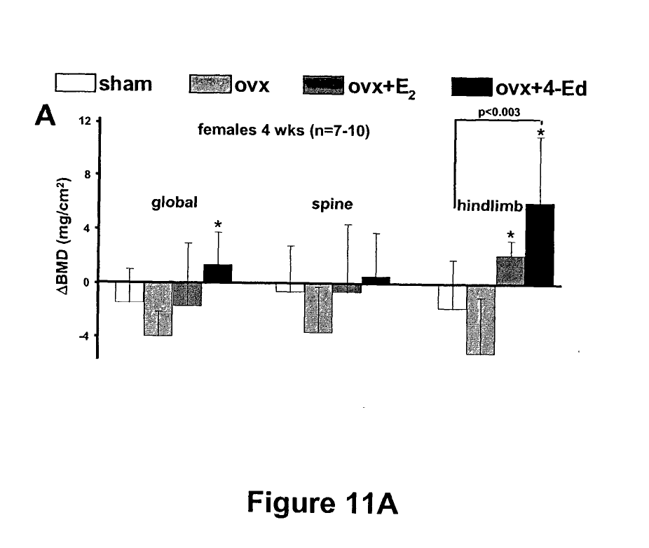

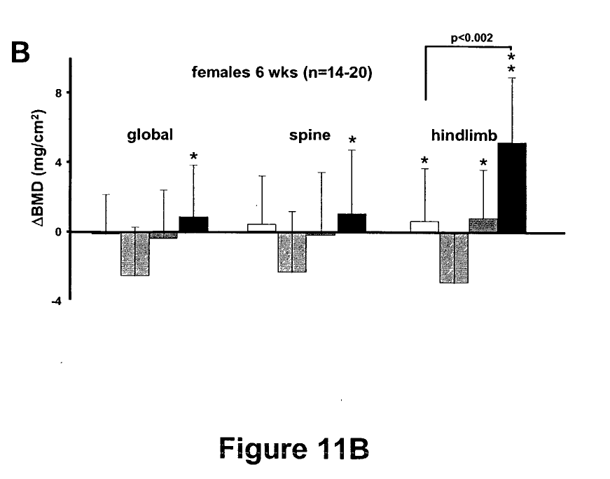

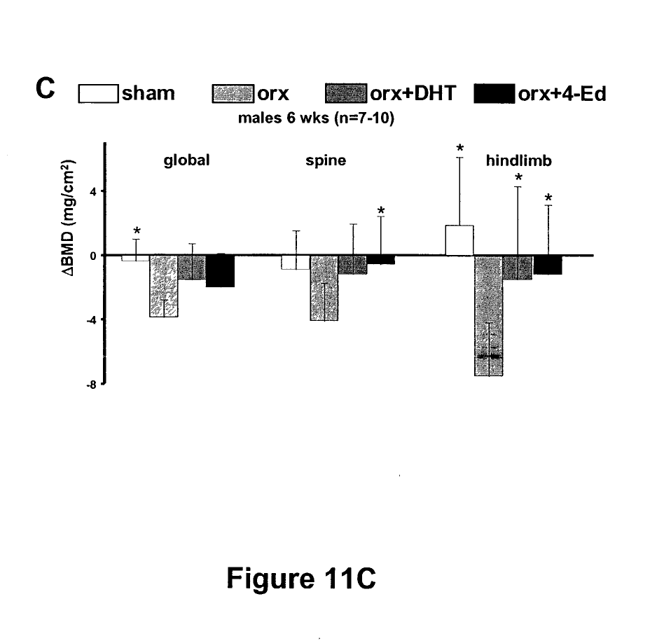

FIG. 11 demonstrates that 4-estren-3α,17β-diol increases bone density in gonadectomized mice receiving 4-estren-3α,17β-diol. In three separate experiments, 6- or 8-month old female or 6-month old male Swiss Webster mice (n=8-10 per group) were sham-operated, gonadectomized, or gonadectomized and implanted immediately with 60-day slow release pellets containing E₂ (0.025 mg), DHT (10 mg) or 4-estren-3α,17β-diol (7.6 mg). Global, spine and hindlimb BMD was determined at both 4 and 6 weeks later in the experiment with the 8 month old females, and at 6 weeks only in the experiments with the 6 month old females and males (A, B and C). The 6 week BMD data in females (B) represent pooled values from the two experiments with the 6- and the 8-month old mice.

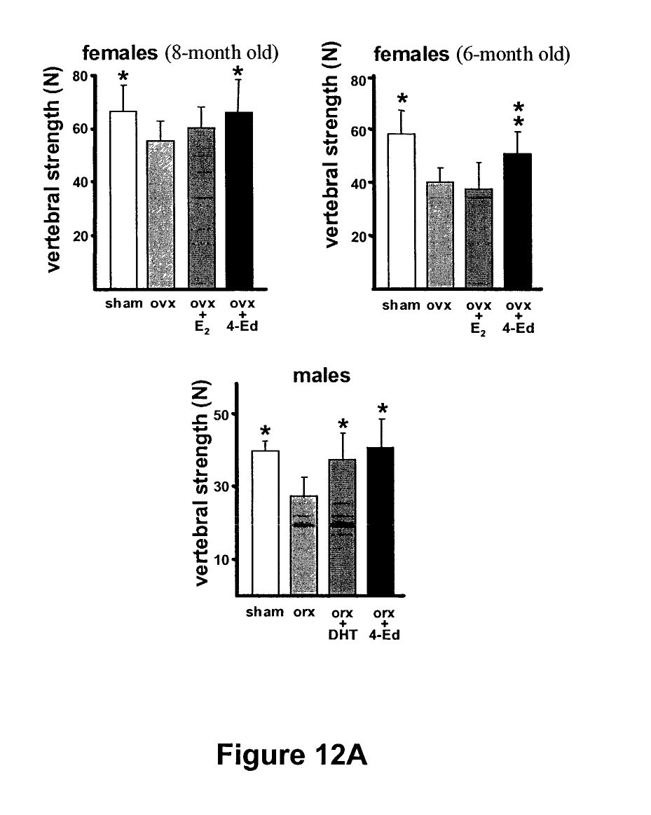

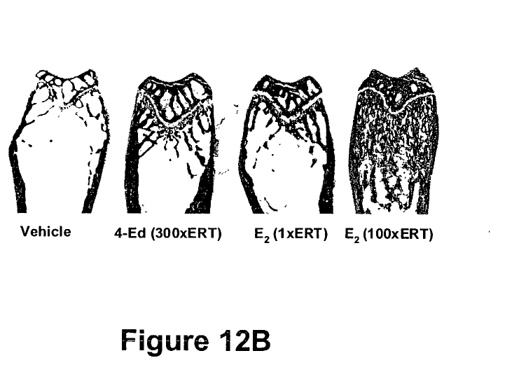

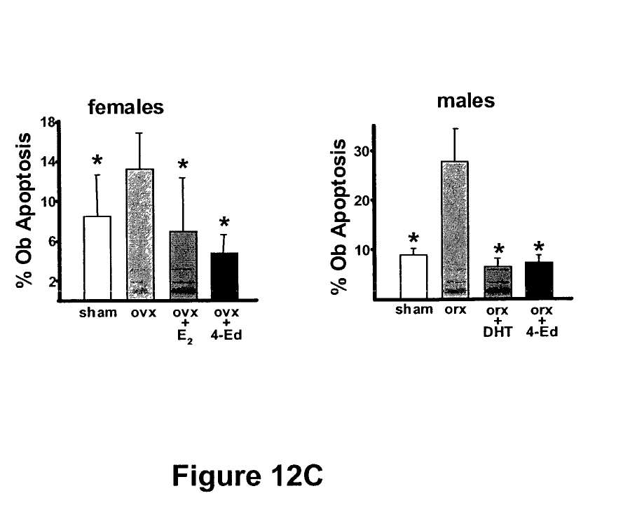

FIG. 12 demonstrates compression strength in L5 from the 6- and 8-month old female and male mice of the experiments described in FIG. 11(A). Longitudinal undecalcified sections of the distal femur are shown in (B). Note increased cortical and trabecular width in mice receiving 4-estren-3α,17β-diol, at a dose 300 times higher than an E₂ replacement dose (300× ERT) as compared to the animals receiving vehicle or E₂ at a replacement dose (1× ERT). By contrast, note the cancellous sclerosis that occurred in mice receiving E₂ at 100× ERT. Osteoblast apoptosis in sections of L1-L4 vertebrae from females (8 month old) and males (C). Bars indicate means±SD. *p<0.05 vs OVX or ORX; **p<0.05 vs OVX or ORX and vs OVX+E₂.

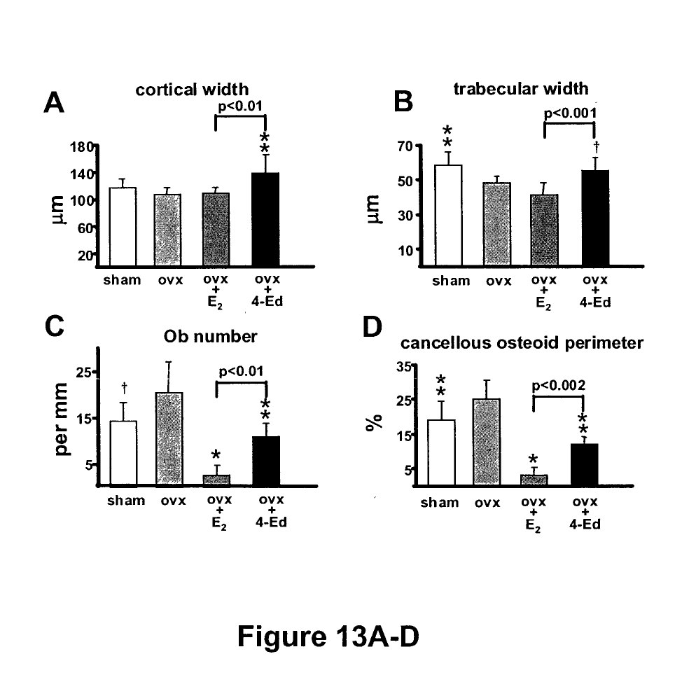

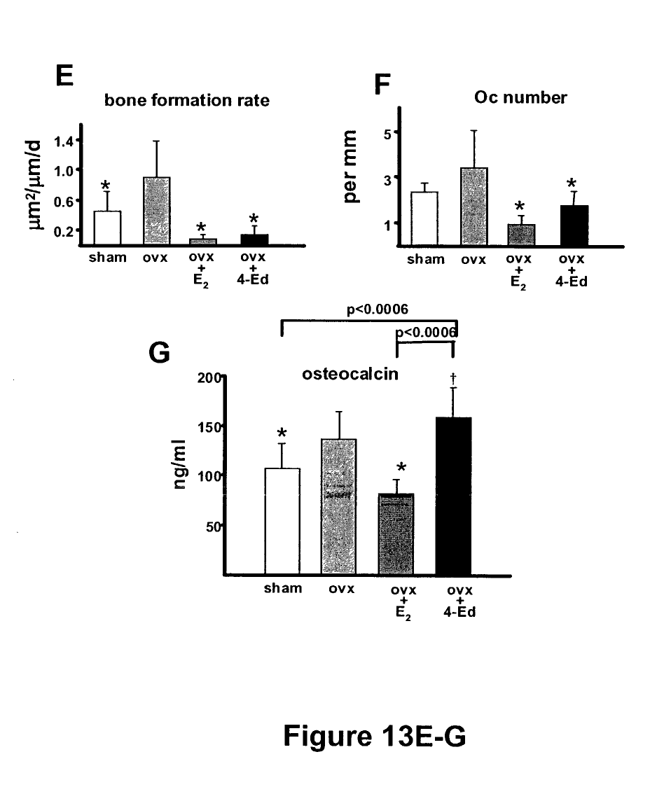

FIG. 13 demonstrates that 4-estren-3α,17β-diol increases trabecular and cortical width, osteoblast number and serum osteocalcin. Histomorphometric analysis of L1-L4 vertebrae from 6 month old females (A-F) and pooled serum osteocalcin levels from the 6 and 8 month old females (G). Bars indicate means±SD. *p<0.05 vs OVX; **p<0.05 vs OVX and vs OVX+E₂; †p<0.05 vs OVX+E₂. The ANGELS compounds described herein are useful for maintaining and/or increasing bone mass and/or strength and/or density in mammals. Mammals, preferably humans, in need of such compounds can include those suffering from such conditions as female osteoporosis (post menopausal), male osteoporosis, glucocorticoid-induced osteoporosis, immobilization and aging-related osteoporosis, idiopathic or juvenile osteoporosis, transplantation-related osteoporosis, and alveolar ridge bone loss. In addition, the compounds described herein are particularly useful for administration to subjects that are unable or unwilling to tolerate therapies that have a masculinizing or feminizing effect. For example, subjects such as breast cancer patients (especially those with bone metastasis on gonadotropin reducing hormone (GnRH) or ovariectomized), prostrate cancer patients (especially those on GnRH/castration therapy), and myeloma/lymphoma patients are frequently poor candidates for treatment with estrogen or androgens because of the risk of a recurrence of the underlying condition, e.g., cancer.

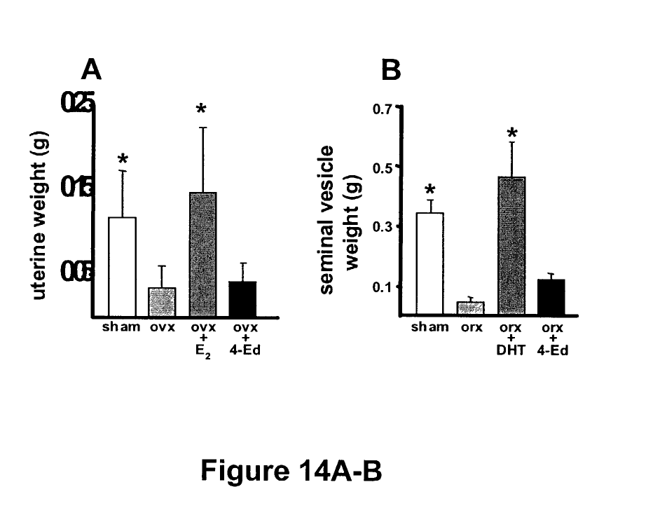

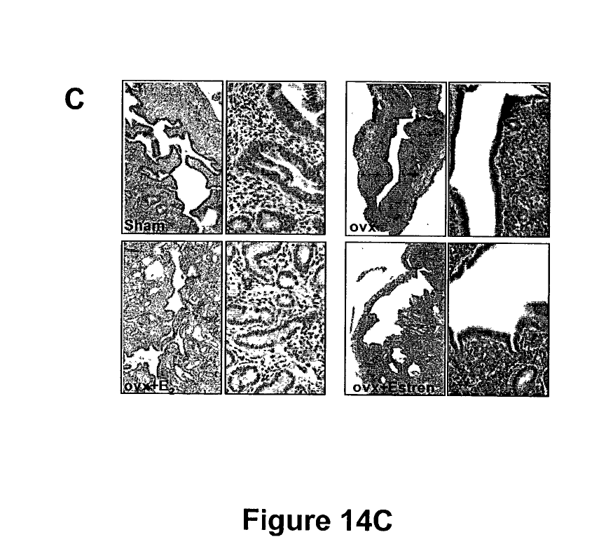

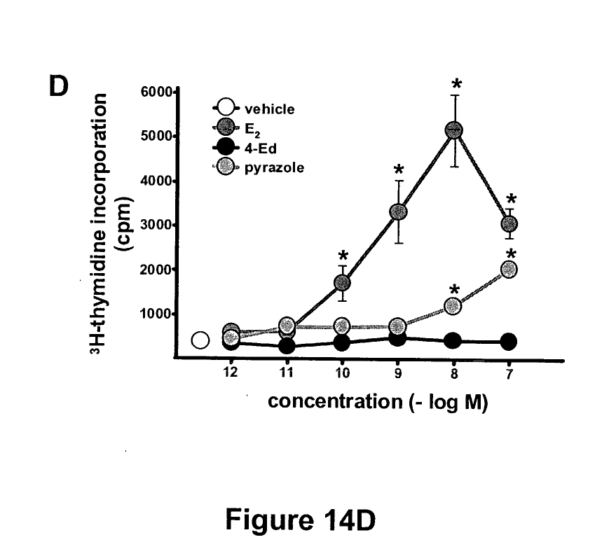

FIG. 14 demonstrates that 4-estren-3α,17β-diol lacks an effect on female and male reproductive tissues or breast cancer cells. Wet uterine (A) or seminal vesicle weight (B) of female and male mice described in FIG. 11. The lack of an effect of 4-estren-3α,17β-diol on the uterus was confirmed in a second experiment with mice ovariectomized at 8 months of age. (C) Longitudinal 0.3 μm thick paraffin sections of uteri, stained with hematoxylin, from the 6 month old mice shown in FIG. 11 (bar=100 μm). Note the following morphologic differences in the uteri of OVX- and OVX+4-estren-3α,17β-diol-treated mice as compared to uteri from sham-operated and OVX+E₂-treated mice: thin and atrophic columnar epithelium; compact stroma; decreased number of glands; decreased nucleus to cytoplasmic ratio in the columnar epithelial, endometrial stroma, and myometrial cells; and absence of mitotic activity. (D) Proliferation of MCF-7 cells was determined by ³H-thymidine uptake.

The above results show that 4-estren-3α,17β-diol faithfully reproduces the non-genotropic effects of estrogens or androgens through the ER or the AR, without affecting classical transcription, has a unique and superior effect on BMD and compressive strength than estrogens in females; and is at least as effective as DHT in males. How can elimination of the genotropic effects of sex steroids lead to a superior effect on bone? The present findings strongly suggest that bone mass depends primarily on the focal balance between formation and resorption which in turn depends on the lifespan of osteoclasts and osteoblasts, reflecting the timing of apoptosis—not on the rate of remodeling (Manolagas, 2000). Estrogens or androgens, and for that matter other agents which suppress remodeling, can cause an initial gain in bone mass by closing the temporary gap between formation and resorption created by increased remodeling. This, however, slows with time and cannot rebuild a normal skeleton. The relative increase in the number of osteoblasts and the increase in serum osteocalcin with 4-estren-3α,17β-diol suggests that this compound has the potential to cause positive focal balance between formation and resorption and continuous gain in bone mass, thereby rebuilding a normal skeleton.

The increased BMD and strength in the 4-estren-3α,17β-diol treated mice, as compared to E₂ or DHT treated animals, may result from additional mechanisms, for example an upregulation of osteoblastogenesis, or promotion of progenitors towards mature osteoblasts. This contention is consistent with the breadth of the effects of 4-estren-3α,17β-diol on the activation of several ubiquitous transcription factors utilized by factors which promote bone growth; therby indicating that ANGELS compounds must have additional biologic effects beyond the control of cell lifespan. For example, in contrast to the findings that activation of MAP kinases by an extranuclear function of the ERα suppresses c-jun activity, it has been shown that E₂-activated ERα stimulates AP-1 activity by a genotropic mechanism (Kushner et al, 2000). Hence, the response of a target cell to sex steroids may be determined by the balance between non-genotropic and genotropic actions. Consequently, at the molecular level the superior effects of 4-estren-3α,17β-diol on bone could very well be due to the removal of a counterregulatory effect on AP-1. According to this hypothetical scenario, a greater suppression of c-jun could lead to decreased transcription of the Wnt antagonist Dickkopf thereby unleashing Wnt signaling—a potent bone anabolic stimulus (Boyden et al, 2002; Grotewold and Rüther, 2002).

The evidence described herein demonstrates that ANGELS compounds can selectively activate kinase originated signaling cascades, via a non-genotropic action of the ER or the AR, but lack the ability to induce the classical transcriptional activity of these receptors, thus eliciting unique biologic outcomes: they dissociate the skeletal from the reproductive effects of sex steroids. In agreement with this evidence, inactivation of both the genotropic and non-genotropic function of the glucocorticoid receptor causes lethality in mutant mice, whereas elimination of the transcriptional activity of this receptor does not—literally a difference between life and death (Reichardt et al, 1998). Based on this understanding, it is believed that mechanism-specific ligands of the ERs or the AR (as opposed to tissue-specific ligands (SERMs) or classic estrogens or androgens), and perhaps mechanism-specific ligands of other nuclear receptors, represent a novel class of pharmacotherapeutics.

Sex steroid replacement, during late postreproductive life, is a therapy whose benefits derive primarily from the actions of sex steroids on nonreproductive tissues, whereas its side effects result from actions on reproductive ones. This truism is highlighted by a massive effort to develop selective estrogen receptor modulators (SERMS) that act as estrogen agonists on non-reproductive tissues like bone, but as antagonists in reproductive tissues, i.e. uterus and breast. Because of the superior and gender-neutral effects of 4-estren-3α,17β-diol on bone and its lack of an effect on reproductive tissues, mechanism specific ligands, such as 4-estren-3α,17β-diol, are an advantageous modality for sex steroid replacement therapy, compared to estrogens or SERMS (Doran et al, 2001; Ott et al, 2002). Growing concern with the efficacy and safety of existing hormone replacement therapies (Santoro et al, 1999; Herrington et al, 2000; Manson and Martin, 2001; Mosca et al, 2001) makes these discoveries timely, as they provide for a bone anabolic, sex neutral hormone replacement therapy.

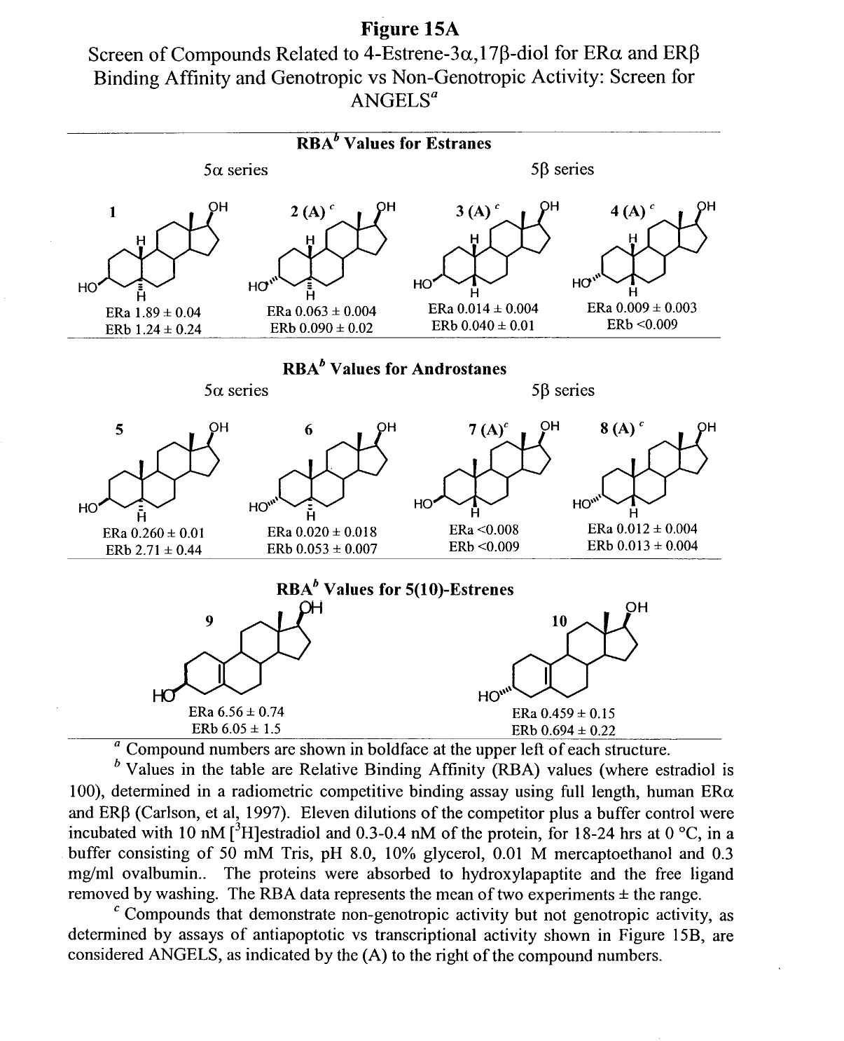

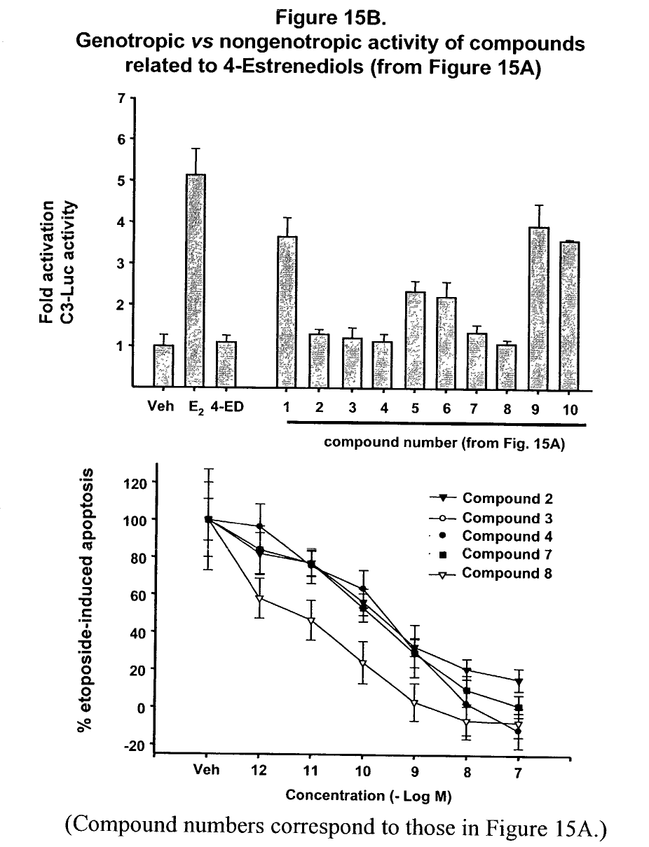

FIG. 15 illustrates the results of screening for genotropic vs nongenotropic activity of compounds related to 4-estren-3α,17β-diol (from scheme 1A), and is discussed in greater detail below.

### EXAMPLES

### Synthesis of Bone Anabolic Compounds

Synthesis of Estrenediols and Estranediols

The synthesis of various estrenediols, estranediols, androstenediols, and androstanediols that are epimeric at positions 3, 5, and 17 is illustrated in Scheme 1C. Most of these compounds are known and can be prepared by literature methods.

Starting materials testosterone and 19-nortesterone are commercially available (e.g., from Steraloids, Inc., Newport, R.I.). The more common 17β epimeric alcohols are inverted by the Mitzunobu method (Smith and March, 2001) to give the 17α epimeric alcohols.

Borohydride reduction gives the various 4-estrenediols and 4-androstenediols. Typically, epimeric mixtures of 3α and 3β alcohols are obtained, but these can be separated by chromatography or crystallization. Sometimes, bulky hydride reagents (such as sodium tri-t-butoxy aluminum hydride or lithium diethylborohydride) give improved selectivity in the formation of either the 3α or 3β epimeric alcohols.

Dissolving metal reduction of the A-ring enones, followed by borohydride reduction gives the various estranediols and androstanediols.

Formation of the 3-dienyl-17-diacetate, followed by borohydride reduction and alkaline hydrolysis gives the various 5(6)-estrenediols and 5(6)-androstenediols.

To produce the various 5(10)-estrenediols, estradiol methyl ether is inverted by the Mitzunobu method, and each epimer is subjected to Birch reduction, followed by treatment with a weak acid, such as oxalic acid (Smith and March, 2001). Borohydride reduction gives the various 5(10)-estrenediols. Where epimeric alcohols are produced by hydride reduction, the stereoisomers are separated.

Synthesis of Estrenediols and Estranediols with Ring Substitutions

The synthesis of various estrenediols bearing affinity-enhancing substituents at 17α (System A), 7α (System B), and 11β (System C) is illustrated in Scheme 2B. To prepare these three classes of substituted estrenediols, 3-methyl ether derivatives of estradiol or the known 7α or 11β-substituted estrogens (French et al., 1993b; Pomper et al., 1990) are subjected to Birch reduction followed by treatment with strong acid (Smith and March, 2001) to give the corresponding conjugated enones.

To prepare the 17α-substituted estrene (System A), the 3-ketone is first selectively protected by formation of the 3-dienyl ether (Fried and Edwards, 1972) so that the 17-alcohol can be oxidized to the ketone. Lithium trimethylsilyl acetylide (or other suitable Grignard or lithium reagents) can then be added selectively to give the 17β alcohol. The dienyl ether is then hydrolyzed with weak acid, and the 3-ketone is reduced with sodium borohydride to give the desired 17α-substituted estrene.

In the case of the 7α-substituted estrenediols (System B) or the 11β-substituted estrenediols (System C), the desired substituent is already present in the starting estrogen derivative, having been introduced by the methods noted in the references given. The desired estrenediols are obtained simply by reducing the 3-ketone sodium borohydride.

Analogs having various combinations of the 7α, 11β, and 17α substitutions can be made, as can analogs having different substituents than are illustrated here. Where epimeric alcohols are produced by hydride reduction, the stereoisomers are separated. The stereochemistry at any of the secondary alcohol positions can also be inverted by the Mitzunobu sequence, in which the alcohol is treated with triphenylphosphine, diisopropyl azodicarboxylate, and sodium benzoate. The epimeric benzoate that is obtained is then hydrolyzed to the epimeric alcohol by treatment with K₂CO₃ in refluxing ethanol or with KOH in aqueous dioxane.

Synthesis of Androstenediols and Androstanediols

The synthesis of the various androstenediols and androstanediols from testosterone and dihydrotestosterone is illustrated in Scheme 1C and follows methods that parallel those shown for the corresponding estrenediols and estranediols.

Synthesis of Androstenediols and Androstanediols with Ring Substitutions

Many androstenediols and androstanediols having substituents at the 17α position are known and some are available commercially.

Those androstenediols and androstanediols with substituents at the 7α position can be prepared by a copper-catalyzed 1,6-conjugate addition of a suitable Grignard or organolithium reagent on 6-dehydrotestosterone 17-t-butyl-dimethylsilyl ether. After cleaving the 17 protecting group by treatment with tetrabutylammonium fluoride, the 7α-substituted testosterone can be converted into various 7α-substituted androstenediols and androstanediols by the same methods used to prepare the corresponding estrenediols or estranediols.

Androstenediols and androstanediols with substitutents at the 11β position can be prepared from the known 1,4-androstadien-3,11,17-trione. Treatment with ethylene glycol and toluenesulfonic acid effects selective ketalization of the 17-ketone. Careful treatment of this dione with 1 equiv of a vinyl Grignard reagent will effect selective addition to the more reactive C-11 ketone. The resulting 11 allylic alcohol can be selectively dehydroxylated by treatment with triethylsilane and trifluoroacetic acid, giving selectively the 11β-vinyl substituted product. The ketal is then cleaved, and mild catalytic hydrogenation results in reduction of the double bonds at C-1 and on the 11β substituent. Borohydride reduction gives the 11β-substituted androstenediols. More vigorous hydrogenation results in reduction, as well, of the double bond at C-4, furnishing, after borohydride reduction, the 11β-substituted androstanediols.

Synthesis of Norsteroids, Homosteroids, Secosteroids, and Cyclosteroids that are Derived from Estrenediols and Estranediols

Examples of the synthesis of nor-, homo-, seco-, and cyclo-steroids that are derived or patterned after estrenediols or estranediols are given in Scheme 3C.

An example of an A-nor-estrane (System A) is prepared by a standard ring contraction reaction, starting from 19-nortestosterone. A 2-diazo function is introduced by treating the ketone with ethyl formate and sodium hydride, to generate the 2-formyl ketone, followed by tosylazide, which effects a diazotransfer reaction and a spontaneous deformylation squence (Larock, 1989; Paquette, 1995). Curtius rearrangement (Smith and March, 2001), which occurs by photolysis of the diazoketone (sunlamp irradiation through Pyrex), gives the ring-contracted acid. Treatment of this acid with lead tetraacetate (Paquette, 1995) results in an oxidative decarboxylation reaction, giving the desired ring-contracted nor-steroid alcohol.

As an example of the synthesis of a homo-steroid related to an estrane (System B), 19-nortestosterone is put through a ring-expansion sequence: Wittig methylenation of the C-3 ketone is followed by dihydroxylation with osmium tetroxide (Paquette, 1995), giving the glycol. Selective reaction of the primary alcohol function with toluenesulfonyl chloride and pyridine to give the monotosylate and then treatment with sodium methoxide, effects a regioselective Pinacol rearrangement (Smith and March, 2001), yielding the ring-expanded ketone. Borohydride reduction gives the desired ring-expanded alcohol (homo-steroid).

The example given for the synthesis of a seco-steroid related to estrene (System C) starts with estrone 3-methyl ether. This material is thermolyzed with strong alkali, in a well-precedented reaction, to give the Doisynolic acid (Chi et al., 1995; Scribner et al., 1997). Birch reduction of the A-ring phenyl methyl ether, followed by strong acid treatment during the workup gives the conjugated enone alcohol. Simple borohydride reduction then gives the desired D-ring seco-steroid related to estrene.

The synthesis of many cyclosteroids of the estrene type (System D) can be made quite simply by the addition of p-methoxyphenyl lithium to cyclic ketones (or monoketals of cyclic diketals). The benzylic hydroxyl group that is produced can either be removed by a silane-mediated dehydroxylation process (Larock, 1989), or replaced with an alkyl group by treatment with a trialkyl aluminum and a strong Lewis acid such as aluminum chloride. Birch reduction of the phenyl methyl ether and acid treatment as before, followed by borohydride reduction, gives the desired cyclo-steroid estrene analog. Where epimeric alcohols are produced by hydride reduction, the stereoisomers are separated.

The structures for estrenediol analogs that correspond to certain non-steroidal steroid mimics and may be considered related to seco steroids are shown in System E (R¹, R², and R³ in these structures are C₁-C₅ alkyl groups). These are analogs derived from the known non-steroidal estrogens hexestrol and benzestrol. They may be prepared from hexestrol or benzestrol by certain simple reactions—the two six-membered rings in hexestrol and benzestrol are phenolic, and either one or both of these phenols can be converted to a phenyl group or to a cyclohexenol or cyclohexanol. To make the conversion to a phenyl group, either one or both of the phenolic hydroxyl groups are converted to the corresponding methanesulfonate ester and then this compound is subject to catalytic hydrogenolysis by exposure to hydrogen over a palladium catalyst on carbon support. To convert the phenol to the other two ring types (cyclohexenol or cyclohexanol), the following sequence is used: Either one or both of the phenols are converted to the methyl ether using methyl iodide and potassium carbonate in ethanol. Birch reduction (lithium metal in liquid ammonia and ethanol), followed by workup with strong acid, will convert the phenyl methyl ether ring to a conjugated cyclohexenone; a ring with a free phenol will not be reduced under these conditions. Borohydride reduction of the cyclohexenone ring then gives the corresponding cyclohexenol. The cyclohexanol ring can be obtained by hydrogenation of the cyclohexenol ring with hydrogen over a palladium catalyst on a carbon support.

Many variations of these routes that lead to other nor-steroids and homo-steroids where different rings (such as the B-, C- or D-rings) are reduced or enlarged, seco-steroids in which different rings (such as the B- or C-rings) are cleaved, cyclosteroids having different ring sizes and other substituents, and analogs of non-steroidal estrene-like compounds can easily be envisioned by those skilled in the art of organic synthesis. In addition, catalytic hydrogenation can be used to prepare the corresponding estrane analogs of the nor-, homo-, seco- and cyclo-steroids. In certain syntheses, the use of protecting groups may be required to avoid functional group interactions.

Synthesis of Heterocyclic-Core Estrene Analogs

Examples of the synthesis of four different heterocyclic core estrene analogs are given in Scheme 4C. Details for the synthesis of these four systems are given below.

The pyrimidine estrene analog (System A) is constructed by condensation of an amidine, readily prepared from a simple nitrile, with a 1,3-dione system. 1,3-Cyclohexadiene (Aldrich) is converted to the monoepoxide by treatment with 1 equiv of m-chloroperoxybenzoic acid (m-CPBA) in dichloromethane for 1 h at RT. The monoepoxide is treated with 1 equiv of diethylaluminum cyanide in dichloromethane at −78 to 25° C. over 3 h to effect an S_{N}2′ addition which generates the cyano-cyclohexenol. Treatment of this nitrile with a 10-fold excess of lithium hexamethylsilyl amide in THF, followed by a 30-fold excess of TMS-Cl, produces the corresponding per-silylated amidine, which is the first component needed for the condensation. The 1,3-diketone component is prepared from a suitable 1,3-diketone, such as 2,4-pentanedione (R′, R″═Me) (Aldrich). The corresponding enolate, generated using 1 equiv of NaH in THF, is treated with 1 equiv of a aldehyde, such as propanal, isobutyraldehyde, or benzaldehyde, to form the aldol addition product. Other 1,3-diketone precurors are commercially available (Aldrich) or can be produced by Claisen condensation between and ester and an ester enolate, derived either from the same ester (symmetrical) or two different esters (unsymmetrical), followed by alkaline hydrolysis (5 N KOH in MeOH for 6 h at RT). The β-ketoacid can be decarboxylated to generate the 1,3-diketone. The pyrimidine is then generated by treatment of equimolar amounts of the persilylated amidine and the 1,3-diketone with 0.3 equiv of ammonium chloride in THF at reflux for 10 h.

The thiophene analog (System B) is constructed from a 3,4-disubstituted thiophene by a double metallation-addition sequence. 3,4-Dialkyl-thiophenes are either commercially available or can be prepared by a sequence that begins with a nitrile coupling reaction. Either a single nitrile (symmetrical) or two different nitrites (unsymmetrical) are converted to their corresponding anions (2 equiv NaH, THF, 35° C., 1 h) and then treated at 0° C. with 0.5 equiv of I₂. With the unsymmetrical coupling, the mixed bis-nitrile is separated from the two symmetrical bis-nitriles. The bis-nitrile is reduced to the bis-aldehyde by treatment with a 6-fold excess of diisobutylaluminum hydride in toluene at −78° C. for 6 hours. Exposure of the bis-aldehyde to an excess of H₂S and anhydrous HCl in dichloromethane at RT for 6 h produces the corresponding 3,4-disubstituted thiophene. The substituents at positions 2 and 5 are introduced by two cycles of a metalation-addition sequence. Treatment of the disubstituted thiophene with 1.5 equiv of n-butyllithium in THF at −30° C. for 1 h, conversion to the corresponding cuprate by the addition of one equiv of cuprous bromide dimethylsulfide complex, followed by treatment with an excess of the monoepoxide of 1,3-cyclohexadiene (see System A, above), produces the trisubstituted thiophene. A second cycle of this process, using a suitable aldehyde in place of the epoxide and without converting the thiophenyl lithium to the cuprate, produces the desired tetrasubstituted thiophene.

The pyrrole estrene analog (System C) is prepared by the condensation of a suitable hydroxycyclohexyl hydrazine with a 1,3-dione. The hydrazine component is prepared by reacting equimolar amounts of the hydrazine with the 1,3-cyclohexadiene monoepoxide (see System A) in ethanol at 50° C. for 1 h. The 1,3-diketone component is prepared as follows: A suitable 1,3-diketone, prepared by methods outlined in System A, which may also be substituted at the α position with an alkyl group by standard enolate alkylation methods (treatment with 1 equiv of NaH in THF, followed by an excess of alkylating agent), is converted to the dianion (treatment treatment with 1 equiv of NaH in THF a RT, followed by 1 equiv of BuLi at −20° C.) and then treated with 1 equiv of MoOPH (molybdenum pentoxide pyridine hexamethylphosphoric triamide) for 1 hr at −20 to 25° C. to give the hydroxy-1,3-dione. The hydrazine component and the 1,3-dione component are then mixed together and warmed in ethanol (25 to 60° C.) for 12 h to produce the pyrazole.

The pyridine estrene analog (System D) is prepared by the reaction of 4-hydroxy-piperidine (Aldrich) with a 2-chloropyridine precursor. The chloropyridine is prepared by the following sequence: a 1,4-diketone, which is commercially available or can be prepared by reaction of a methyl ketone enolate with 0.5 equiv of iodine, is treated with an excess of sodium cyanide and ammonium chloride (propanol at reflux, 12 h) to prepare the pyridone intermediate, which is converted to the required chloropyridine by treatment with phosphorous oxychloride in 1,2-dichloroethane (reflux, 2 h). Treatment of the chloropyridine with 4-hydroxypiperidine (1,2-dimethoxyethane, reflux, 1 h) gives the aminopyridine adduct. This hydroxyl group on compound is protected as the benzoate (excess benzoyl chloride and toluene, 2 N aqueous Na₂CO₃) and then it is brominated with 1.5 equiv of Br₂ and 0.2 equiv of ZnBr₂. The protected bromopyridine is converted to the organozinc reagent (Rieke Zn, THF, 0-25° C.) and then treated with an excess of aldehyde. This adduct is then saponified (2 N NaOH in dioxane-water 1:1) to give the desired pyridine product.

The synthesis of other estrene analogs in which the core of the ligand is replaced by a heterocyclic system, such as the pyrazine and pyridazine and related systems shown in Scheme 4A, can also be envisioned by those skilled in the art of organic synthesis using methods related to those described above. In addition, catalytic hydrogenation can be used to prepare the corresponding estrane analogs of the heterocyclic core systems.

Synthesis of Heteroacyclic-Core Estrene Analogs

Examples of the synthesis of three different heteroacyclic-core estrene analogs are given in Scheme 4D. The three heteroacyclic-core estrene analogs shown are prepared by simple amide or urea forming processes.

The trifluoromethyl-substituted amide (System A) was prepared from three components. The trifluoromethyl ketone component was prepared from a methoxyethoxymethyl (MEM) ether protected 4-hydroxycyclohexane carboxaldehyde by the addition of trifluoromethyl anion (generated in situ by the action of tetrabutylammonium fluoride on trifluoromethyl trimethylsilane). The resulting trifluoromethyl carbinol was oxidized using the Dess-Martin periodinane to give, after MEM ether cleavage, the desired trifluoromethyl ketone. The cyclohexane carboxylic acid was prepared from a common methoxycarbonyl cyclohexenone (prepared by a Robinson annulation sequqence), which was hydrogenated to give the cyclohexanone, and then reduced selectively with NaBH₄ to the cyclohexanol. Hydrolysis gave the desired acid. The desired amide was assembled by first performing a reductive amination sequence between the trifluoromethyl ketone and cyclohexyl amine (Aldrich) in which the corresponding imine, generated as shown, was reduced by sodium cyanoborohydride. The resulting secondary amine was then coupled with the cyclohexane carboxylic acid, prepared above, using a carbidiimide reagent (dicyclohexylcarbodiimide, DCC), to give the desired amide.

To prepare the related trifluoromethyl-substituted urea compound (System B), the secondary amine, prepared as described above, was first activated with carbonyl diimidazole and then treated with a 4-hydroxypiperidine to give the desired urea.

The final amide system (System C) was prepared by DCC coupling of a complex aniline (prepared by reductive amination of a ketone with aniline, as done in System A) and the cyclohexane carboxylic acid, whose preparation is also described above in System A.

Many variations of these routes that lead to other heteroacyclic-core estrene analogs can easily be envisioned by those skilled in the art of organic synthesis. Catalytic hydrogenation can also be used to prepare the corresponding heteroacyclic-core estrane analogs. In certain syntheses, the use of protecting groups may be required to avoid functional group interactions.

Synthesis of Estratrienols with Carbocyclic Cores

Examples of the synthesis of various estratrienols with carbocyclic cores are given in Scheme 5C.

Various A-ring phenol isomers of estradiol are known, and these can be converted into the corresponding carbocyclic estratrienols (System A) by dehydration of the 17-hydroxyl group, followed by hydrogenation of the 16-dehydro-steroid.

Seco-estratrienols with carbocyclic cores (System B) can be prepared by ring fragmentations, using the same methods that were illustrated earlier in Scheme 3C, System C, and related methods. The example here starts from the commercially available 6-dehydroestradiol. The B-ring is cleaved by ozonolysis (being careful not to overoxidize so as to affect the A-ring phenol), followed by mild reductive workup effected by treating the ozonide with dimethylsulfide. The resulting dialdehyde is converted into the dimethyl analog by a double Wolff-Kishner reduction using hydrazine and concentrated KOH solution or by a Cagliotti reaction involving conversion of the aldehydes to the tosylhydrazones and then reducing these with sodium cyanoborohydride. The 17-hydroxyl group is removed by dehydration and catalytic hydrogenation, as above in the synthesis in System A.

Ring expanded (nor-estratrienols, System C) and ring-contracted (homo-estratrienols; System C) can be prepared by the same methods outlined in Scheme 3C (System A and System B, respectively).

Synthesis of Estratrienols with Heterocyclic and Heteroacyclic Cores

Examples of the synthesis of various estratrienols with heterocyclic or heteroacyclic cores are given Scheme 5D.

The heterocyclic estratrienols can be prepared using standard heterocycle synthesis methods (Gilchrist, 1992; Gupta et al., 1999; Joule et al., 1995; Eicher and Hauptmann, 1995). For the example of the pyrimidine-core estratrienol (System A), the method outlined previously in Scheme 4C (System A) can be used. Specifically, an appropriate amidine (or a persilylated amidine) is condensed with an appropriate 1,3-diketone.

The preparation of a typical thiophene-core estratrienol (System B) begins with the 3,4-disubstituted thiophene (see Scheme 4C, System B). This thiophene is metalated with 1 equiv of butyllithium, converted to the zinc chloride derivative (1 equiv of anhydrous ZnCl₂), and then coupled with p-iodophenol in a palladium-catalyzed Negishi reaction. The trisubstituted thiophene produce is then further substituted by electrophilic addition by an aldehyde, catalyzed by a Lewis acid such as SnCl₄, to give the final thiophene-core estratrienol.

An example of a heteroacyclic-core estratrienediol (System C) is prepared by routes similar to those shown in Scheme 4D. An amine (prepared as in Scheme 4D, System A) is condensed with para-hydroxy benzoic acid to give the amide shown. Many variations of these routes that lead to various other heterocyclic-core and heteroacyclic-core estratrienol analogs (in particular those that are the estratrieneol analogs of the heterocyclic and heteroacyclic estrenes shown in Schemes 4A, 4C and 4D) can easily be envisioned by those skilled in the art of organic synthesis.

Methods of Enhancing Bone Mass, Density and/or Strength by Administering Bone Anabolic Compounds

Preferred ANGELS compounds are bone anabolic compounds. Activation of the ERKs and JNK kinases leads to serum response element (SRE) and AP-1 dependent transcription, respectively (Hill and Treisman, 1995; Treisman, 1996). Based on this evidence and the earlier finding that 17β-estradiol (E₂), dyhydrotestosterone (DHT), as well as an unidentified estren, but not a pyrazole (Mortensen et al, 2001; Sun et al, 1999), activate ERKs in a non-genotropic manner, the inventors searched for the effects of these ligands on SRE-, or AP-1-dependent transcription downstream from cytosolic kinases. Exposure to E₂ for as little as five minutes was sufficient to stimulate SRE- and downregulate AP-1-dependent transcriptional activity in HeLa cells (FIG. 1A). Moreover, and exactly as shown before for the anti-apoptotic effect of E₂ on osteoblasts and osteocytes, E₂-induced SRE activation was blocked by a dn MEK, the kinase responsible for ERK phosphorylation. Similarly, the effect of E₂ on SRE was abrogated by dn Src or Sch mutants (FIG. 2). The downregulation of AP-1-SEAP activity by E₂ was abolished by a dn JNK1 mutant (FIG. 1A). A dn MEK or a dn AP-1 were ineffective in this respect, but they did decrease basal AP-1-SEAP activity (FIG. 3). Collectively, these results establish that the regulation of SRE- and AP-1-activity by E₂ results from the activation of the Src/Shc/ERK signaling and JNK cascades, respectively. Identical effects to those obtained in HeLa cells transfected with the full length ERα were demonstrated in cells transfected with a mutant consisting only of the ligand binding domain (E) (FIG. 1A). In full agreement with the earlier observations on the activation of the Src/Shc/ERK signaling pathway and anti-apoptosis, targeting the E domain to the plasma membrane (E-Mem), but not to the nucleus (E-Nuc), preserved the hormonal effects on both SRE and AP-1, demonstrating that direct receptor/DNA interaction is dispensable for the regulation of SRE- and AP-1 activity by E₂; and that this effect requires extranuclear localization of the receptor protein.

Stimulation of SRE and downregulation of AP-1 was also demonstrated in ERα transfected HeLa cells treated with DHT or 4-estren-3α,17β-diol, but not the pyrazole, indicating that ER- dependent SRE and AP-1 regulation occurs via a nongenotropic mechanism of receptor action (FIG. 1B). Importantly, HeLa cells transfected with an empty vector, instead of the ER did not exhibit the effects of E₂, establishing that these phenomena were ER-dependent (data not shown). Albeit, identical results to those shown with HeLa cells transfected with ERα were obtained using HeLa cells transfected with the AR (FIG. 4), indicating that ER- or AR-dependent SRE and AP-1 regulation results from an interchangeable sex steroid/receptor interaction, i.e. E₂ can act through AR and DHT through the ER.

Elk-1, C/EBPβ and CREB are transcription factors that can all be activated by ERKs (Cruzalegui et al, 1999; Buck et al, 1999; Bonni et al, 1999). It was investigated whether transcription in general and these factors in particular, were involved in the activation of SRE and the anti-apoptotic effects of estrogens. The RNA synthesis inhibitor actinomycin D or the protein synthesis inhibitor cyclohexamide, at doses at which they inhibited ³H-uridine or ³H-leucine incorporation, respectively without affecting cell viability, abrogated the protective effect of E₂ on etoposide-induced apoptosis of murine calvaria derived osteoblasts (data not shown). Further, E₂ acting via the ERα transactivated Elk-1 and that Elk-1 was required for the stimulation of SRE SEAP activity by the hormone (FIG. 5). Moreover, overexpression of a dn Elk-1 or a MAPK-transactivation inactive Elk-1 mutant, but not the wild type control, abrogated the anti-apoptotic effect of E₂, consistent with the effect of actinomycin D indicating that transcription is required for anti-apoptosis (FIG. 1C). The protective effect of E₂ on apoptosis was also abrogated by dn mutants of C/EBPβ or CREB. Lastly, the anti-apoptotic effect of E₂ was abolished by dn constructs for JNK1 or AP-1, but not a constitutive active JNK1, consistent with the finding that E₂ downregulates AP-1-SEAP activity. A dn fos did not interfere with the effect of E₂, indicating that only the c-jun component of AP-1 is required. The dn Elk-1, C/EBPb, CREB, JNK1 and AP-1 also abrogated the protective effect of E₂ or DHT in AR-transfected HeLa cells, as well as the protective effect of DHT in either AR- or ER-transfected cells (FIG. 6). The effects of dn Elk-1, C/EBPb, CREB, JNK1 and AP-1 were confirmed using the osteocytic MLO-Y4 cells which express endogenously ERα and β, but not AR (data not shown).

In full agreement with the portion of the data dealing with Elk-1, an ERK-mediated stimulation of SRE activity by E₂ has been demonstrated in other cell types (Duan et al, 2001; De Jager et al, 2001; Song et al, 2002), establishing that some “non-genotropic” actions lead to changes in gene transcription.

Kinases modulate cell survival not only through changes in gene transcription, but also through the modification of the functional activity of proteins, independent of any transcriptional changes (Bonni et al, 1999; Scheid and Duronio, 1998). Furthermore, there is evidence that either ERKs or PI3K induce the phosphorylation, and thereby inactivation, of the pro-apoptotic protein Bad (Yang et al, 1995; Scheid et al, 1999; Peruzzi et al, 1999; Lizcano et al, 2000). It was investigated whether the anti-apoptotic effect of estrogens, or 4-estren-3α,17β-diol, require convergence of these two signaling cascades on Bad. It was found that phosphorylation and inactivation of Bad was indispensable for the anti-apoptotic actions of E₂ or 4-estren-3α,17β-diol (FIG. 1D), as evidenced by the failure of HeLa cells expressing a dn Bad mutant that cannot be phosphorylated, to respond to either one of these two ligands. Furthermore, wortmannin, a PI3K inhibitor, but not SB203580, a p38 inhibitor, also attenuated the anti-apoptotic effect of E₂, as well as the effect of 4-estren-3α,17β-diol (FIG. 1D). In studies not shown here, it was also determined that PD98059, an inhibitor of ERKs, or wortmannin abolished E₂-induced phosphorylation of Bad (Kousteni et al, 2002). A diagrammatic representation of the kinase initiated signaling pathways and their downstream effectors required for the anti-apoptotic effects of sex steroids is depicted in the model of FIG. 1E.

Next, it was examined whether non-genotropic signals affect bone cells other than osteoblasts and whether their effects on bone cell survival might be sex non-specific. To do this, the effects of E₂, DHT, 4-estren-3α,17β-diol and the pyrazole were compared on osteoblast, as well as osteoclast survival, in murine bone cells from females and males. It was found that E₂, DHT or 4-estren-3α,17β-diol, but not the pyrazole, attenuated etoposide-induced apoptosis in primary cultures of osteoblasts in a dose dependent manner. Strikingly, however, by comparing here cell preparations from females or males, no difference was found in the potency of E₂, DHT or 4-estren-3α,17β-diol in cells from one sex versus the other (FIG. 7A). In sharp contrast to their effects on osteoblasts, E₂, DHT or 4-estren-3α,17β-diol, but not the pyrazole, stimulated osteoclast apoptosis (independent of whether stromal/osteoblastic support cells were present or absent from the cultures) in a dose dependent manner by as much as 3-fold. Once again, there was no difference in the potency of E₂, DHT or 4-estren-3α,17β-diol in cells from females and males (FIG. 7B). The effects of E₂ or DHT on the lifespan of osteoblasts or osteoclasts was blocked by either the ER antagonist ICI 182,780 or the AR antagonist flutamide; the same phenomenon was demonstrated in HeLa cells transfected with either ERα or the AR (not shown). Pre-treatment of osteoclasts for 1 hour prior to exposing them to E₂ or DHT with either U012345 or PP1, the specific inhibitors of ERK and Src phosphorylation, respectively, abrogated the pro-apoptotic effects of the sex steroids and 4-estren-3α,17β-diol (FIG. 8). This finding strongly suggests that as in the case of the anti-apoptotic effects of the sex steroids and 4-estren-3α,17β-diol on osteoblasts, their pro-apoptotic effects on osteoclasts require activation of the Src/ERK signaling pathway.

Because of the in vitro evidence of anti-apoptotic effects of estrogens and androgens on murine osteoblasts and pro-apoptotic effects on osteoclasts from females and males and an interchangeable ligand/receptor interaction mediating ERK activation, the sex specificity of the skeletal effects of E₂ and DHT was compared in sex steroid deficient mice. For these studies, mature Swiss Webster mice (8 month old), n=8-10 per group, were sham-operated or gonadectomized (GNDX). The GNDX animals were then left untreated or were treated with slow release pellets containing E₂ or DHT, at doses corresponding to physiologic replacement, as determined by the minimal dose needed to restore uterine or seminal vesicle weight in gonadectomized females and males. Four and/or six weeks later, osteoblast apoptosis in histologic sections of the vertebrae, bone mineral density (BMD), osteoblastogenesis and osteoclastogenesis in ex vivo bone marrow cultures, serum osteocalcin, and wet uterine or seminal vesicle weight were determined. Ovariectomy (OVX) or orchidectomy (ORX) increased the prevalence of osteoblast apoptosis (FIGS. 7C & 7D) and caused loss of BMD (FIGS. 7E & 7F). Likewise, gonadectomy upregulated osteoblastogenesis and osteoclastogenesis (FIG. 9). All these changes were effectively prevented by either E₂ or DHT replacement, irrespective of the sex of the mouse. In contrast to the equivalence of the skeletal actions of E₂ and DHT, E₂ administration to ORX males failed to restore seminal vesicle weight (FIG. 7F); however, DHT administration to OVX females did restore wet uterine weight (FIG. 7E), probably because of the 300-fold higher dose of DHT as compared to E₂. A uterotrophic effect of high DHT doses has been demonstrated previously in the rat (Tobias et al, 1994).

Having established in vitro that 4-estren-3α,17β-diol selectively activates non-genotropic function(s) of the ER or the AR, without affecting classical transcription, the effects of this compound in vivo were compared to those of E₂ or DHT on bone and on reproductive tissues from OVX or ORX Swiss Webster mice. Specifically, 4-estren-3α,17β-diol at a dose of 7.6 mg per 60 day slow release pellet, was compared to a replacement dose of either E₂ or DHT defined above. The ˜300-fold higher dose of 4-estren-3α,17β-diol, as compared to E₂, in these experiments was decided based on its lower binding affinity for the ER (FIG. 10). The 4-estren-3α,17β-diol had no effect on body weight. Strikingly, 4-estren-3α,17β-diol was as effective, if not superior to estradiol on global and spinal BMD in females (see statistical anlysis in the additional details of experimental procedures). Even more remarkably, OVX mice receiving 4-estren-3α,17β-diol consistently exhibited greater BMD change in the hindlimb, not only compared to the OVX mice receiving E₂ replacement but also compared to the estrogen replete sham controls, indicating an anabolic effect, i.e. addition of new bone, at this site of predominantly cortical bone (FIGS. 11A and B). The 4-estren-3α,17β-diol also appeared at least as effective if not superior to DHT replacement in ORX mice, as BMD values in the spine of ORX+4-estren-3α,17β-diol group, but not the ORX+DHT, were significantly higher than the untreated ORX group (FIG. 11C). In line with the contention that 4-estren-3α,17β-diol was at least as effective as estradiol in the spine, bone compression strength in the vertebrae of the 4-estren-3α,17β-diol-treated female mice was greater than in mice receiving E₂. In the male mice, however, 4-estren-3α,17β-diol and DHT were equally effective (FIG. 12A). Importantly, in contrast to a pharmacologic dose of E₂ (100× the replacement dose for mice), which caused the expected undesirable effect of closing the bone marrow cavity, 4-estren-3α,17β-diol had no adverse effects on the marrow cavity (FIG. 12B). Consistent with its in vitro properties, 4-estren-3α,17β-diol prevented the increased prevalence of osteoblast and osteocyte apoptosis in the lumbar vertebrae of gonadectomized females or males (FIG. 12C).

To obtain clues for possible cellular mechanisms accounting for the distinct profile of the skeletal effects of the 4-estren-3α,17β-diol versus E₂, histomorphometric analysis of the distal femoral metaphysis and lumbar vertebrae (L1-L4) was performed. Histomorphometric variability was very high in the limited amount of cancellous tissue available in the femora after ovariectomy, making it impractical for the purpose of this analysis (not shown). By contrast, sample variability was limited when the 4 lumbar vertebrae which collectively contain eight times as much cancellous tissue as the distal femoral metaphysis were used. Compared to OVX mice treated with E₂, mice receiving 4-estren-3α,17β-diol had significantly greater cortical and trabecular width; 27.8% and 33.9%, respectively. (FIGS. 13A & B). Most strikingly, the number of osteoblasts on the trabeculae of the 4-estren-3α,17β-diol-treated mice was greater (319%) than that in the E₂-treated group (FIG. 13C); and consistent with this, the unmineralized matrix produced by osteoblasts (osteoid perimeter) was also increased by 270% (FIG. 13D). The rate of bone formation (FIG. 13E) and osteoclast number (FIG. 13F) was suppressed by either E₂ or the 4-estren-3α,17β-diol as compared to the OVX group. These findings confirm the adequacy of the E₂ replacement dose in suppressing the ovariectomy-induced increase in bone turnover and support the BMD findings. Lastly, in line with the BMD data and the higher osteoblast number in the 4-estren-3α,17β-diol treated mice, serum osteocalcin—a biochemical index of osteoblast number—was significantly higher in two separate experiments not only compared to OVX+E₂ but also to the estrogen replete sham controls (FIG. 13G).

Most remarkably, considering its effects on bone, unlike E₂ or DHT, 4-estren-3α,17β-diol had no effect on the uterine or the seminal vesicle weight of the gonadectomized mice (FIGS. 14A & B). The lack of an effect of 4-estren-3α,17β-diol on reproductive tissues was confirmed by histologic analysis of the uterus (FIG. 14C). Lastly, unlike E₂ or the pyrazole, the 4-estren-3α,17β-diol did not stimulate MCF-7 cell proliferation (FIG. 14D). None of the three ligands affected the proliferation of the ER negative MDA-MB-2321 cells (data not shown).

A system for the rapid screening of compounds for ANGELS activity is illustrated in FIG. 15. In FIG. 15A are shown the results of a competitive radiometric binding assay through which the affinity of ten compounds that are related to 4-estrene-3α,17β-diol for both estrogen receptor alpha (ERα) and estrogen receptor beta (ERβ) is determined (Carlson, et al., 1997). The affinities are reported as Relative Binding Affinity (RBA) values, which is essentially a percent scale relative to the affinity of the binding standard estradiol (RBA for estradiol is 100). Referring to the compound numbers in FIG. 15A, it can be seen that only compounds 1, 5, 9, and 10 have substantial affinity for either ERα or ERβ, that is, RBA values that are greater than 0.1 for either receptor; the other six compound have RBA values less than 0.1.

These same ten compounds are screened for their genotropic activity (FIG. 15B) in a reporter gene assay (Kousteni et al. 2001) at a single concentration of 10⁻⁸ M. (FIG. 15B, upper panel). From this screen, it is evident that five compounds (1, 5, 6, 9, and 10) show substantial genotropic activity, that is, they activate the reporter gene to levels above that for vehicle control (Veh) and 4-estrene-3α,17β-diol (4-ED), and in some cases (1, 9, and 10) to levels approaching that of estradiol (E₂). Therefore, these compounds are not ANGELS compounds, because they show substantial genotropic activity. The five compounds (2, 3, 4, 7, 8) that showed minimal to no activity in the reporter gene assay were further examined for their antiapoptotic activity (FIG. 15B, lower panel) in a dose response assay. All five of these were found to have high potency in reversing etoposide-induced apoptosis (Kousteni et al. 2001), and are considered to be ANGELS.

Additional Details of Experimental Procedures

Plasmids: SRE- and AP-1-SEAP were purchased from Clontech Laboratories (Palo Alto, Calif.). ElkC and ElkC383/389 and dn Elk-1 were obtained from S. Safe, Texas A & M University (Duan et al, 2001). GAL4-luc was obtained from M. Karin, University of California, San Diego (Tian and Karin, 1999). Construction of the human ERα ligand binding domain (E), E-Mem and E-Nuc mutants and the cDNAs for wt Src and SrcK295M (Src K⁻), wild type (wt) or She mutants and dn MEK were previously described (Kousteni et al, 2001). JNK1 and dn JNK1 were obtained from R. J. Davis, University of Massachusetts (Whitmarsh et al, 1995). A BAD mutant in which serines 112, 136, and 155 were mutated to alanine (AAA) was provided by X-M Zhou (Apoptosis Technology, Inc. Cambridge, Mass.) (Zhou et al, 2000). Dn CREB and dn C/EBPβ were provided by C. Vinson (National Cancer Institute, National hIstitutes of Health, Bethesda, Md.) (Ahn et al, 1998). Dn AP-1 (TAM67) was provided by T. Chambers (University of Arkansas for Medical Sciences, Little Rock, Ak.) (Brown et al, 1994).

Transient transfections and reporter assays: HeLa cells were transfected using Lipofectamine Plus (Life Technologies Inc.). For reporter assays (luciferase or secreated alkaline phosphatase SEAP), serum-starved cells were treated with the indicated steroids for 15 min after which the steroid-containing media were removed, cells washed twice with 1% BSA in PBS, and fresh media without steroid were added. SEAP or luciferase assays were performed 6 h later using the Great EscAPe SEAP Chemiluminescence Kit (Clontech, Palo Alto, Calif.) or the dual luciferase Kit (Promega, Madison, Wis.), respectively, according to the manufacturer's instructions. Both reporter activities were normalized to renilla luciferase activity.

MCF-7 cell proliferation assay: MCF-7 cells were serum-starved in the presence of 10⁻⁸ M ICI 182,780 for 96 h, after which the ICI-containing medium was replaced with medium-containing vehicle or 10⁻¹²-10⁻⁷ M of the indicated steroids for an additional 48 h. At that time, proliferation was assayed by measuring ³H-Thymidine uptake as previously described (Bellido et al, 1997).

Quantification of apoptotic cells in vitro: Apoptosis of HeLa cells or calvaria-derived osteoblastic cells was quantified by direct visualization of changes in nuclear morphology or by trypan blue staining, respectively, as previously described (Kousteni et al, 2001). Apoptosis of osteoclasts, derived from bone marrow cells cultured with 30 ng/ml M-CSF and 30 ng/ml soluble RANK ligand, was quantified by measuring caspase 3 activity as previously described (Weinstein et al, 2002).

Bone densitometry, histomorphometry, osteoblast apoptosis in bone sections, vertebral compression testing, and osteocalcin measurements: Bone mineral density (BMD) of live mice by DEXA, static and dynamic histomorphometric analysis, and osteoblast apoptosis by in situ nick-end labeling (ISEL) of undecalcified bone sections, were performed as previously described (Weinstein et al, 2002). Bone compression strength was measured using a single column material testing machine, a calibrated tension/compression load cell and Merlin IX analysis software (Model 5542, Instron Corp., Canton, Mass.). The fifth lumbar vertebrae were cleaned of surrounding soft tissue, wrapped in gauze soaked in 37°±0.5 C normal saline and tested on the day of sacrifice as described (Weinstein, 2000). Length, width and depth of the bones were recorded with a digital caliper at a resolution of 0.01 mm (Mitutoyo #500-196, Ace Tools, Ft. Smith, Ak.). The cross-sectional area was assumed to be an ellipse and calculated as A=0.25π (width)(depth). Articular and spinous processes that would interfere with compression were excised using an iris scissors. After pre-seating with less than 0.5 Newtons of applied load, vertebrae were compressed between screw-driven loading platens using a lower-platen, miniature spherical seat that minimized shear by adjusting to irregularities in the end plates of the specimens. Best seating was obtained when the load was applied along the caudocephald axis at a speed of 0.5 mm/min until failure. The maximum load and displacement were recorded and ultimate strength or stress was calculated from the compression measurements and vertebral dimensions. Serum osteocalcin concentration was determined by radioimmunoassay (Biomedical Technologies Inc., Stoughton, Mass.), as previously described (Jilka et al, 1998).

Quantification of osteoblast and osteoclast precursors: The number of osteoblast and osteoclast precursors obtained from murine femoral marrow cells was determined as previously described (Jilka et al, 1998).

Statistical analysis: ANOVA was used to detect treatment effects. Specifically, in FIGS. 1-6 & 8, Dunnett's test (Kuehl et al, 2000) was used to detect differences between various treatments as compared to the vehicle control group. To detect differences in the efficacy of the various compounds shown in FIGS. 7A & B, the dose response curves were compared using tests for linear trend (Kuehl, 2002). Bonferroni's method was used to perform all pairwise comparisons of treatment groups in FIGS. 7E & F, FIGS. 11A, B, C, FIG. 12A, FIG. 13, FIGS. 14A and B and FIG. 9. Because the normality assumption was not satisfied for the data in FIGS. 3C & D and FIG. 12C, Wilcoxon's rank sum test (Steel et al, 1997) was used to perform all pairwise comparisons of treatment groups using a Bonferroni correction. In FIG. 14D, a two-way ANOVA was used to detect treatment and dose effects and tests for linear trend were subsequently employed to determine significant effects of each compound. In FIG. 10, logistic regression was used to estimate EC50, which was then used to determine the relative binding affinity. There were no significant differences in BMD, serum osteocalcin, and uterine weight measurements between the two experiments involving the 6 and 8 month old female mice, by 2-way ANOVA. Furthermore, there was no significant difference among treatment effects across the 2 experiments. Therefore, the data corresponding to these measurements in the two experiments were pooled and are shown as such in FIGS. 11B, 13G, and 14A.

All literature references and patents mentioned herein are hereby incorporated by reference in their entireties. Although the foregoing invention has been described in terms of certain preferred embodiments, other embodiments will become apparent to those of ordinary skill in the art in view of the disclosure herein. Accordingly, the present invention is not intended to be limited by the recitation of preferred embodiments.

### REFERENCES

The following references are hereby incorporated by reference in their entireties:

WO 00/19823

WO 00/20007

WO 00/20625

WO 00/28982

WO 01/96605

Ahn S. et al., Mol. Cell Biol. 18, 967-977 (1998).

Anstead, G. M., Carlson, K. E. and Katzenellenbogen, J. A. (1997) The estradiol pharmacophore: ligand structure-estrogen receptor binding affinity relationships and a model for the receptor binding site. Steroids, 62, 268-303.

Bellido, T., Borba, V. Z., Roberson, P., Manolagas, S. C. (1997) Endocrinology 138, 3666-3676.

Bilezikian, J. P., A. Morishima, J. Bell, and M. M. Grumbach. (1998) Increased bone mass as a result of estrogen therapy in a man with aromatase deficiency. N. Engl. J Med. 339:599-603.

Bindal, R. D., Carlson, K. E., Reiner, G. C. and Katzenellenbogen, J. A. (1987) 11β-Chloromethyl-[³H]estradiol-17β-estradiol: A very high affinity, reversible ligand for the estrogen receptor. J. Steroid Biochem., 28, 361-370.

Bonni, A., A. Brunet, A. E. West, S. R. Datta, M. A. Takasu, and M. E. Greenberg. (1999) Cell survival promoted by the Ras-MAPK signaling pathway by transcription-dependent and -independent mechanisms [see comments]. Science 286:1358-1362.

Boyden, L. M., J. Mao, J. Belsky, L. Mitzner, A. Farhi, M. A. Mitnick, D. Wu, K. Insogna, and R. P. Lifton. (2002) High bone density due to a mutation in LDL-receptor-related protein 5. The New England Journal of Medicine 346:1513-1521.

Brown, P. H., Chen, T. K., Birrer, M. J. (1994) Oncogene 9, 791-799.

Buck, M., V.Poli, G. P. van der, M. Chojkier, and T. Hunter. (1999) Phosphorylation of rat serine 105 or mouse threonine 217 in C/EBP beta is required for hepatocyte proliferation induced by TGF alpha. Mol. Cell 4:1087-1092.

Carlson, K. E., Choi, I., Gee, A., Katzenellenbogen, B. S., and J. A. Katzenellenbogen, Altered Ligand Binding Properties and Enhanced Stability of a Constitutively Active Estrogen Receptor: Evidence That an Open Pocket Conformation Is Required for Ligand Interaction. Biochemistry, 1997, 36, 14897-14905.

Chen, J. -R., S. Kousteni, L. Han, A. M. Vertino, L. K. McCauley, A. A. Ali, T. Bellido, R. S. Weinstein, C. A. O'Brien, R. L. Jilka, and S. C. Manolagas. (2002) Interchangeable ligand/receptor interactions mediate ERK activation and the pro- and anti-apoptotic effects of estrogens and androgens on murine osteoclasts and osteoblasts from females and males: a molecular explanation of the in vivo equivalence of their skeletal actions in either sex. J Bone Miner. Res., abstract in press.

Chen, Z., I. S. Yuhanna, Z. Galcheva-Gargova, R. H. Karas, M. E. Mendelsohn, and P. W. Shaul. (1999) Estrogen receptor alpha mediates the nongenomic activation of endothelial nitric oxide synthase by estrogen. J. Clin. Invest. 103:401-406.

Chi, D. Y., Wilson, S. R. and J. A. Katzenellenbogen, Crystal Structure of Doisynolic Acid and the Structure of Other Products Formed During its Synthesis. Steroids 1995, 60, 261-264.

Croniger, C. M., C. Millward, J. Yang, Y. Kawai, I. J. Arinze, S. Liu, M. Harada-Shiba, K. Chakravarty, J. E. Friedman, V. Poli, and R. W. Hanson. (2001) Mice with a Deletion in the Gene for CCAAT/Enhancer-binding Protein beta Have an Attenuated Response to cAMP and Impaired Carbohydrate Metabolism. J. Biol. Chem. 276:629-638.

Cruzalegui, F. H., E. Cano, and R. Treisman. (1999) ERK activation induces phosphorylation of Elk-1 at multiple S/T-P motifs to high stoichiometry. Oncogene 18:7948-7957.

De Jager, T., T. Pelzer, S. Mueller-Botz, A. Imam, J. Muck, and L. Neyses. (2001) Mechanisms of estrogen receptor action in the myocardium: rapid gene activation via the ERK1/2 pathway and serum response elements. J. Biol. Chem.

Djerassi, C. (1963) Steroid Reactions: An outline for organic chemists. Holden-Day, San Francisco, Calif.

Doran, P. M., B. L. Riggs, E. J. Atkinson, and S. Khosla. (2001) Effects of raloxifene, a selective estrogen receptor modulator, on bone turnover markers and serum sex steroid and lipid levels in elderly men. J Bone Miner. Res. 16:2118-2125.

Duan, R., W. Xie, R. C. Burghardt, and S. Safe. (2001) Estrogen receptor-mediated activation of the serum response element in MCF-7 cells through MAPK-dependent phosphorylation of Elk-1. J. Biol. Chem. 276:11590-11598.

Dupont, S., A. Krust, A. Gansmuller, A. Dierich, P. Chambon, and M. Mark. (2000) Effect of single and compound knockouts of estrogen receptors alpha (ERalpha) and beta (ERbeta) on mouse reproductive phenotypes. Development 127:4277-4291.

Eicher, T. and Hauptmann, S. (1995) The Chemistry of Heterocycles. Thieme, New York, N.Y.

Falkenstein, E., H. C. Tillmann, M. Christ, M. Feuring, and M. Wehling. (2000) Multiple actions of steroid hormones—a focus on rapid, nongenomic effects. Pharmacol. Rev. 52:513-556.

Fieser, L. F. and Fieser, M. (1967) Steroids. Reinhold, New York, N.Y.

Fink, B. E., Mortensen, D. S., Stauffer, S. R., Aron, Z. D. and Katzenellenbogen, J. A. (1999) Novel Structural Templates for Estrogen-Receptor Ligands and Prospects for Combinatorial Synthesis of Estrogens. Chem. Biol., 6, 205-219.

French, A. N., Napolitano, E., VanBrocklin, H. F., Hanson, R. N., Welch, M. J. and Katzenellenbogen, J. A. (1993a) Synthesis, radiolabeling and tissue distribution of 11β-fluoroalkyl- and 11β-fluoroalkoxy-substituted estrogens: Target tissue uptake selectivity and defluorination of a homologous series of fluorine-18-labeled estrogens. Nucl. Med. Biol., 20, 31-47.

French, A. N., Wilson, S. R., Welch, M. J. and Katzenellenbogen, J. A. (1993b) A synthesis of 7α-substituted estradiols: Synthesis and biological evaluation of a 7α-pentyl-substituted BODIPY fluorescent conjugate and a fluorine-18-labeled 7α-pentylestradiol analog. Steroids, 58, 157-169.

Fried, J. and Edwards, J. A. (1972) Organic Reactions in Steroid Chemistry. 1-2. van Nostrand-Reinhold, New York, N.Y.

Gao, H., Katzenellenbogen, J. A., Garg, R. and Hansch, C. (1999) Comparative QSAR Analysis of Estrogen Receptor Ligands. Chem. Rev., 99, 723-744.

Gentile, M. A., H. Zhang, S. Harada, G. A. Rodan, and D. B. Kimmel. (2001) Bone response to estrogen replacement in ovx double estrogen receptor-(α and β) knockout mice. J Bone Miner. Res 16:S146.

Gilchrist, T. L. (1992) Heterocyclic Chemistry, Third Edition. Addison-Wesley Longman, Edinburgh, UK.

Grotewold, L. and U. Rüther. (2002) The Wnt antagonist Dickkopf-1 is regulated by Bmp signaling and c-Jun and modulates programmed cell death. The EMBO Journal 21:966-975.

Grundy, J. (1956) Artificial Estrogens. Chem. Res., 19, 281-416.

Gupta, R. R., Kumar, M. and Gupta, V. (1999) Heterocyclic Chemistry: Five-Membered Heterocycles. 2. Springer Verlag, Berlin, Germany.

Hall, J. M., J. F.Couse, and K. S. Korach. (2001) The Multifaceted Mechanisms of Estradiol and Estrogen Receptor Signaling. J. Biol. Chem. 276:36869-36872.

Herrington, D. M., D. M. Reboussin, K. B. Brosnihan, P. C. Sharp, S. A. Shumaker, T. E. Snyder, C. D. Furberg, G. J. Kowalchuk, T. D. Stuckey, W. J. Rogers, D. H. Givens, and D. Waters. (2000) Effects of estrogen replacement on the progression of coronary-artery atherosclerosis. N. Engl. J. Med. 343:522-529.

Hill, C. S. and R. Treisman. (1995) Transcriptional regulation by extracellular signals: Mechanisms and specificity. Cell 80:199-211.

Hodgin, J. B., J. W. Knowles, H. S. Kim, O. Smithies, and N. Maeda. (2002) Interactions between endothelial nitric oxide synthase and sex hormones in vascular protection in mice. J. Clin. Invest. 109:541-548.

Hodgin, J. B., J. H. Krege, R. L. Reddick, K. S. Korach, O. Smithies, and N. Maeda. (2001) Estrogen receptor a is a maior mediator of 17b-estradiol's atheroprotective effects on lesion size in Apoe−/− mice. J. Clin. Invest. 107:333-340.

Jilka R. L. et al. (1998) J. Clin.Invest. 101, 1942-1950.

Joule, J. A., Mills, K. and Smith, G. F. (1995) Heterocyclic Chemistry, Third Edition. Chapman & Hall, London, UK.

Katzenellenbogen, J. A., B. W. O'Malley, and B. S. Katzenellenbogen. (1996) Tripartite steroid hormone receptor pharmacology: interaction with multiple effector sites as a basis for the cell- and promoter-specific action of these hormones. Mol. Endocrinol. 10:119-131.

Khosla, S., L. J. Melton, III, E. J. Atkinson, W. M. O'Fallon, G. G. Klee, and B. L. Riggs. (1998) Relationship of serum sex steroid levels and bone turnover markers with bone mineral density in men and women: A key role for bioavailable estrogen. J Clin Endocrinol & Metab 83:2266-2274.

King, W. J. and G. L. Greene. (1984) Monoclonal antibodies localize oestrogen receptor in the nuclei of target cells. Nature 307:745-747.

Kirk, D. N. and Hartshorn, M. P. (1968) Reaction Mechanisms in Organic Chemistry: Steroid Reaction Mechanisms (Monograph 7). Elsevier, Amsterdam, NL.

Komesaroff, P. A., M. Fullerton, M. D. Esler, A. Dart, G. Jennings, and K. Sudhir. (2001) Low-dose estrogen supplementation improves vascular function in hypogonadal men. Hypertension 38:1011-1016.

Kousteni, S., T. Bellido, L. I. Plotkin, C. A. O'Brien, D. L. Bodenner, K. Han, G. DiGregorio, J. A. Katzenellenbogen, B. S. Katzenellenbogen, P. K. Roberson, R. S. Weinstein, R. L. Jilka, and S. C. Manolagas. (2001). Nongenotropic, sex-nonspecific signaling through the estrogen or androgen receptors: dissociation from transcriptional activity. Cell 104:719-730.

Kousteni, S., L. Han, T. Bellido, C. A. O'Brien, R. L. Jilka, and S. C. Manolagas. (2002) Estrogens induce Bad phosphorylation via both ERK and PI3K activation: a two pronged signal requirement for their anti-apoptotic effects. J Bone Miner. Res., in press

Kuehl, R. O. (2000) Statistical Principles of Research Design and Analysis (Duxbury Press, New York, ed. 2nd), pp. 104-107.

Kuiper, G. G., E. Enmark, M. Pelto-Huikko, S. Nilsson, and J. A. Gustafsson. (1996) Cloning of a novel receptor expressed in rat prostate and ovary. Proc. Natl. Acad. Sci. USA 93:5925-5930.

Kushner, P. J., D. A. Agard, G. L. Greene, T. S. Scanlan, A. K. Shiau, R. M. Uht, and P. Webb. (2000) Estrogen receptor pathways to AP-1. J. Steroid Biochem. Mol. Biol. 74:311-317.

Larock, R. C. (1999) Comprehensive Organic Transformations: A guide to functional group preparations, Second Edition. VCH, New York, N.Y.

Levin, E. R. (2001) Cell localization, physiology, and nongenomic actions of estrogen receptors. J. Appl. Physiol. 91:1860-1867.

Lewis, D. A., M. P. Bracamonte, K. S. Rud, and V. M. Miller. (2001) Selected contribution: Effects of sex and ovariectomy on responses to platelets in porcine femoral veins. J. Appl. Physiol. 91:2823-2830.

Lin, X. and Huebner, V. (2000) Non-steroidal ligands for steroid hormone receptors. Curr. Opin. Drug Disc., 3, 383-398.

Lizcano, J. M., N. Morrice, and P. Cohen. (2000) Regulation of BAD by cAMP-dependent protein kinase is mediated via phosphorylation of a novel site, Ser155. Biochem. J. 349:547-557.

Magarian, R. A., Overacre, L. B., Singh, S. and Meyer, K. L. (1994) The medicinal chemistry of nonsteroidal antiestrogens: A review. Curr. Med. Chem., 1, 61-104.

Mangelsdorf, D. J., C. Thummel, M. Beato, P. Herrlich, G. Schüitz, K. Umesono, B. Blumberg, P. Kastner, M. Mark, P. Chambon, and R. M. Evans. (1995) The nuclear receptor superfamily: The second decade. Cell 83:835-839.

Manolagas, S. C. (2000) Birth and death of bone cells: basic regulatory mechanisms and implications for the pathogenesis and treatment of osteoporosis. Endocr. Rev. 21:115-137.

Manolagas, S. C. and S. Kousteni. (2001) Non-reproductive sites of action of reproductive hormones. Endocrinology 2200-2204.

Manolagas, S. C., S. Kousteni, and R. L. Jilka. (2002) Sex steroids and bone. Recent Prog. Horm. Res. 57:385-409.

Manson, J. E. and K. A.Martin. (2001) Clinical practice. Postmenopausal hormone-replacement therapy. [Review] [34 refs]. N. Engl. J. Med. 345:34-40.

Marais, R., Wynne, J., Treisman, R., (1993) Cell 73, 381-393.

McElvany, K. D., Carlson, K. E., Welch, M. J., Senderoff, S. G. and Katzenellenbogen, J. A. (1982) In vivo comparison of 16α[⁷⁷Br]bromoestradiol-17β and 16α-[¹²⁵I]iodoestradiol-17β. J Nuc. Med., 23, 420-424.

McEwen, B. S. and S. E. Alves. (1999) Estrogen actions in the central nervous system. Endocr. Rev. 20:279-307.

McKenna, N. J. and B. W. O'Malley. (2002) Combinatorial control of gene expression by nuclear receptors and coregulators. Cell 108:465-474.

Mortensen, D. S., A. L. Rodriguez, K. E. Carlson, J. Sun, B. S. Katzenellenbogen, J. A. Katzenellenbogen. Synthesis and Biological Evaluation of a Novel Series of Furans: Ligands Selective for Estrogen Receptor Alpha. J. Med Chem., 2001, 8, 3838-3848.

Moggs, J. G. and G. Orphamides. (2001) Estrogen receptors: orchestrators of pleiotropic cellular responses. [Review] [53 refs]. EMBO Reports 2:775-781.

Mosca, L., P. Collins, D. M. Herrington, M. E. Mendelsohn, R. C. Pastemak, R. M. Robertson, K. Schenck-Gustafsson, S. C. Smith, Jr., K. A. Taubert, N. K. Wenger, and American Heart Association. (2001) Hormone replacement therapy and cardiovascular disease: a statement for healthcare professionals from the American Heart Association. Circulation 104:499-503.

Ojasoo, T. and Raynaud, J. P. (1978) Unique steroid congeners and receptors studies. Cancer Res., 38, 4186-4198.

Ott, S. M., A. Oleksik, Y. Lu, K. Harper, and P. Lips. (2002) Bone histomorphometric and biochemical marker results of a 2-year placebo-controlled trial of raloxifene in postmenopausal women. J Bone Miner. Res 17:341-348.

Paquette, L. A., Editor-in-Chief (1995) Encyclopedia of Reagents for Organic Synthesis. 1-8. John Wiley, New York, N.Y.

Peruzzi, F., M. Prisco, M. Dews, P. Salomoni, E. Grassilli, G. Romano, B. Calabretta, and R. Baserga. (1999) Multiple Signaling Pathways of the Insulin-Like Growth Factor 1 Receptor in Protection from Apoptosis. Mol. Cell. Biol. 19:7203-7215.

Pietras, R. J. and C. M.Szego. (1977) Specific binding sites for oestrogen at the outer surfaces of isolated endometrial cells. Nature 265:69-72.

Pomper, M. G., VanBrocklin, H., Thieme, A. M., Thomas, R. D., Kiesewetter, D. O., Carlson, K. E., Mathias, C. J., Welch, M. J. and Katzenellenbogen, J. A. (1990) 11β-Methoxy-, 11β-ethyl- and 17α-ethynyl-substituted 16α-fluoroestradiols: Receptor-based imaging agents with enhanced uptake efficiency and selectivity. J. Med. Chem., 33, 3143-3155.

Quigley, C. A., A. De Bellis, K. B. Marschke, M. K. el Awady, E. M. Wilson, and F. S. French. (1995) Androgen receptor defects: historical, clinical, and molecular perspectives. Endocr. Rev. 16:271-321.

Raynaud, J. P., Martin, P. M., Bouton, M. M. and Ojasoo, T. (1978) 11beta-Methoxy-17-ethynyl-1,3,5(10)-estratriene-3,17beta-diol (moxestrol), a tag for estrogen receptor binding sites in human tissues. Cancer Res, 38, 3044-50.

Reichardt, H. M., K. H.Kaestner, J. Tuckermann, O. Kretz, O. Wessely, R. Bock, P. Gass, W. Schmid, P. Herrlich, P. Angel, and G. Schütz. (1998) DNA binding of the glucocorticoid receptor is not essential for survival. Cell 93:531-541.

Santoro, N. F., N. F. Col, M. H. Eckman, J. B. Wong, S. G. Pauker, J. A. Cauley, J. Zmuda, S. Crawford, C. B. Johannes, J. E. Rossouw, and C. N. Merz. (1999) Therapeutic controversy: Hormone replacement therapy—where are we going?. J Clin Endocrinol & Metabol 84:1798-1812.

Sasson, S. and Katzenellenbogen, J. A. (1989) Reversible, positive cooperative interaction of 11β-chloromethyl-[³H]estradiol-11β with the calf uterine estrogen receptor. J. Steroid Biochem., 33, 859-865.

Scheid, M. P. and V. Duronio. (1998) Dissociation of cytokine-induced phosphorylation of Bad and activation of PKB/akt: involvement of MEK upstream of Bad phosphorylation. Proc. Natl. Acad. Sci. U.S. A 95:7439-7444.

Scheid, M. P., K. M. Schubert, and V. Duronio. (1999) Regulation of bad phosphorylation and association with Bcl-x(L) by the MAPK/Erk kinase. J. Biol. Chem. 274:31108-31113.

Schmidt, W. N. and Katzenellenbogen, B. S. (1979) Androgen-uterine interactions: An assessment of androgen interaction with the testosterone and estrogen receptor systems and stimulation of uterine growth and progesterone receptor synthesis. Mol. Cell. Endocrinol., 15, 91-108.

Schmidt, W. N., Sadler, M. A. and Katzenellenbogen, B. S. (1976) Androgen-uterine interaction: Nuclear translocation of the estrogen receptor and induction of the synthesis of the uterine induced protein (IP) by high concentrations of androgens in vitro but not in vivo. Endocrinology, 98, 702-716.

Scribner, A. W., Jonson, S. D., Welch, M. J. and Katzenellenbogen, J. A. (1997) Synthesis, estrogen receptor binding, and tissue distribution of [¹⁸F]fluorodoisynolic acids. Nucl. Med. Biol., 24, 209-224.

Shoppee, C. W. (1964) Chemistry of the Steroids, Second Edition. Butterworths, London, UK.

Simoncini, T., A. Hafezi-Moghadam, D. P. Brazil, K. Ley, W. W. Chin, and J. K. Liao. (2000) Interaction of oestrogen receptor with the regulatory subunit of phosphatidylinositol-3-OH kinase Nature 407:538-541.

Smith, M. B. and March, J. (2001) March's Advanced Organic Chemistry: Reactions, mechanisms, and structure, 5th Edition. Wiley-Interscience, New York, N.Y.

Solmssen, U. V. (1945) Synthetic estrogens and the relation between their structure and their activity. Chem. Res., 37, 481-598.

Song, R. X., R. A. McPherson, L. Adam, Y. Bao, M. Shupnik, R. Kumar, and R. J. Santen. (2002) Linkage of rapid estrogen action to MAPK activation by ERalpha-Shc association and Shc pathway activation. Mol. Endocrinol. 16:116-127.

Stauffer, S. R., Coletta, C. J., Tedesco, R., Sun, J., Katzenellenbogen, B. S. and Katzenellenbogen, J. A. (2000) Pyrazole Ligands: Structure-Affinity/Activity Relationships Of Estrogen Receptor-a Selective Agonists. J. Med. Chem., 43, 4934-4947.

Stauffer, S. R., Huang, Y. R., Aron, Z. D., Coletta, C. J., Sun, J., Katzenellenbogen, B. S. and Katzenellenbogen, J. A. (2001a) Triarlpyrazoles with Basic Side Chains: Development of Pyrazole-Based Estrogen Receptor Antagonists. Bio. Med. Chem., 9, 151-161.

Stauffer, S. R., Huang, Y. R., Coletta, C. J., Tedesco, R. and Katzenellenbogen, J. A. (2001b) Estrogen Pyrazoles: Defining the Pyrazole Core Structure and the Orientation of Substituents in the Ligand Binding Pocket of the Estrogen Receptor. Bio. Med. Chem., 9, 141-150.

Stauffer, S. R. and Katzenellenbogen, J. A. (2000a) Acyclic Amides as Estrogen Receptor Ligands: Synthesis, Binding, Activity, and Receptor Interaction. Bio. Med. Chem., 8, 1293-1316.

Stauffer, S. R. and Katzenellenbogen, J. A. (2000b) Solid-Phase Synthesis of Tetrasubstituted Pyrazoles, Novel Ligands for the Estrogen Receptor. J. Combinat. Chem., 2, 318-329.

Steel, R. G. D., Torrie, J. H., Dickey, D. A. (1997) Principles and Procedures of Statistics: A Biometrical Approach (McGraw-Hill, New York, ed. 3rd,), pp. 569-570.

Sun, J., Meyers, M. J., Fink, B. E., Rajendran, R., Katzenellenbogen, J. A. and Katzenellenbogen, B. S. (1999) Novel Ligands that Function as Selective Estrogens or Antiestrogens for Estrogen Receptor-β or Estrogen Receptor-β. Endocrinology, 140, 800-804.

Tedesco, R., Katzenellenbogen, J. A. and Napolitano, E. (1997a) 7α,11β-Disubstituted estrogens: Probes for the shape of the ligand binding pocket in the estrogen receptor. Bioorg. Med. Chem. Lett., 7, 2919-2924.

Tedesco, R., Katzenellenbogen, J. A. and Napolitano, E. (1997b) An expeditious route to 7α-substituted estradiol derivatives. Tetrahedron Lett., 38, 7997-8000.

Tian J. and Karin M. (1999), J. Biol.Chem. 274, 15173-15180.

Trost, B. M., Editor-in-Chief and Fleming, I., Deputy Editor-in-Chief (1991) Comprehensive Organic Synthesis: Selectivity, strategy & efficiency in modem organic chemistry. 1-9.

Tobias, J. H., A. Gallagher, and T. J. Chambers. (1994) 5α-dihydrotestosterone partially restores cancellous bone volume in osteopenic ovariectomized rats. Am. J. Physiol. Endocrinol. Metab. 267:E853-E859.

Toran-Allerand, C. D., M. Singh, and G. J. Setalo. (1999) Novel mechanisms of estrogen action in the brain: new players in an old story. Front. Neuroendocrinol. 20:97-121.

Treisman, R. (1996) Regulation of transcription by MAP kinase cascades. [Review] [95 refs]. Curr. Opin. Cell Biol. 8:205-215.

Valverde, M. A., P. Rojas, J. Amigo, D. Cosmelli, P. Orio, M. I. Bahamonde, G. E. Mann, C. Vergara, and R. Latorre. (1999) Acute activation of Maxi-K channels (hSlo) by estradiol binding to the beta subunit Science 285:1929-1931.

Weinstein R. S. et al., (2002) J Clin.Invest 109, 1041-1048.

Weinstein, R. S. (2000) J Bone Miner.Res 15, 621-625.

Whitmarsh, A. J., Shore, P., Sharrocks, A. D., Davis, R. J. (1995) Science 269, 403-407.

Wyckoff, M. H., K. L. Chambliss, C. Mineo, I. S. Yuhanna, M. E. Mendelsohn, S. M. Mumby, and P. W. Shaul. (2001) Plasma Membrane Estrogen Receptors Are Coupled to Endothelial Nitric-oxide Synthase through Galpha i. J. Biol. Chem. 276:27071-27076.

Yamakoshi, Y., Otani, Y., Fujii, S. and Endo, Y. (2000) Dependence of estrogenic activity on the shape of the 4-alkyl substituent in simple phenols. Biol. Pharm. Bull., 23, 259-261.

Yang, E., J. Zha, J. Jockel, L. H. Boise, C. B. Thompson, and S. J. Korsmeyer. (1995) Bad, a heterodimeric partner for Bcl-XL and Bcl-2, displaces Bax and promotes cell death. Cell 80:285-291.

Zhou, X. M., Liu, Y., Payne, G., Lutz, R. J., Chittenden, T. (2000) J. Biol. Chem. 275, 25046-25051

What is claimed is:

1. A method comprising administering an ANGELS compound to a subject by a dosage regimen that is effective to increase or maintain a bone property selected from the group consisting of bone mass, bone density and bone strength.

2. The method of claim 1 in which the ANGELS compound is non-phenolic.

3. The method of claim 2 in which the ANGELS compound is selected from the group consisting of estrenediol, androstenediol, estranediol, androstanediol, nor-estrenediol, homo-estrenediol, seco-estrenediol, nor-androstenediol, homo-androstenediol, seco-androstenediol, nor-estranediol, homo-estranediol, seco-estranediol, nor-androstanediol, homo-androstanediol, seco-androstanediol, and estratrienol.

4. The method of claim 3 in which the ANGELS compound is an estrenediol or an androstenediol.

5. The method of claim 4 in which the estrenediol is a 5(10)-estrenediol.

6. The method of claim 5 in which the 5(10)-estrenediol is selected from the group consisting of 5(10)-estrene-3α,17α-diol, 5(10)-estrene-3α,17β-diol, 5(10)-estrene-3β,17α-diol, and 5(10)-estrene-3β,17β-diol.

7. The method of claim 4 in which the ANGELS compound is a 5(6)-estrenediol or a 5(6)-androstenediol.

8. The method of claim 7 in which the ANGELS compound is selected from the group consisting of 5(6)-estrene-3α,17α-diol, 5(6)-estrene-3α,17β-diol, 5(6)-estrene-3β,17α-diol, 5(6)-estrene-3β,17β-diol, 5(6)-androstene-3α,17α-diol, 5(6)-androstene-3α,17β-diol, 5(6)-androstene-3β,17α-diol, and 5(6)-androstene-3α,17β-diol.

9. The method of claim 4 in which the ANGELS compound is a 4-estrenediol or a 4-androstenediol.

10. The method of claim 9 in which the ANGELS compound is selected from the group consisting of 4-estrene-3α,17α-diol, 4-estrene-3α,17β-diol, 4-estrene-3β,17α-diol, 4-estrene-3β,17α-diol, 4-androstene-3α,17α-diol, 4-androstene-3α,17β-diol, 4-androstene-3β,17α-diol, and 4-androstene-3α,17β-diol.

11. The method of claim 3 in which the ANGELS compound is an estranediol or an androstanediol.

12. The method of claim 11 in which the ANGELS compound is selected from the group consisting of estrane-3α,17α-diol, estrane-3α,17β-diol, estrane-3β,17α-diol, estrane-3β,17β-diol, androstane-3α,17α-diol, androstane-3α,17β-diol, androstane-3β,17α-diol, and androstane-3β,17β-diol

13. The method of claim 11 in which the ANGELS compound is a 5α-estranediol or a 5α-androstanediol.

14. The method of claim 12 in which the ANGELS compound is selected from the group consisting of 5α-estrane-3α,17α-diol, 5α-estrane-3α,17β-diol, 5α-estrane-3β,17α-diol, 5α-estrane-3β,17α-diol, 5α-androstane-3α,17α-diol, 5α-androstane-3α,17β-diol, 5α-androstane-3β,17α-diol, and 5α-androstane-3β,17β-diol.

15. The method of claim 11 in which the ANGELS compound is a 5β-estranediol or a 5β-androstanediol.

16. The method of claim 15 in which the ANGELS compound is selected from the group consisting of 5β-estrane-3α,17α-diol, 5β-estrane-3α,17β-diol, 5β-estrane-3β,17α-diol, 5β-estrane-3β,17β-diol, 5β-androstane-3α,17α-diol, 5β-androstane-3α,17β-diol, 5β-androstane-3β,17α-diol, and 5β-androstane-3β,17β-diol.

17. The method of claim 3 in which the ANGELS compound is selected from the group consisting of nor-estrenediol, homo-estrenediol, seco-estrenediol, nor-androstenediol, homo-androstenediol, seco-androstenediol, nor-estranediol, homo-estranediol, seco-estranediol, nor-androstanediol, homo-androstanediol, and seco-androstanediol.

18. The method of claim 17 in which the ANGELS compound is selected from the group consisting of nor-estrenediol, homo-estrenediol, and seco-estrenediol.

19. The method of claim 17 in which the ANGELS compound is selected from the group consisting of nor-estranediol, homo-estranediol, and seco-estranediol.

20. The method of claim 17 in which the ANGELS compound is selected from the group consisting of nor-androstenediol, homo-androstenediol, and seco-androstenediol.

21. The method of claim 17 in which the ANGELS compound is selected from the group consisting of nor-androstanediol, homo-androstanediol, and seco-androstanediol.

22. The method of claim 3 in which the ANGELS compound is an estratrienol.

23. The method of claim 20 in which the estratrienol is selected from the group consisting of estratrien-2-ol, estratrien-3-ol, estratrien-4-ol, and estratrien-5-ol.

24. The method of claim 20 in which the estratrienol is selected from the group consisting of seco-estratrienol, nor-estratrienol, and homo-estratrienol.

25. The method of claim 20 in which the estratrienol is selected from the group consisting of 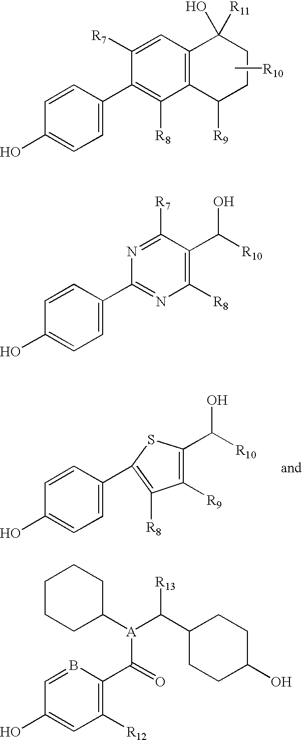
wherein R₇, R₈, R₉, R₁₀, R₁₁, and R₁₃ are each individually selected from the group consisting of hydrogen, C₁-C₅ alkyl and trifluoromethyl; A and B are each independently CH or N; and R₁₂ is selected from the group consisting of hydrogen, hydroxy, and C₁-C₅ alkyl.

26. The method of claim 25 in which R₇, R₈, R₉, R₁₀, R₁₁, and R₁₃ are each individually selected from the group consisting of hydrogen, methyl, ethyl, and trifluoromethyl.

27. The method of claim 1 in which the ANGELS compound is selected from the group consisting of 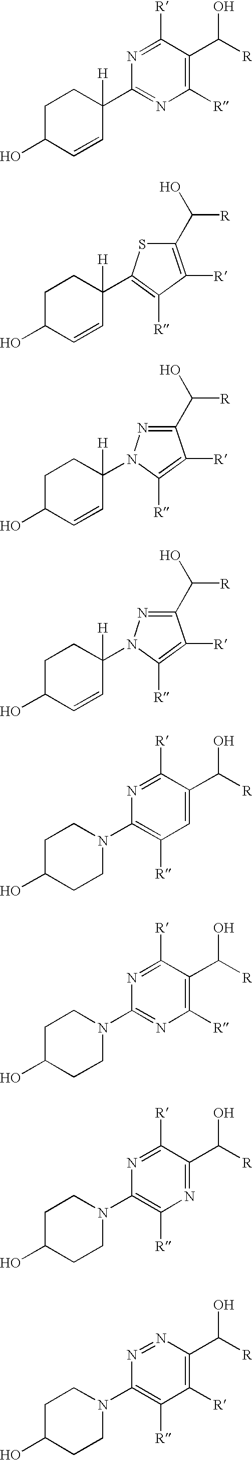
wherein R is hydrogen or C₁-C₅ alkyl; and wherein R′ and R″ are each individually selected from the group consisting of hydrogen, C₁-C₅ alkyl, trifluoromethyl, phenyl, and C₁-C₅ alkyl-substituted phenyl.

28. The method of claim 27 in which R is selected from the group consisting of hydrogen, methyl, and ethyl, and in which R′ and R″ are each individually selected from the group consisting of hydrogen, methyl, ethyl, propyl, trifluoromethyl, phenyl, 2-toluyl, 3-toluyl, and 4-toluyl.

29. The method of claim 1 in which the ANGELS compound is selected from the group consisting of 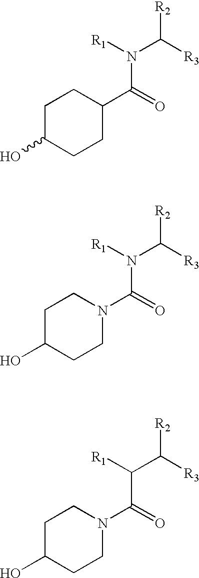
wherein R₁ is selected from the group consisting of hydrogen, C₁-C₅ alkyl, cycloalkyl, phenyl, and C₁-C₅ alkyl phenyl; R₂ is selected from the group consisting of hydrogen, C₁-C₅ alkyl, and trifluoromethyl; and R₃ is selected from the group consisting of hydrogen, C₁-C₅ alkyl, cycloalkyl, hydroxycycloalkyl, phenyl, and C₁-C₅ alkyl phenyl.

30. The method of claim 29 in which R₁ is selected from the group consisting of hydrogen, methyl, ethyl, isopropyl, cyclohexyl, and phenyl; R₂ is selected from the group consisting of hydrogen, methyl, ethyl, isopropyl, and trifluoromethyl; and R₃ is selected from the group consisting of hydrogen, methyl, ethyl, isopropyl, phenyl, cyclohexyl, cyclopentyl, and 4-hydroxycyclohexyl.

31. The method of claim 1 in which the subject suffers from a bone disorder.

32. The method of claim 31 in which the bone disorder is selected from the group consisting of osteoporosis, Paget's disease, osteogenesis imperfecta, chronic hyperparathyroidism, hyperthyroidism, rheumatoid arthritis, Gorham-Stout disease, McCune-Albright syndrome, osteometastases of cancer, osteometastases of multiple myeloma and alveolar ridge bone loss.

33. The method of claim 32 in which the bone disorder is osteoporosis.

34. The method of claim 33 in which the osteoporosis is selected from the group consisting of postmenopausal, male, senile, glucocorticoid-induced, alcohol-induced, anorexia/amenorhea-related, immobilization-induced, weightlessness-induced, post-transplantation, migratory, idiopathic, and juvenile.

35. The method of claim 1 in which the bone property is bone mass.

36. The method of claim 1 in which the bone property is bone density.

37. The method of claim 1 in which the bone property is bone strength.

38. A method comprising administering an ANGELS compound to a subject by a dosage regimen that is effective to provide a treatment selected from the group consisting of increase libido, control vasomotor disturbance, promote vasodilation, reduce bone loss, reduce mood swings, lower cholesterol, decrease low density lipoproteins (LDL), increase high density lipoproteins (HDL), slow atherosclerosis, slow progression of cancer, slow progression of cardiovascular disease, slow age-related neurodegeneration, slow progression of neurodegenerative disease, reduce risk of cancer, reduce risk of cardiovascular disease, reduce risk of stroke, and reduce risk of neurodegenerative disease.

39. The method of claim 38 in which the dosage regimen is effective to control a vasomotor disturbance or promote vasodilation.

40. The method of claim 38 in which the dosage regimen is effective to slow progression of cardiovascular disease, slow atherosclerosis, reduce risk of cardiovascular disease, or reduce risk of stroke.

41. The method of claim 38 in which the dosage regimen is effective to lower cholesterol, decrease LDL, or increase HDL.

42. The method of claim 38 in which the dosage regimen is effective to slow age-related neurodegeneration, slow progression of neurodegenerative disease, or reduce risk of neurodegenerative disease.

43. The method of claim 38 in which the dosage regimen is effective to increase libido.

44. The method of claim 38 in which the dosage regimen is effective to reduce bone loss.

45. The method of claim 38 in which the dosage regimen is effective to reduce mood swings.

46. The method of claim 38 in which the dosage regimen is effective to reduce risk of cancer or slow progression of cancer.

47. A pharmaceutical composition comprising a compound represented by a formula selected from the group consisting of 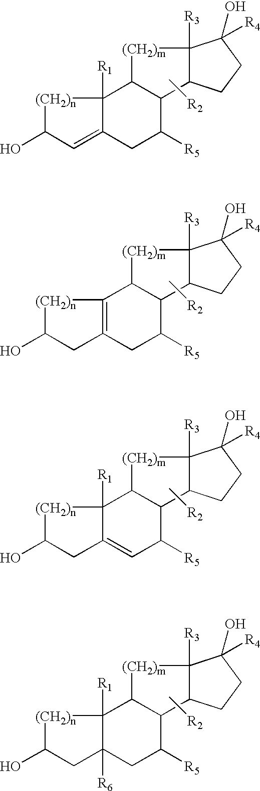
wherein R₁, R₃ and R₆ are each individually hydrogen or methyl;
wherein m and n are each individually integers in the range of 1 to 3; and
wherein R₂ and R₅ are each individually selected from the group consisting of hydrogen, halogen, mercapto, hydroxyl, cyano, amino, ethenyl, ethynyl, aryl, C₁-C₅ heteroaryl, C₁-C₅ alkyl, C₁-C₅ cycloalkyl, C₁-C₅ haloalkyl, C₁-C₅ alkylthio, C₁-C₅ ester, C₁-C₅ alkoxy, C₁-C₅ acyl, C₁-C₅ alkylamine, and C₁-C₅ acyloxy; and
wherein R₄ is selected from the group consisting of hydrogen, ethenyl, ethynyl, aryl, C₁-C₅ heteroaryl, C₁-C₅ alkyl, C₁-C₅ cycloalkyl, C₁-C₅ haloalkyl, C₁-C₅ ester, and C₁-C₅ acyl.

48. The pharmaceutical composition of claim 47 in which the compound is represented by the formula

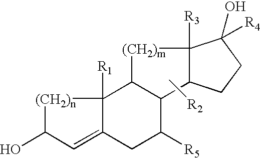

49. The pharmaceutical composition of claim 48 in which n is 1 or 3.

50. The pharmaceutical composition of claim 48 in which m is 1 or 3.

51. The pharmaceutical composition of claim 48 in which the compound is represented by the formula

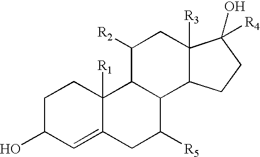

52. The pharmaceutical composition of claim 51 in which R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

53. The pharmaceutical composition of claim 47 in which the compound is represented by the formula

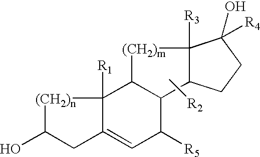

54. The pharmaceutical composition of claim 53 in which n is 1 or 3.

55. The pharmaceutical composition of claim 53 in which m is 1 or 3.

56. The pharmaceutical composition of claim 53 in which the compound is represented by the formula

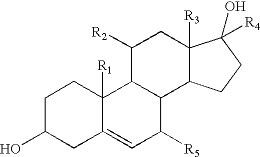

57. The pharmaceutical composition of claim 56 in which R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

58. The pharmaceutical composition of claim 47 in which the compound is represented by the formula

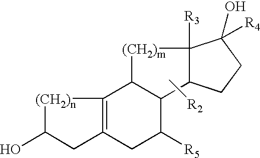

59. The pharmaceutical composition of claim 58 in which n is 1 or 3.

60. The pharmaceutical composition of claim 58 in which m is 1 or 3.

61. The pharmaceutical composition of claim 58 in which the compound is represented by the formula

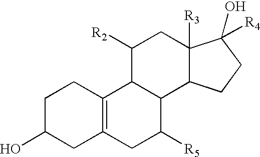

62. The pharmaceutical composition of claim 61 in which in which R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

63. The pharmaceutical composition of claim 47 in which the compound is represented by the formula

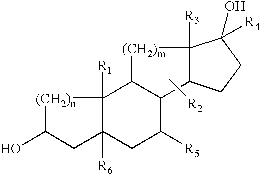

64. The pharmaceutical composition of claim 63 in which n is 1 or 3.

65. The pharmaceutical composition of claim 63 in which m is 1 or 3.

66. The pharmaceutical composition of claim 63 in which the compound is represented by the formula

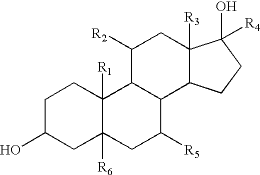

67. The pharmaceutical composition of claim 66 in which in which R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

68. A pharmaceutical composition comprising a compound represented by a formula selected from the group consisting of 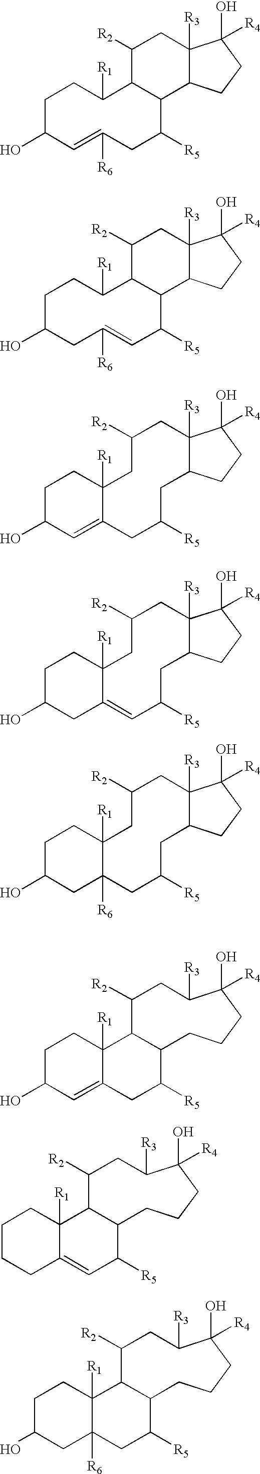
wherein R₁, R₃ and R₆ are each individually hydrogen or methyl;
wherein R₂ and R₅ are each individually selected from the group consisting of hydrogen, halogen, mercapto, hydroxyl, cyano, amino, ethenyl, ethynyl, aryl, C₁-C₅ heteroaryl, C₁-C₅ alkyl, C₁-C₅ cycloalkyl, C₁-C₅ haloalkyl, C₁-C₅ alkylthio, C₁-C₅ ester, C₁-C₅ alkoxy, C₁-C₅ acyl, C₁-C₅ alkylamine, and C₁-C₅ acyloxy; and
wherein R₄ is selected from the group consisting of hydrogen, ethenyl, ethynyl, aryl, C₁-C₅ heteroaryl, C₁-C5 alkyl, C₁-C₅ cycloalkyl, C₁-C₅ haloalkyl, C₁-C₅ ester, and C₁-C₅ acyl.

69. The pharmaceutical composition of claim 68 in which the compound is represented by a formula selected from the group consisting of

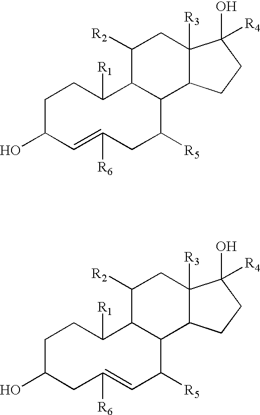

70. The pharmaceutical composition of claim 69 in which R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

71. The pharmaceutical composition of claim 68 in which the compound is represented by a formula selected from the group consisting of

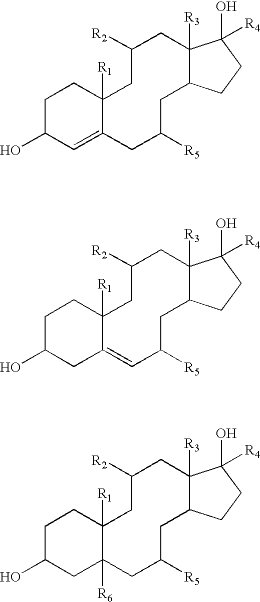

72. The pharmaceutical composition of claim 71 in which R₂ is selected from the group consisting of hydrogen, C₁-C₅ alkyl, phenyl, and C₁-C₅ alkyl substituted phenyl; R₄ is selected from the group consisting of hydrogen, C₁-C₅ alkyl and ethynyl; and R₅ is selected from the group consisting of hydrogen and C₁-C₅ alkyl.

73. The pharmaceutical composition of claim 68 in which the compound is represented by a formula selected from the group consisting of

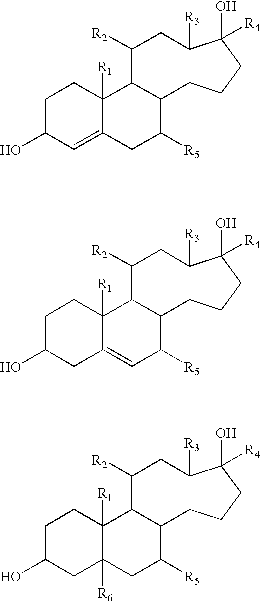

74. The pharmaceutical composition of claim 73 in which R₂ is selected from the group consisting of hydrogen, C₁-C₅ alkyl, phenyl, and C₁-C₅ alkyl-substituted phenyl; R₄ is selected from the group consisting of hydrogen, C₁-C₅ alkyl and ethynyl; and R₅ is selected from the group consisting of hydrogen and C₁-C₅ alkyl.

75. A pharmaceutical composition comprising a compound represented by a formula selected from the group consisting of 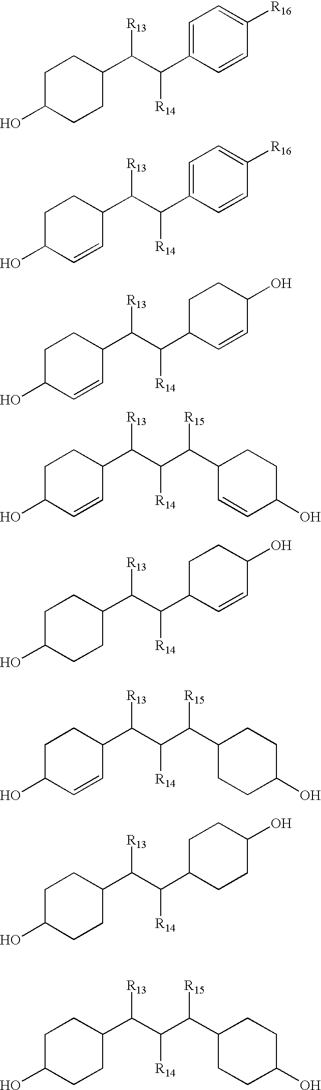
wherein R₁₃, R₁₄, and R₁₅ are each individually selected from the group consisting of hydrogen, ethenyl, ethynyl, C₁-C₅ alkyl, cycloalkyl and phenyl; and wherein R₁₆ is selected from the group consisting of hydrogen, hydroxyl, and C₁-C₅ hydroxyalkyl.

76. The pharmaceutical composition of claim 75 in which the compound is represented by a formula selected from the group consisting of

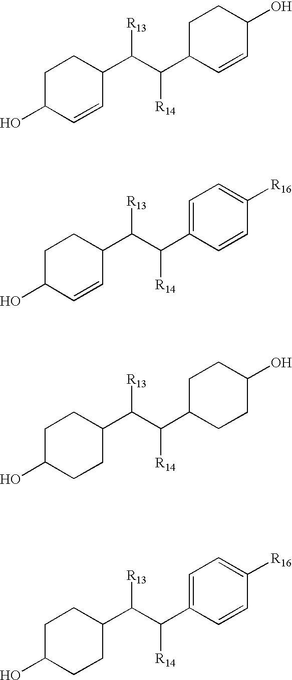

77. The pharmaceutical composition of claim 76 in which R₁₃ and R₁₄ are each individually selected from the group consisting of hydrogen, C₁-C₅ alkyl, cycloalkyl and phenyl; and in which R₁₆ is hydroxyl.

78. The pharmaceutical composition of claim 75 in which the compound is represented by a formula selected from the group consisting of

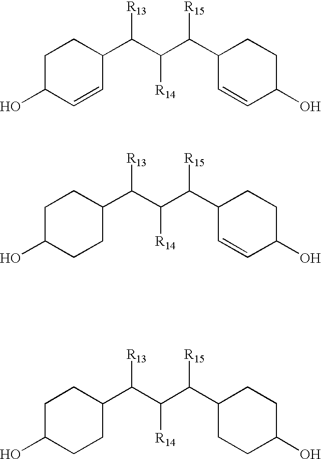

79. The pharmaceutical composition of claim 78 in which R₁₃, R₁₄ and R₁₅ are each individually selected from the group consisting of hydrogen, C₁-C₅ alkyl, cycloalkyl and phenyl.

80. The pharmaceutical composition of claim 75 in which the compound is represented by a formula selected from the group consisting of

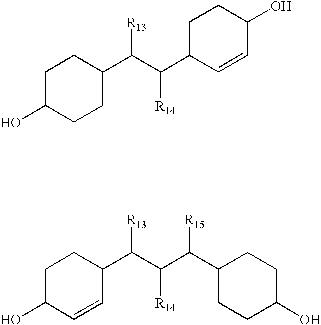

81. The pharmaceutical composition of claim 80 in which R₁₃, R₁₄ and R₁₅ are each individually selected from the group consisting of hydrogen, C₁-C₅ alkyl, cycloalkyl and phenyl.

82. A pharmaceutical composition comprising a compound represented by a formula selected from the group consisting of 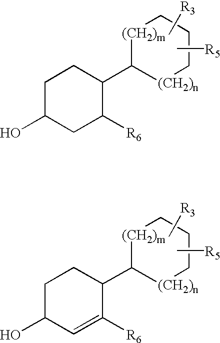
in which m and n are each individually integers in the range of 1 to 4; R₃ and R₅ are each individually selected from the group consisting of hydroxy, hydrogen, C₁ to C₅ alkyl, C₁ to C₅ hydroxyalkyl, C₁ to C₅ alkoxy, C₁ to C₅ thioalkoxy, phenyl, and C₁ to C₅ alkyl-substituted phenyl; and in which R₆ is selected from the group consisting of hydrogen and C₁-C₅ alkyl.

83. The pharmaceutical composition of claim 82 in which the compound is represented by a formula selected from the group consisting of

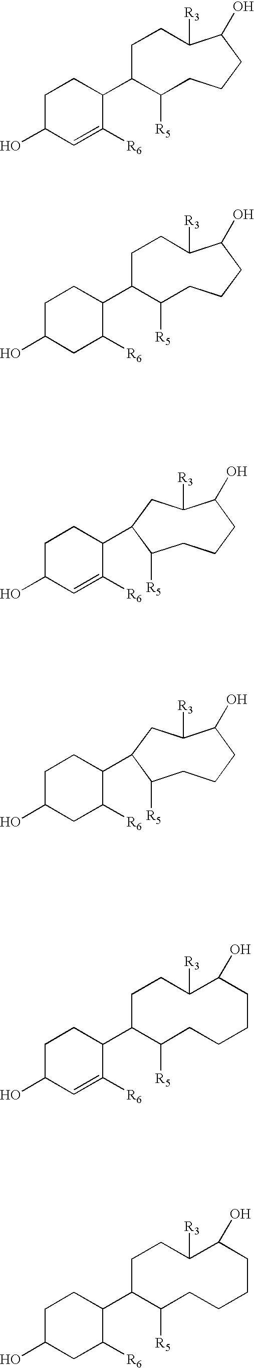

84. The pharmaceutical composition of claim 83 in which R₃ is selected from the group consisting of hydrogen, methyl and ethyl; and in which R₅ and R₆ are each individually selected from the group consisting of hydrogen and C₁-C₅ alkyl.
